(19) United States
(12) Patent Application Publication
(10) Pub. No.: US 2026/0337676 A1
(43) Pub. Date: Oct. 29, 2026

(54) COMPOSITIONS AND METHODS FOR SEQUENCING MULTIPLE REGIONS OF A TEMPLATE MOLECULE USING READ-CAPPING NUCLEOTIDE ANALOGS

(71) Applicant: Element Biosciences, Inc., San Diego, CA (US)

(72) Inventors: Junhua ZHAO, San Diego, CA (US); Su ZHANG, Rosemead, CA (US); Samantha SNOW, San Diego, CA (US); James GHADIALI, San Diego, CA (US); Matthew KELLINGER, San Diego, CA (US); Ryan KELLEY, San Diego, CA (US); Tyler LOPEZ, Valley Center, CA (US); Mariam DAWOOD, El Cajon, CA (US); Tsung-Li LIU, San Diego, CA (US); Daniel HONIGFORT, San Diego, CA (US); Michael KIM, El Cajon, CA (US); Marco TJIOE, San Diego, CA (US); Keerthana ELANGO, San Diego, CA (US)

(21) Appl. No.: 19/698,987

(22) Filed: Jun. 5, 2026

Related U.S. Application Data
(63) Continuation of application No. PCT/IB2024/062286, filed on Dec. 5, 2024.
(60) Provisional application No. 63/607,234, filed on Dec. 7, 2023.

(51) Int. Cl. C12Q 1/6869; C12Q 1/48; C12Q 1/6834; C12Q 1/6876

(52) U.S. Cl. CPC C12Q 1/6869; C12Q 1/48; C12Q 1/6834; C12Q 1/6876; C12Y 207/07

(57) ABSTRACT

The present disclosure provides compositions comprising read-capping nucleotide analogs, and methods using the read-capping nucleotide analogs for sequencing. The read-capping nucleotide analogs can be incorporated at the terminal 3′ end of a sequencing read product when sequencing a first region of a template molecule is completed to generate a first capped sequencing read product. The first capped sequencing read products are not removed from the template molecules prior to sequencing a second region of the template molecules. Removal of the first capped sequencing read product from the template molecule is obviated because the incorporated read-capping nucleotide analog blocks binding and incorporation of nucleotide reagents used to sequence a second region of the nucleic acid template molecule. Using the read-capping nucleotide analogs, without the need to remove the sequencing read products, reduces damage to the template molecules and preserves intact template molecules after numerous sequencing cycles.

### CROSS-REFERENCE TO PRIORITY APPLICATIONS

This application is a continuation of International Application No. PCT/IB 2024/062286, filed Dec. 5, 2024, which claims the benefit of and priority to U.S. Provisional Patent Application No. 63/607,234, filed Dec. 7, 2023, the entire contents of each of which are incorporated herein by reference.

### TECHNICAL FIELD

The present disclosure relates to the field of high throughput sequencing, and provides compositions comprising read-capping nucleotide analogs, and methods using the read-capping nucleotide analogs.

### BACKGROUND

Traditional multiplex sequencing workflows require sequencing a first region (e.g., a sequence-of-interest region) using a first sequencing primer, and sequencing a second region (e.g., a sample index region) using a second sequencing primer. The sequencing read products that are synthesized from sequencing the first region must be efficiently removed prior to sequencing the second region, while leaving the template molecule undamaged. Traditional sequencing workflows that employ chemical-based reagents (e.g., NaOH and/or formamide) to denature the sequencing read products can be harsh, resulting in damage to the template molecule. Gentler chemical-based denaturation conditions may be less damaging to the template molecule, but do not effectively remove the sequencing read products which contribute to residual signals. Accumulation of residual signals in subsequent sequencing cycles can reduce sequencing accuracy. There thus exists a need for additional methods to efficiently remove sequencing read products from previous sequencing cycles, or prevent previous sequencing read products from affecting subsequent sequencing cycles. The present disclosure provides compositions and methods for sequentially sequencing different regions of the same nucleic acid template molecule and reducing residual signals that contribute to poor sequencing quality.

### SUMMARY

The disclosure provides methods for sequencing two or more regions of a nucleic acid template molecule, comprising: (a) providing a plurality of nucleic acid template molecules, wherein individual template molecules comprise (i) a first region and a first universal sequencing primer binding site and (ii) a second region and a second universal sequencing primer binding site; (b) providing a plurality of first nucleic acid sequencing primers and a plurality of second nucleic acid sequencing primers; (c) hybridizing individual first nucleic acid sequencing primers to the first universal sequencing primer binding sites on the template molecules; (d) sequencing the first regions of the plurality of nucleic acid template molecules, thereby generating a plurality of first sequencing read products; (e) conducting a plurality of first capping reactions comprising incorporating a read-capping nucleotide analog into the terminal 3′ ends of individual first sequencing read products, thereby generating a plurality of capped first sequencing read products, wherein the read-capping nucleotide analog comprises (i) a heterocyclic base, (ii) a sugar, and (iii) a polyphosphate chain, wherein the heterocyclic base is linked to a terminating moiety that blocks binding of a multivalent molecule to the terminal 3′ ends of the capped first sequencing read products, wherein individual multivalent molecules comprise a core attached to multiple nucleotide arms and individual nucleotide arms are attached to a nucleotide unit; and wherein the terminating moiety blocks polymerase-catalyzed incorporation of a subsequent nucleotide into the capped first sequencing read products, (f) retaining the plurality of capped first sequencing read products which are hybridized to nucleic acid template molecules, wherein the terminating moiety is not cleaved or removed; (g) hybridizing individual second nucleic acid sequencing primers to the second universal sequencing primer binding sites on the individual template molecules; and (h) sequencing the second regions on the plurality of template molecules, thereby generating a plurality of second sequencing read products.

In some embodiments of the methods of the disclosure, the methods further comprise: (i) conducting a plurality of second capping reactions comprising incorporating a read-capping nucleotide analog into the terminal 3′ ends of individual second sequencing read products, thereby generating a plurality of capped second sequencing read products, wherein the read-capping nucleotide analog comprises (i) a heterocyclic base, (ii) a sugar, and (iii) a polyphosphate chain, wherein the heterocyclic base is linked to a terminating moiety that blocks binding of a multivalent molecule to the terminal 3′ ends of the capped second sequencing read products, wherein individual multivalent molecules comprise a core attached to multiple nucleotide arms and individual nucleotide arms are attached to a nucleotide unit; wherein the terminating moiety blocks polymerase-catalyzed incorporation of a subsequent nucleotide into the capped second sequencing read products; and (j) retaining the plurality of capped second sequencing read products which are hybridized to nucleic acid template molecules, wherein the terminating moiety is not cleaved or removed.

In some embodiments of the methods of the disclosure, individual sequenced template molecules comprise at least one capped first sequencing read product hybridized thereon, and at least one capped second sequencing read product hybridized thereon.

In some embodiments of the methods of the disclosure, the read-capping nucleotide analog that is incorporated at the terminal 3′ end of individual capped first sequencing read products is not modified to convert the sugar 3′ position into an extendible 3′OH group. In some embodiments, the read-capping nucleotide analog that is incorporated at the terminal 3′ end of individual capped second sequencing read products is not modified to convert the sugar 3′ position into an extendible 3′OH group.

In some embodiments of the methods of the disclosure, the heterocyclic base is linked to the terminating moiety by an alkyne linkage, alkene linkage, or alkane linkage.

In some embodiments of the methods of the disclosure, the terminating moiety comprises a polymer moiety. In some embodiments, the polymer moiety is selected from the group consisting of a polyether moiety, a polyethylene glycol moiety, polypropylene glycol moiety, polyvinyl acetate moiety, polylactic acid moiety, polyglycolic acid moiety, a polyamide and a polyester moiety. In some embodiments, the polymer moiety comprises a poly(glycerol) moiety, a poly(oxazoline) moiety, a poly(hydroxypropyl methacrylate) moiety (PHPMA), a poly(2-hydroxyethyl methacrylate) moiety (PHEMA), a poly(N-(2-hydroxypropyl) methacrylamide) moiety (HPMA), a poly(vinylpyrrolidone) moiety (PVP), a poly(N,N-dimethyl acrylamide) moiety (PDMA), or a poly(N-acryloylmorpholine) moiety (PACM).

In some embodiments of the methods of the disclosure, the terminating moiety comprises a polyethylene glycol (PEG) moiety. In some embodiments, the PEG moiety has a molecular weight of 1K-20K (KiloDaltons, or KDa). In some embodiments, the terminating moiety comprises a propargylamino moiety, an allylamino moiety, a propylamino moiety, an ethylmercapto moiety, a hydroxymethyl moiety, an arylmercapto moiety, a 1-X-1H-1,2,3-triazol-4-yl moiety, a 5-X-1H-1,2,3-triazol-1-methyl moiety, a hydrazone moiety or an O-alkyl oxime moiety, wherein “X” comprises a polymer.

In some embodiments of the methods of the disclosure, the heterocyclic base comprises a chain terminating moiety at the 3′ sugar group. In some embodiments, the chain terminating moiety comprises an alkyl group, alkenyl group, alkynyl group, allyl group, aryl group, benzyl group, azide group, azido group, O-azidomethyl group, amine group, amide group, keto group, isocyanate group, phosphate group, thio group, disulfide group, carbonate group, urea group, or silyl group.

In some embodiments of the methods of the disclosure, individual nucleic acid template molecules in the plurality of nucleic acid template molecules are single-stranded or double-stranded nucleic acid molecules.

In some embodiments of the methods of the disclosure, the plurality of nucleic acid template molecules are immobilized to a support or immobilized to a coating on a support. In some embodiments, individual nucleic acid template molecules in the plurality of nucleic acid template molecules are covalently joined to an immobilized surface capture primer, or wherein individual template molecules in the plurality are hybridized to an immobilized surface capture primer, wherein the immobilized surface capture primer is immobilized on the support.

In some embodiments of the methods of the disclosure, at least one of the nucleic acid template molecules in the plurality of nucleic acid template molecules comprises a uridine, or wherein at least one of the nucleic acid template molecules in plurality of nucleic acid template molecules lacks a uridine.

In some embodiments of the methods of the disclosure, individual nucleic acid template molecules in the plurality of plurality of nucleic acid template molecules comprise clonally amplified nucleic acid molecules. In some embodiments, individual nucleic acid template molecules in the plurality of nucleic acid template molecules comprise at least one copy of a sequence-of-interest and at least one universal adaptor sequence. In some embodiments, individual nucleic acid template molecules in the plurality of nucleic acid template molecules comprise concatemer template molecules having two or more tandem repeat units, wherein individual repeat units comprise a sequence-of-interest and at least one universal adaptor sequence.

In some embodiments of the methods of the disclosure, the support comprises glass or plastic. In some embodiments, the support is configured on a flowcell, or an interior of a capillary lumen. In some embodiments, the support comprises at least one hydrophilic polymer coating layer and a plurality of surface capture primers immobilized to the at least one hydrophilic polymer coating layer, and wherein the at least one hydrophilic polymer coating layer has a water contact angle of no more than 45 degrees. In some embodiments, the at least one hydrophilic polymer coating layer comprises polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, or dextran. In some embodiments, the at least one hydrophilic polymer coating layer comprises polymer molecules having a molecular weight of at least 1000 Daltons. In some embodiments, the at least one hydrophilic polymer coating layer comprises branched polymer molecules having 4-8 branches. In some embodiments, the support comprises: a) a first coating layer comprising a first monolayer of hydrophilic polymer molecules tethered to the support; b) a second coating layer comprising a second monolayer of hydrophilic polymer molecules tethered to the first monolayer; and c) a third coating layer comprising a third monolayer of hydrophilic polymer molecules tethered to the second monolayer, and wherein the hydrophilic polymer molecules of the first layer, second layer or third layer comprise branched polymer layers.

In some embodiments of the methods of the disclosure, the plurality of surface capture primers are immobilized to the hydrophilic polymer molecules of the second monolayer or third monolayer, and the surface capture primers are distributed at a plurality of depths throughout the second layer or the third layer. In some embodiments, one or more of the at least one hydrophilic polymer coating layers comprise a plurality of surface capture primers at a surface density of least 1000/μm².

In some embodiments of the methods of the disclosure, the methods comprise contacting the plurality of nucleic acid template molecules with (i) the first plurality of sequencing primers, wherein the individual nucleic acid template molecules are immobilized and the plurality of sequencing primers are soluble, (ii) a plurality of sequencing polymerases, and (iii) a plurality of nucleotide reagents, under conditions suitable for hybridizing soluble sequencing primers to individual nucleic acid template molecules to generate a plurality of nucleic acid duplexes on the individual nucleic acid template molecules, and wherein the conditions are suitable for binding nucleic acid duplexes with sequencing polymerases and nucleotide reagents.

In some embodiments of the methods of the disclosure, individual nucleotide reagents in the plurality of nucleotide reagents comprise an aromatic base, a five carbon sugar and 1-10 phosphate groups. In some embodiments, the plurality of nucleotide reagents comprises one or more types of nucleotide reagent selected from the group consisting of dATP, dGTP, dCTP, dTTP and dUTP. In some embodiments, the plurality of nucleotide reagents comprises a combination of two or more types of nucleotide reagent selected from the group consisting of dATP, dGTP, dCTP, dTTP and dUTP. In some embodiments, at least one nucleotide reagent in the plurality of nucleotide reagents lacks a detectable reporter moiety. In some embodiments, at least one nucleotide reagent in the plurality of nucleotide reagents is labeled with a detectable reporter moiety.

In some embodiments of the methods of the disclosure, individual nucleotide reagents in the plurality of nucleotide reagents comprise at least one chain terminating nucleotide comprising (i) an aromatic base, (ii) a sugar having a 3′ chain terminating moiety that inhibits polymerase-catalyzed nucleotide incorporation, and (iii) 1-10 phosphate groups. In some embodiments, the at least one chain terminating nucleotide comprises a removable chain terminating moiety at the 3′ sugar group. In some embodiments, the removable chain terminating moiety comprises an alkyl group, alkenyl group, alkynyl group, allyl group, aryl group, benzyl group, azide group, azido group, O-azidomethyl group, amine group, amide group, keto group, isocyanate group, phosphate group, thio group, disulfide group, carbonate group, urea group, or silyl group. In some embodiments, the at the at least one chain terminating nucleotide comprises a removable chain terminating moiety at the 3′ sugar group. In some embodiments, the removable chain terminating moiety comprises a 3′-O-amino group, a 3′-O-aminomethyl group, a 3′-O-methylamino group, or derivatives thereof. In some embodiments, the at least one chain terminating moiety is cleavable or removable with a chemical compound to generate an extendible 3′ OH moiety on the sugar group.

In some embodiments of the methods of the disclosure, sequencing the first region on the plurality of template molecules comprises: (a) contacting the plurality of nucleic acid template molecules with (i) a first plurality of sequencing polymerases and (ii) the plurality of first nucleic acid sequencing primers, wherein the nucleic acid template molecules are immobilized and the plurality of first nucleic acid sequencing primers are soluble, and wherein the contacting is conducted under conditions suitable to form a plurality of complexed polymerases comprising a sequencing polymerase bound to a nucleic acid duplex, wherein the nucleic acid duplex comprises a portion of an individual nucleic acid template molecule hybridized to an individual first nucleic acid sequencing primer; (b) contacting the plurality of complexed sequencing polymerases with a plurality of nucleotides under conditions suitable for binding at least one nucleotide to a complexed sequencing polymerase, wherein the plurality of nucleotides comprises at least one nucleotide analog labeled with a fluorophore and having a removable chain terminating moiety at the sugar 3′ position; (c) incorporating at least one nucleotide into the 3′ ends of the first nucleic acid sequencing primers, thereby generating a plurality of first sequencing read products by extending the first nucleic acid sequencing primers; and (d) detecting the at least one nucleotide and identifying the nucleobase of the at least one nucleotide.

In some embodiments of the methods of the disclosure, the plurality of nucleotide reagents comprises at least one multivalent molecule. In some embodiments, the at least one multivalent molecule comprises: (1) a core; and (2) a plurality of nucleotide arms comprising (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, and wherein the linker is attached to the nucleotide unit. In some embodiments, the nucleotide unit comprises a base, sugar and 1-10 phosphate groups, and the linker is attached to the nucleotide unit through the base. In some embodiments, individual nucleotide arms of the at least one multivalent molecule comprise a linker having an aliphatic chain or an oligo ethylene glycol chain, and wherein the linker chain comprises 2-6 subunits. In some embodiments, the core of the at least one multivalent molecule comprises streptavidin and the core attachment moiety comprises biotin. In some embodiments, the plurality of nucleotide arms comprise the same type of a nucleotide unit, and wherein the type of nucleotide unit is selected from the group consisting of dATP, dGTP, dCTP, dTTP and dUTP.

In some embodiments of the methods of the disclosure, the plurality of nucleotide reagents comprises a plurality of multivalent molecules, wherein individual multivalent molecules in the plurality of multivalent molecules comprise the same type of nucleotide unit. In some embodiments, the type of nucleotide unit is selected from the group consisting of dATP, dGTP, dCTP, dTTP and dUTP. In some embodiments, the plurality of nucleotide reagents comprises a plurality of multivalent molecules comprising a mixture of two or more types of multivalent molecules, each type of multivalent molecules comprising one or more nucleotide unit types selected from the group consisting of dATP, dGTP, dCTP, dTTP and dUTP.

In some embodiments of the methods of the disclosure, the plurality of nucleotide reagents comprises at least one fluorophore-labeled multivalent molecule.

In some embodiments of the methods of the disclosure, sequencing the first regions comprises conducting a two-step sequencing method. In some embodiments, a first step comprises: (a) contacting the plurality of nucleic acid template molecules with (i) a first plurality of sequencing polymerases and (ii) the plurality of first sequencing primers, wherein the nucleic acid template molecules are immobilized and the plurality of first sequencing primers are soluble, and wherein the contacting is conducted under conditions suitable to form a plurality of first complexed polymerases, individual complexed polymerases comprising a sequencing polymerase bound to a nucleic acid duplex, wherein the nucleic acid duplex comprises a portion of the nucleic acid template molecule hybridized to a first soluble sequencing primer; (b) contacting the plurality of complexed sequencing polymerases with a plurality of nucleotide reagents comprising at least one multivalent molecule, wherein the at least one multivalent molecule is a detachably labeled multivalent molecule, wherein complementary nucleotide units of the multivalent molecules bind to at least two of the plurality of first complexed polymerases, thereby forming a plurality of multivalent-complexed polymerases, and wherein incorporation of the complementary nucleotide units into the first sequencing primers is inhibited; (c) detecting the plurality of multivalent-complexed polymerases; and (d) identifying the nucleobase of the complementary nucleotide units that are bound to the plurality of first complexed polymerases in the plurality of multivalent-complexed polymerases, thereby determining the sequence of the nucleic acid template. In some embodiments, the complementary nucleotide units are complementary to a nucleotide of the nucleic acid template molecule that is immediately 5′ of the nucleic acid duplex.

In some embodiments of the methods of the disclosure, sequencing the first region on the plurality of template molecules comprises forming an avidity complex, wherein the method comprises: (i) binding a first sequencing primer, a first sequencing polymerase, and a first detectably labeled multivalent molecule to a first region of a concatemer template molecule, thereby forming a first binding complex, wherein a first nucleotide unit of the first detectably labeled multivalent molecule binds to the first sequencing polymerase; (ii) binding a second sequencing primer, a second sequencing polymerase, and the first detectably labeled multivalent molecule to a second region of the concatemer template molecule, thereby forming a second binding complex, wherein a second nucleotide unit of the first multivalent molecule binds to the second sequencing polymerase, wherein the first and second binding complexes form an avidity complex, wherein the concatemer template molecule comprises two or more tandem repeat units, wherein each tandem repeat unit comprises a sequence of interest and a universal primer binding site that binds a first and a second universal sequencing primer, and wherein the contacting is conducted under conditions suitable to inhibit polymerase-catalyzed incorporation of the first and second nucleotide units into the first and second binding complexes, respectively; (iii) detecting the first and second binding complexes on the same concatemer template molecule, and (iv) identifying the first nucleotide unit in the first binding complex thereby determining the sequence of the first region of the concatemer template molecule, and identifying the second nucleotide unit in the second binding complex thereby determining the sequence of the second region of the concatemer template molecule.

In some embodiments of the methods of the disclosure, the second step comprises: (e) dissociating the plurality of multivalent-complexed polymerases and removing the plurality of first sequencing polymerases and multivalent molecules, and retaining the nucleic acid duplexes; (f) contacting the nucleic acid duplexes of step (e) with a plurality of second sequencing polymerases, wherein the contacting is conducted under conditions suitable for binding the plurality of second sequencing polymerases to the nucleic acid duplexes, thereby forming a plurality of second complexed polymerases; (g) contacting the plurality of second complexed polymerases with a plurality of nucleotides comprising at least one nucleotide analog having a removable chain terminating moiety, wherein the contacting is conducted under conditions suitable for binding complementary nucleotides present in the plurality of nucleotides to at least two complexed polymerases of the plurality of second complexed polymerases of step (f), thereby forming a plurality of nucleotide-complexed polymerases, and wherein the conditions are suitable for promoting incorporation of the bound complementary nucleotides into the first sequencing primers of the nucleotide-complexed polymerase, thereby generating the plurality of first sequencing read products. In some embodiments, the removable chain terminating moiety is at the sugar 3′ position. In some embodiments, the complementary nucleotides are complementary to a nucleotide of the nucleic acid template molecule that is immediately 5′ of the nucleic acid duplex.

### BRIEF DESCRIPTION OF THE DRAWINGS

FIG. 1 is a schematic showing an exemplary single stranded template molecule comprising: a second surface primer binding site (e.g., SP2; surface pinning primer binding site); a second index sequence (2^{nd} index); a first sequencing primer binding site (e.g., forward sequencing primer binding site, or FWD Seq); a sequence-of-interest (e.g., insert); a second sequencing primer binding site (e.g., reverse sequencing primer binding site, or REV Seq); a first index sequence (1^{st} index); and a first surface primer binding site (e.g., SP1; a surface capture primer binding site). The template molecule shown in FIG. 1 can comprise one copy template molecule having one copy of the sequence-of-interest and one copy of various universal adaptor sequences. Alternatively, the template molecule shown in FIG. 1 is one unit of a concatemer having two or more tandem copies of the unit depicted, and each unit comprises a sequence-of-interest and one copy of various universal adaptor sequences. In some embodiments, the first index sequence comprises a first sample index sequence. In some embodiments, the first sample index sequence comprises a short random sequence (e.g., NNN).

FIG. 2 is a schematic showing an exemplary single stranded template molecule comprising: a second surface primer binding site (e.g., SP2; surface pinning primer binding site); a first sequencing primer binding site (e.g., forward sequencing primer binding site, or FWD Seq); a first index sequence; a sequence-of-interest (e.g., insert); a second index sequence; a second sequencing primer binding site (e.g., reverse sequencing primer binding site, or REV Seq); and a first surface primer binding site (e.g., SP1; a capture primer binding site). The template molecule shown in FIG. 2 can comprise one copy template molecule having one copy of the sequence-of-interest and one copy of various universal adaptor sequences. Alternatively, the template molecule shown in FIG. 2 is one unit of a concatemer having two or more tandem copies of the unit depicted, and each unit comprises a sequence-of-interest and one copy of various universal adaptor sequences. In some embodiments, the first index sequence comprises a first sample index sequence. In some embodiments, the first sample index sequence comprises a short random sequence (e.g., NNN).

FIG. 3A is a schematic showing an immobilized concatemer template molecule comprising at least two tandem copies of a unit where each unit comprises an insert sequence (e.g., sequence-of-interest) and various universal sequences which comprise universal primer binding sites. The concatemer template molecule can lack a scissile moiety (e.g., uracil).

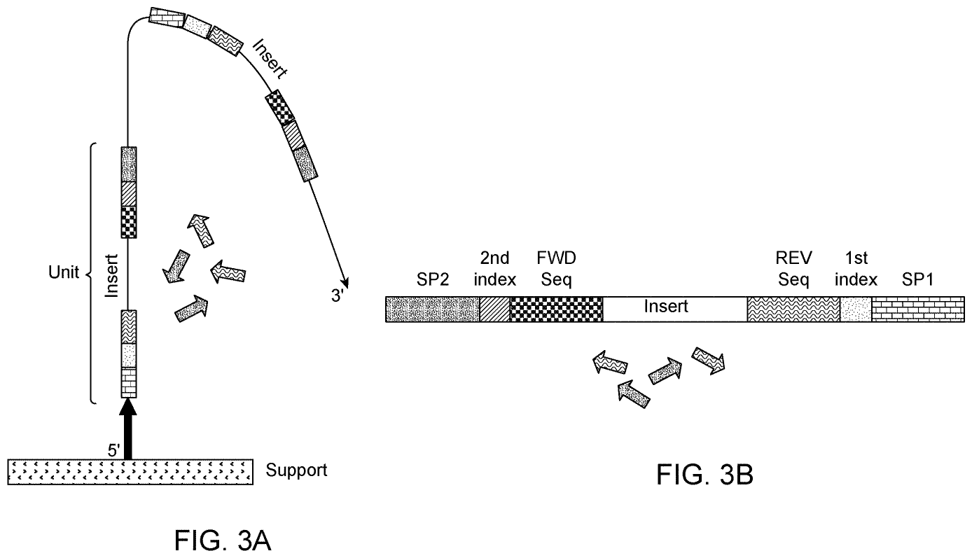

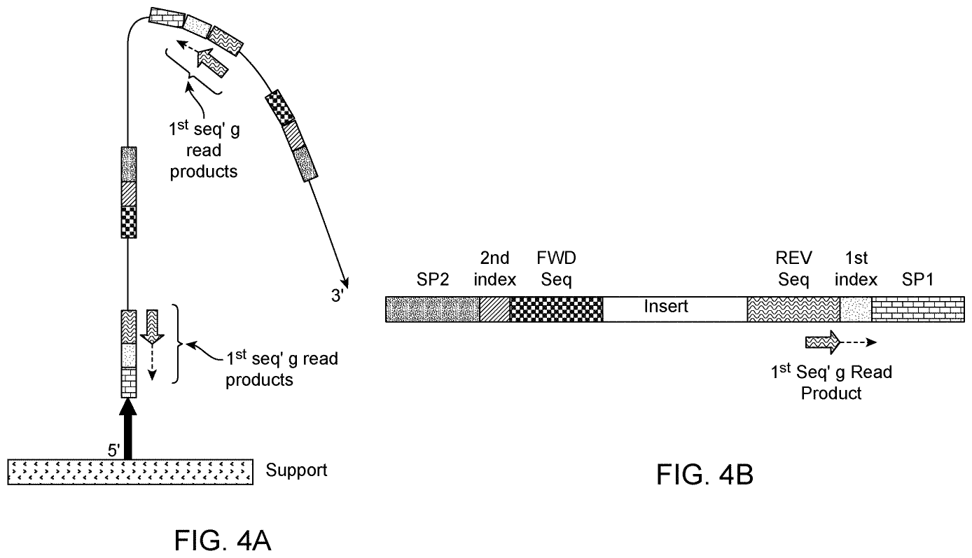

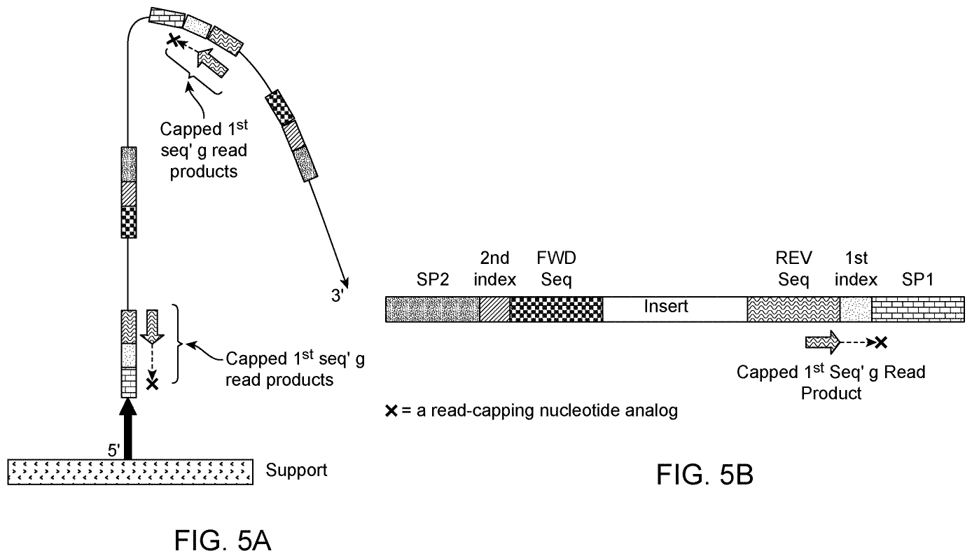

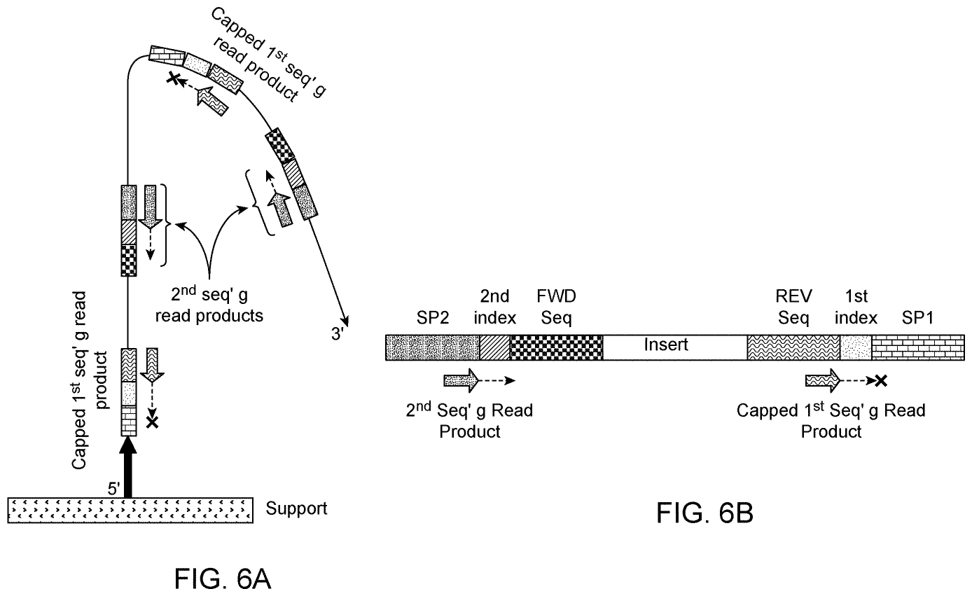

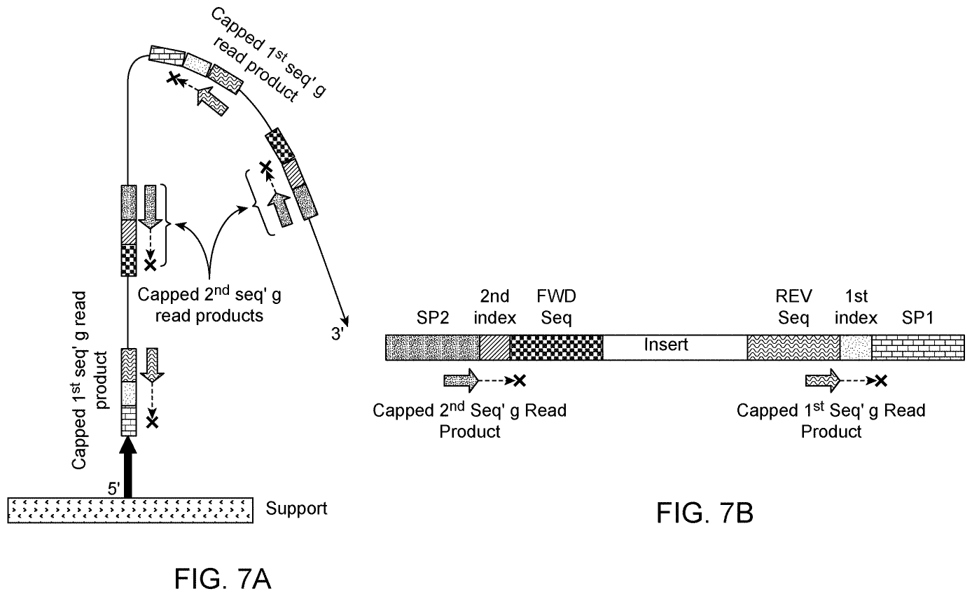

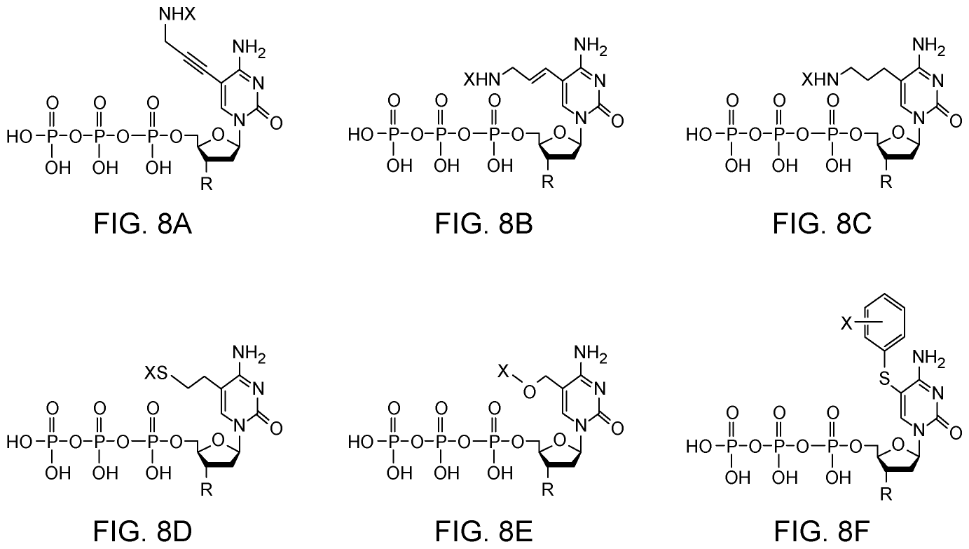

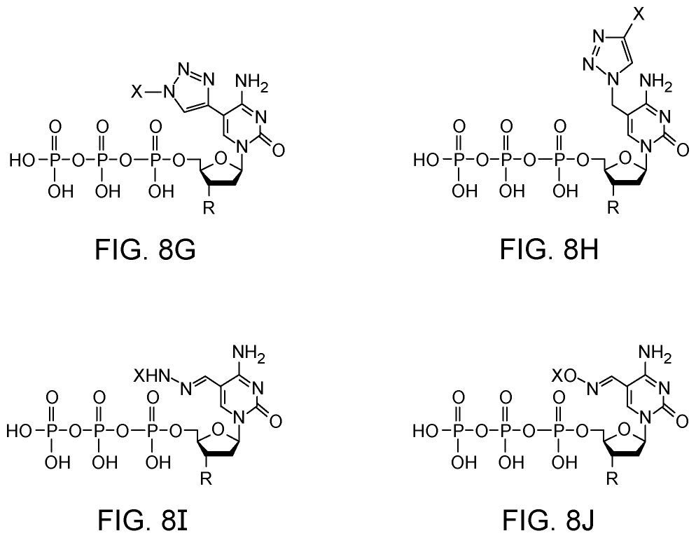

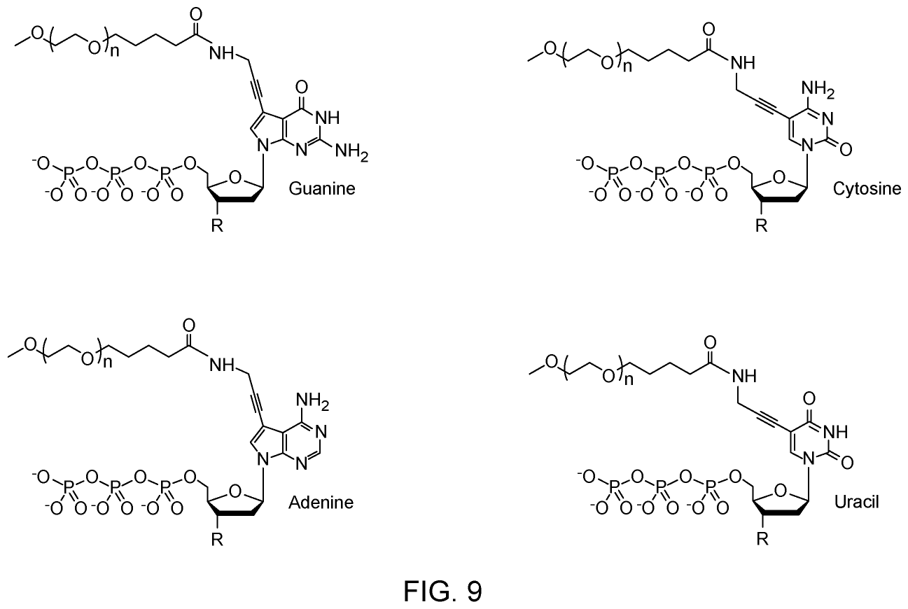

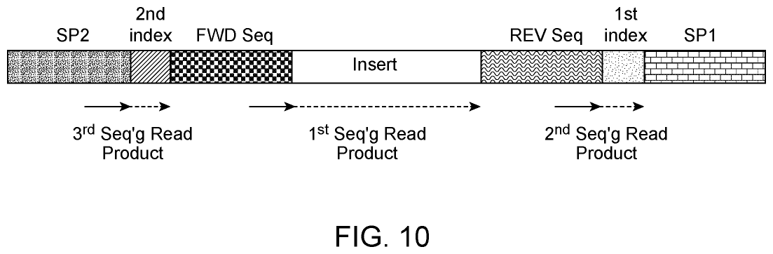

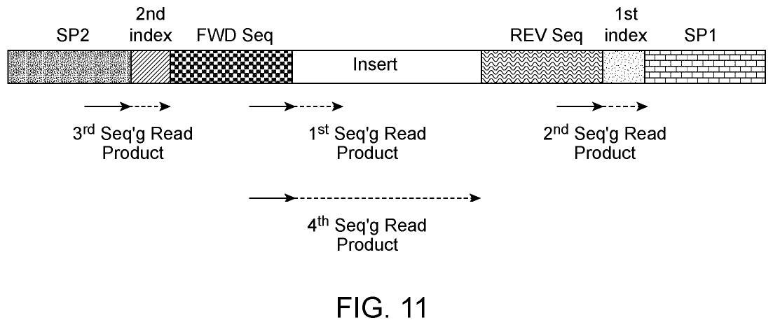

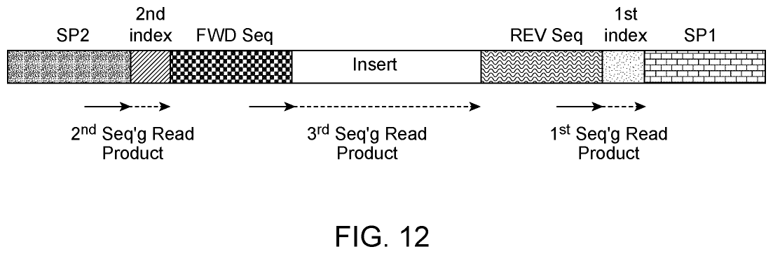

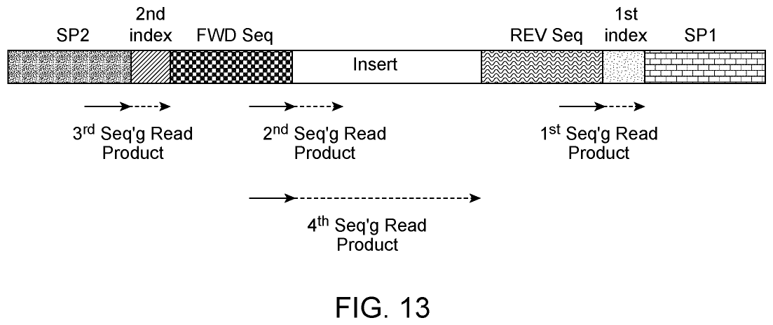

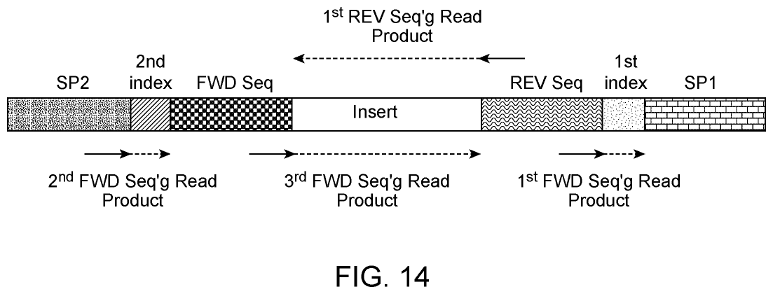

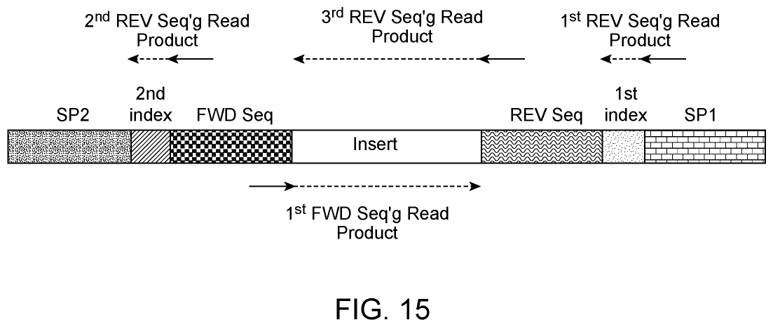

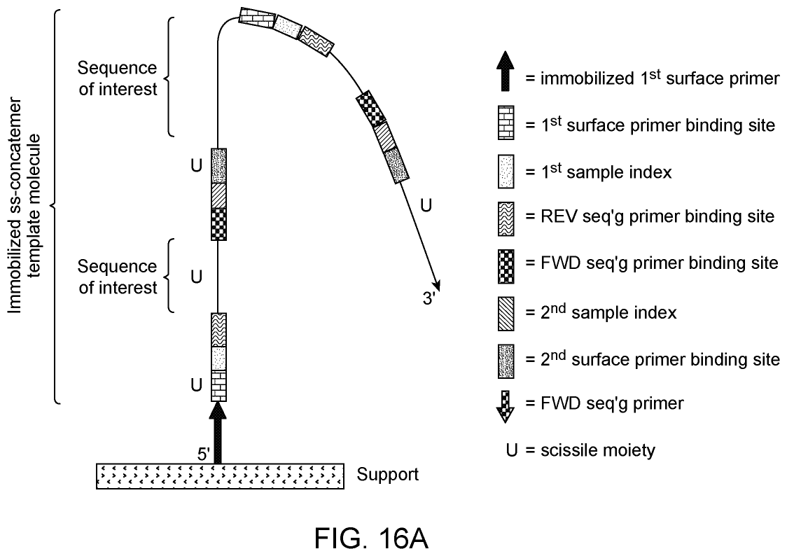

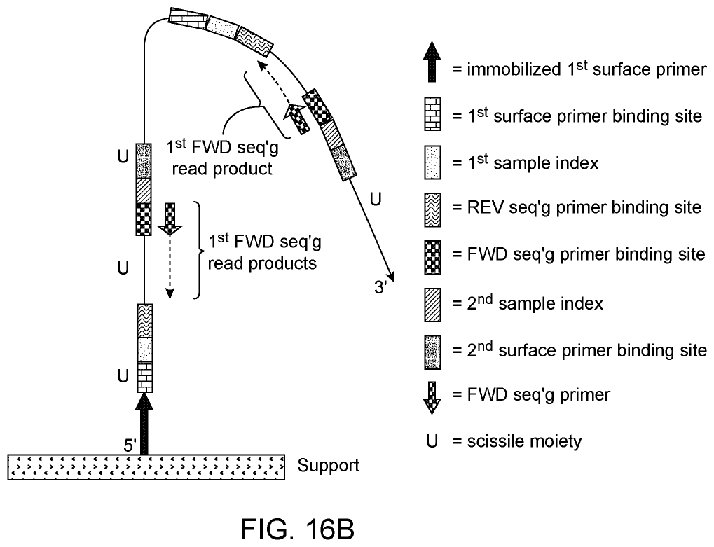

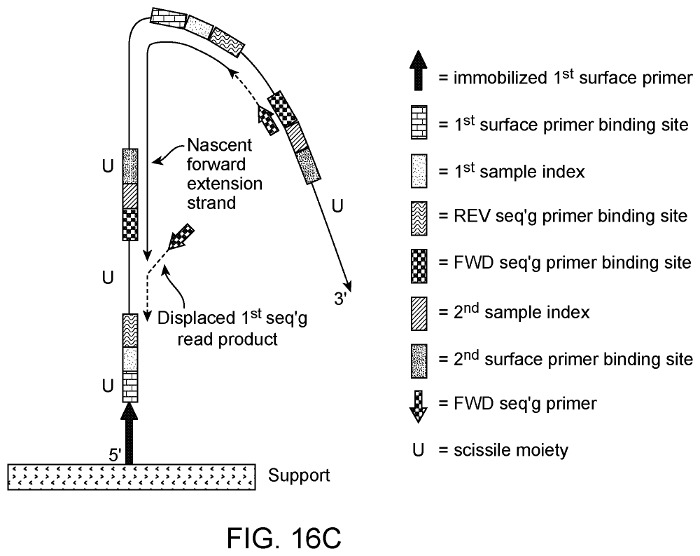

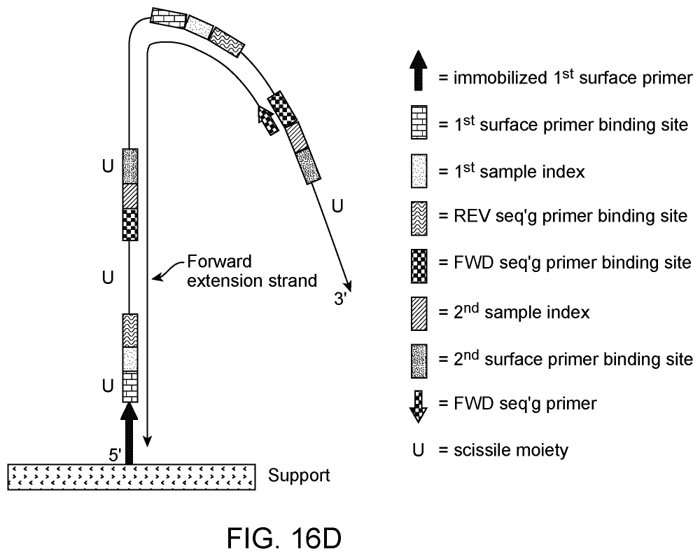

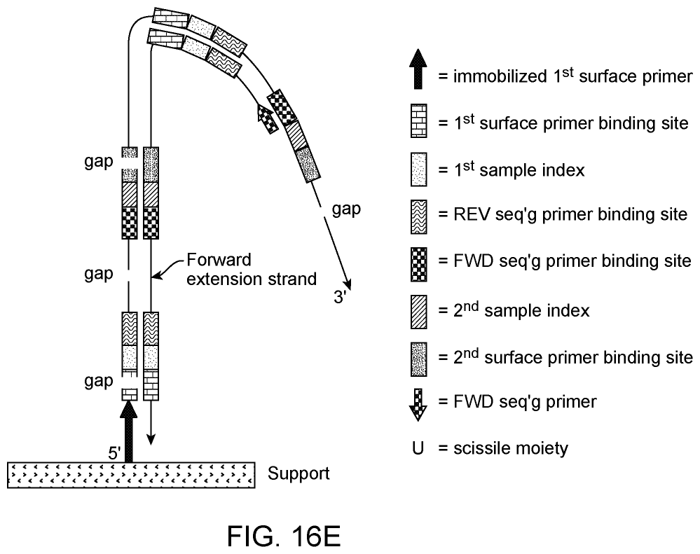

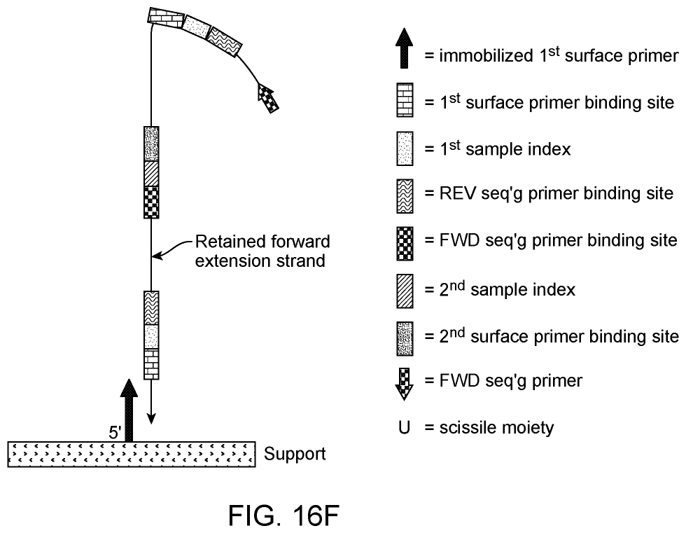

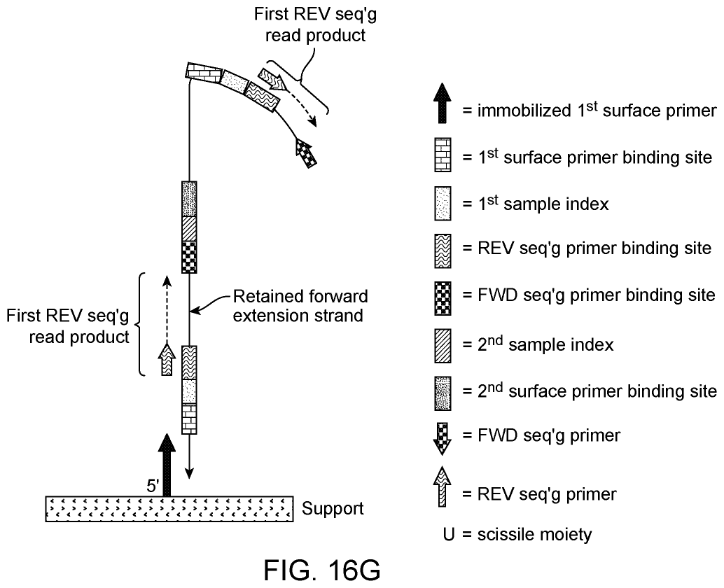

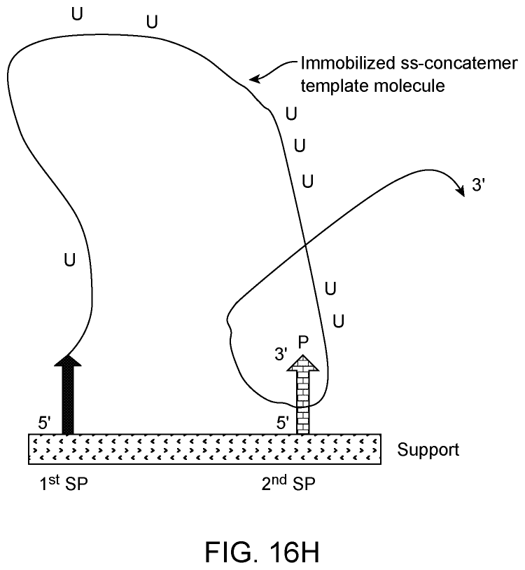

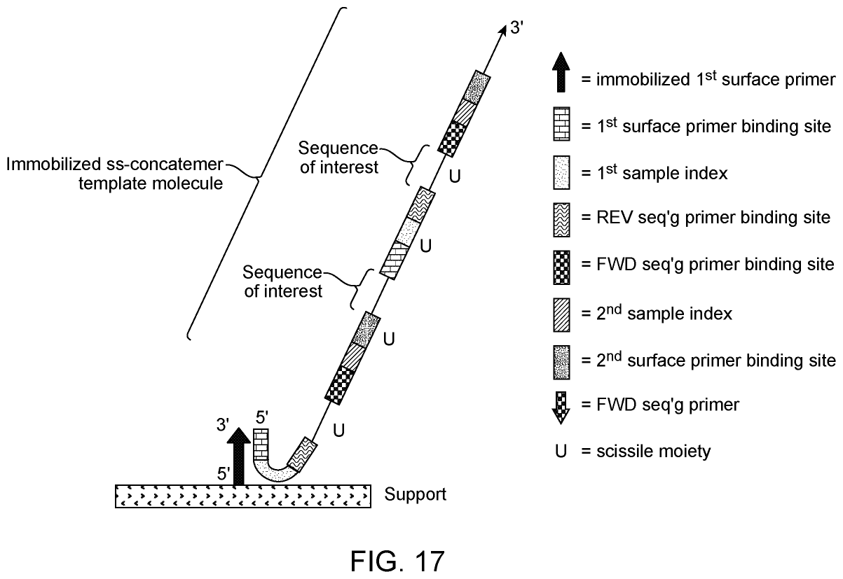

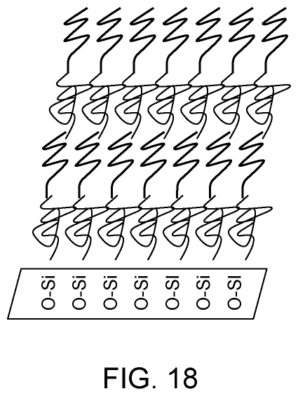

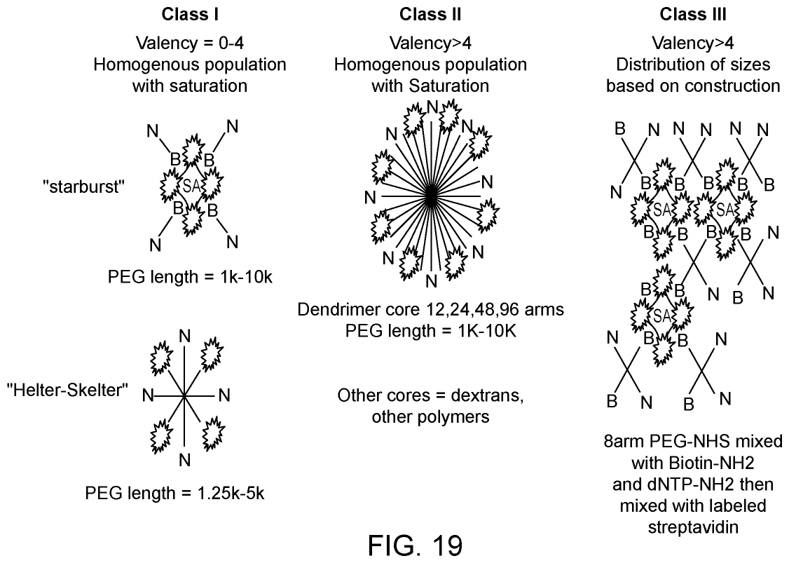

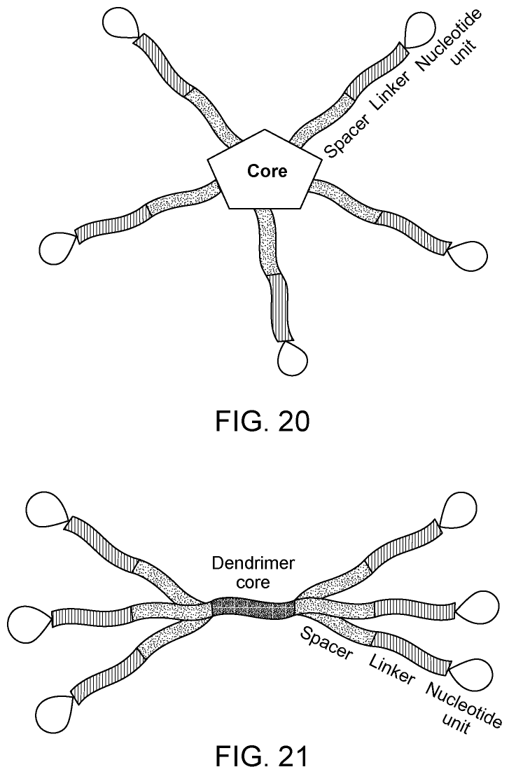

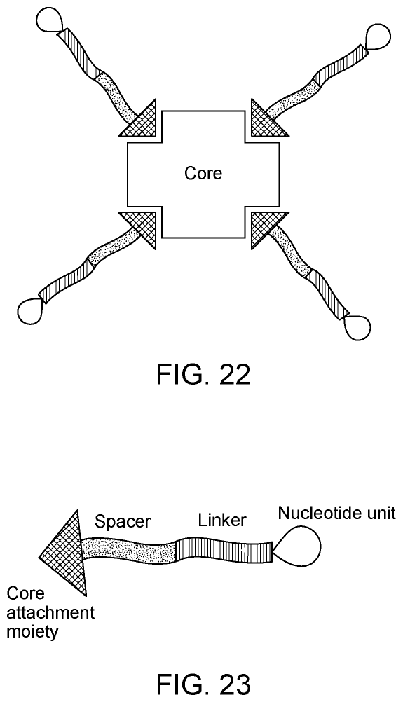

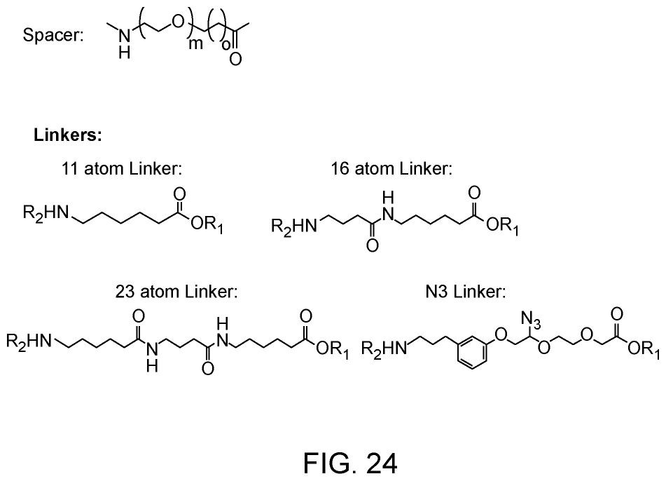

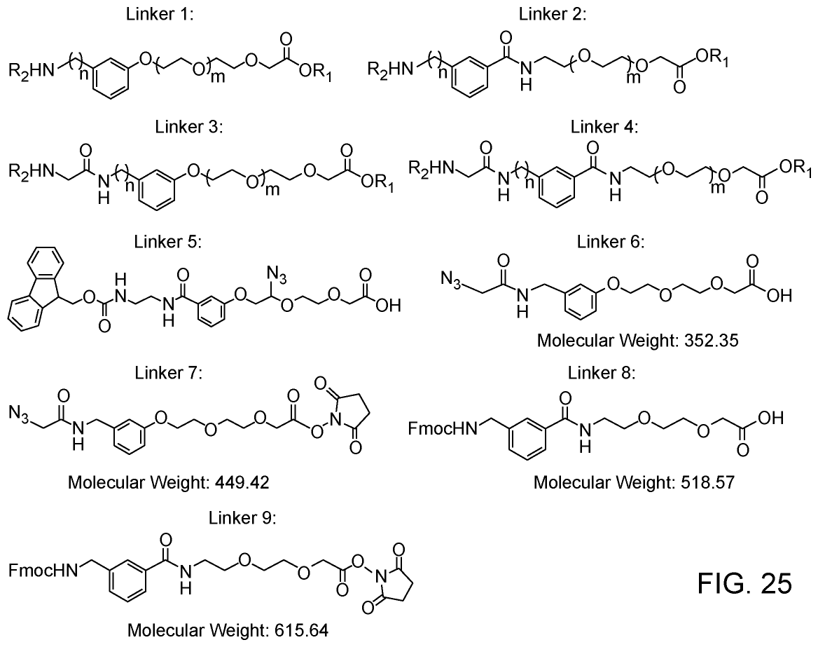

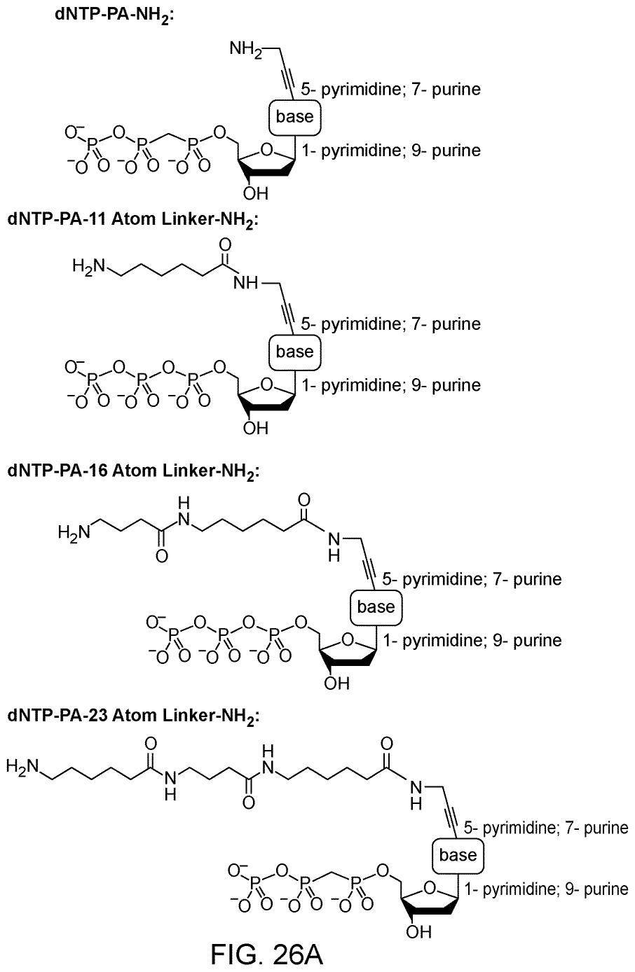

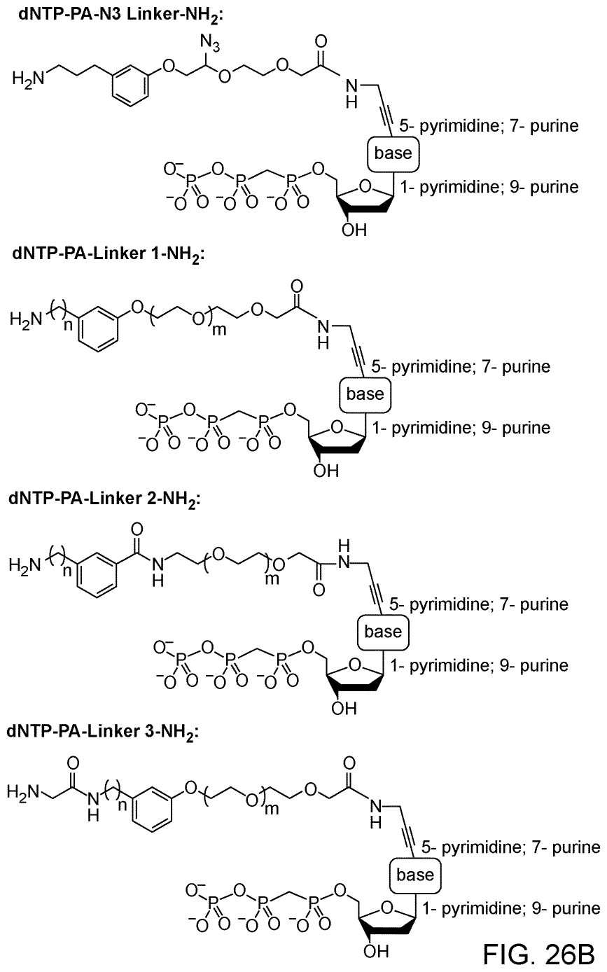

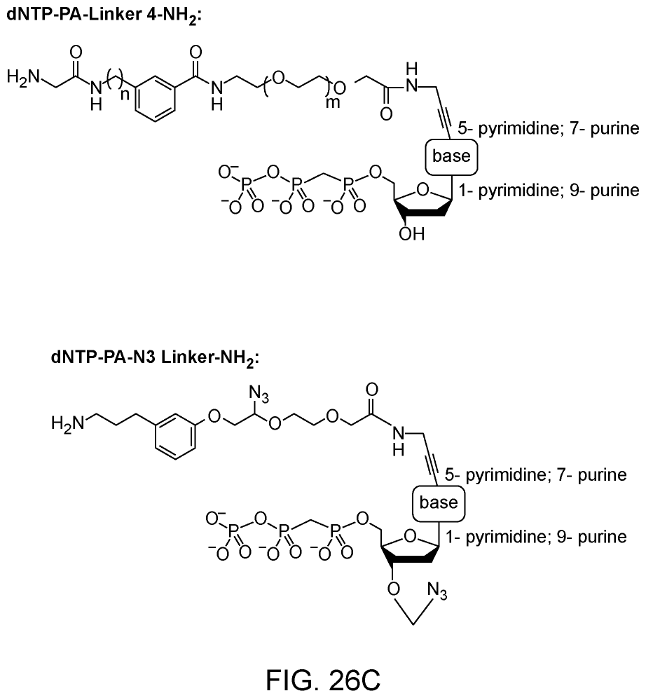

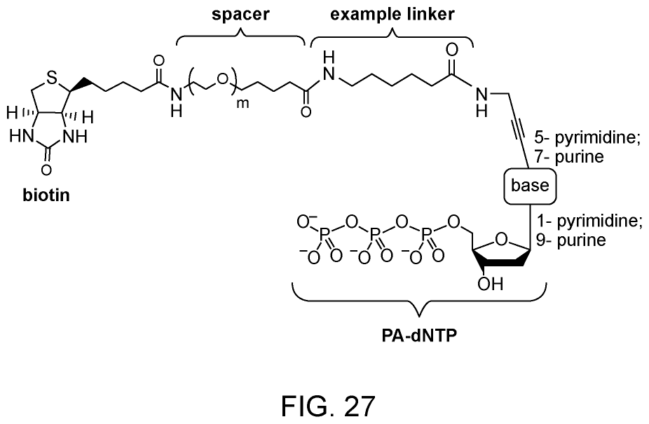

Alternatively, the concatemer template molecule can comprise at least one scissile moiety (e.g., uracil). The schematic in FIG. 3A also shows a first and second plurality of soluble sequencing primers that can hybridize to their respective universal sequencing primer binding sites on the template molecule. The schematics in FIGS. 3A, 4A, 5A, 6A and 7A show the workflow of sequencing the immobilized concatemer template molecule which is depicted in FIG. 3A, where the sequencing workflow employs read-capping nucleotide analogs. The legend for the various universal sequences can be found in FIG. 16A.

FIG. 3B is a schematic showing a linear template molecule having one copy an insert sequence (e.g., sequence-of-interest) and various universal sequences including universal primer binding sites (labelled FWD Seq and REV Seq). The linear template molecule can be immobilized to a support. The template molecule can lack a scissile moiety (e.g., uracil).

Alternatively, the template molecule can comprise at least one scissile moiety (e.g., uracil). The schematic in FIG. 3B also shows a first and second plurality of soluble sequencing primers that can hybridize to their respective universal sequencing primer binding sites on the template molecule. The schematics in FIGS. 3B, 4B, 5B, 6B and 7B show the workflow of sequencing the linear template molecule depicted in FIG. 3B, where the sequencing workflow employs read-capping nucleotide analogs. The legend for the various universal sequences can be found in FIG. 16A.

FIG. 4A is a schematic showing individual first sequencing primers hybridizing to their respective first universal sequencing primer binding sites on the nucleic acid concatemer template molecule, and sequencing the first region of the template molecule thereby generating a first plurality of sequencing read products.

FIG. 4B is a schematic showing a first sequencing primer hybridizing to a first universal sequencing primer binding site on the template molecule, and sequencing the first region of the nucleic acid concatemer template molecule thereby generating a first sequencing read product.

FIG. 5A is a schematic showing a first capping reaction which is conducted by incorporating a read-capping nucleotide analog into the terminal 3′ ends of individual first sequencing read products thereby generating a first plurality of capped sequencing read products. The read-capping nucleotide analog is depicted as a solid “X”.

FIG. 5B is a schematic showing a first capping reaction which is conducted by incorporating a read-capping nucleotide analog into the terminal 3′ end of the first sequencing read product thereby generating a first capped sequencing read product. The read-capping nucleotide analog is depicted as a solid “X”.

FIG. 6A is a schematic which shows retaining the plurality of capped first sequencing read products, which remain hybridized to the nucleic acid concatemer template molecule, and hybridizing individual second sequencing primers to their respective second universal sequencing primer binding sites on the concatemer template molecule, and sequencing the second region of the template molecule thereby generating a plurality of second sequencing read products.

FIG. 6B is a schematic which shows retaining the first capped sequencing read product which remains hybridized to the nucleic acid concatemer template molecule, and hybridizing a second sequencing primer to a second universal sequencing primer binding site on the template molecule, and sequencing the second region of the nucleic acid concatemer template molecule thereby generating a second sequencing read product.

FIG. 7A is a schematic showing a second capping reaction which is conducted by incorporating a read-capping nucleotide analog into the terminal 3′ ends of individual second sequencing read products thereby generating a plurality of capped second sequencing read products, and retaining the plurality of capped second sequencing read products which remain hybridized to the nucleic acid concatemer template molecule.

FIG. 7B is a schematic showing a second capping reaction which is conducted by incorporating a read-capping nucleotide analog into the terminal 3′ end of a second sequencing read product thereby generating a capped second sequencing read product, and retaining the capped second sequencing read product which remains hybridized to the nucleic acid concatemer template molecule.

FIGS. 8A-8J shows several embodiments of read-capping nucleotide analogs each comprising a heterocyclic base which is linked to a terminating moiety. FIG. 8A: the terminating moiety comprises a propargylamino moiety, where X can be a polymer (e.g., a PEG moiety). FIG. 8B: the terminating moiety comprises an allylamino moiety, where X can be a polymer (e.g., a PEG moiety). FIG. 8C: the terminating moiety comprises a propylamino moiety, where X can be a polymer (e.g., a PEG moiety). FIG. 8D: the terminating moiety comprises an ethylmercapto moiety, where X can be a polymer (e.g., a PEG moiety). FIG. 8E: the terminating moiety comprises a hydroxymethyl moiety, where X can be a polymer (e.g., a PEG moiety). FIG. 8F: the terminating moiety comprises an arylmercapto moiety, where X can be a polymer (e.g., a PEG moiety). FIG. 8G: the terminating moiety comprises a 1-X-1H-1,2,3-triazol-4-yl moiety, where X can be a polymer (e.g., a PEG moiety). FIG. 8H: the terminating moiety comprises a 5-X-1H-1,2,3-triazol-1-methyl moiety, where X can be a polymer (e.g., a PEG moiety). FIG. 8I: the terminating moiety comprises a hydrazone moiety, where X can be a polymer (e.g., a PEG moiety). FIG. 8J: the terminating moiety comprises an O-alkyl oxime moiety, where X can be a polymer (e.g., a PEG moiety).

FIG. 9 show several embodiments of read-capping nucleotide analogs each comprising a heterocyclic base which is linked to a terminating moiety. The terminating moiety comprises a polyethylene glycol (PEG) moiety or derivative thereof. In some embodiments, The PEG moiety comprises a molecular weight of about 1K Da-20 KDa (K Da: KiloDaltons).

FIG. 10 is a schematic showing an exemplary order of sequencing: Read 1 (e.g., full length sequencing the sequence-of-interest, “1^{st} Seq'g Read Product”); first sample index (“2^{nd} Seq'g Read Product”); and second sample index (“3^{rd} Seq'g Read Product”).

FIG. 11 is a schematic showing an exemplary order of sequencing: Read 1 (e.g., sequencing only a portion of the sequence-of-interest, “1^{st} Seq'g Read Product”); first sample index (“2^{nd} Seq'g Read Product”); second sample index; (“3^{rd} Seq'g Read Product”) and Read 1 (e.g., sequencing full length the sequence-of-interest, “4^{th} Seq'g Read Product”).

FIG. 12 is a schematic showing an exemplary order of sequencing: first sample index (“1^{st} Seq'g Read Product”); second sample index (“2^{nd} Seq'g Read Product”); Read 1 (e.g., full length sequencing the sequence-of-interest, “3^{rd} Seq'g Read Product”).

FIG. 13 is a schematic showing an exemplary order of sequencing: first sample index (“1^{st} Seq'g Read Product”); Read 1 (e.g., sequencing only a portion of the sequence-of-interest, “2^{nd} Seq'g Read Product”); second sample index (“3^{rd} Seq'g Read Product”); and Read 1 (e.g., full length sequencing the sequence-of-interest, “4^{th} Seq'g Read Product”).

FIG. 14 is a schematic showing an exemplary order of sequencing for a pairwise workflow: forward first sample index (“1^{st} FWD Seq'g Read Product”); forward second sample index (“2^{nd} FWD Seq'g Read Product”); forward Read 1 (e.g., full length sequencing the sequence-of-interest, “3^{rd} FWD Seq'g Read Product”); pairwise turn; reverse Read 2 (e.g., full length sequencing the sequence-of-interest, “1^{st} REV Seq'g Read Product”).

FIG. 15 is a schematic showing an exemplary order of sequencing for a pairwise workflow: forward Read 1 (e.g., full length sequencing the sequence-of-interest, “1^{st} FWD Seq'g Read Product”); pairwise turn; reverse first sample index (“1^{st} REV Seq'g Read Product”); reverse second sample index (“2^{nd} REV Seq'g Read Product”); and reverse Read 2 (e.g., full length sequencing the sequence-of-interest, “3^{rd} REV Seq'g Read Product”).

FIG. 16A is a schematic showing an exemplary single stranded nucleic acid concatemer template molecule immobilized to an immobilized first surface primer. The concatemer template molecule is covalently attached to the immobilized first surface primer. The immobilized concatemer template molecule comprises at least one nucleotide having a scissile moiety that can be cleaved to generate an abasic site in the immobilized concatemer template molecule. The immobilized concatemer template molecule can be generated by conducting an on-support rolling circle amplification reaction using a covalently closed circular library molecule carrying a universal adaptor sequence that can hybridize to an immobilized first surface primer. The arrangement of the sequence-of-interest and various universal adaptor sequences is for illustration purposes. The skilled artisan will appreciate that many other arrangements are possible. FIGS. 16B-16H show the workflow of pairwise sequencing the immobilized concatemer template molecule depicted in FIG. 16A.

FIG. 16B is a schematic showing an exemplary forward sequencing reaction conducted on the immobilized concatemer template molecule shown in FIG. 16A. The forward sequencing reaction can be conducted with a plurality of soluble forward sequencing primers and generates a plurality of first forward sequencing read products. The immobilized concatemer template molecule can comprise two or more first forward sequencing read products hybridized thereon.

FIG. 16C is a schematic showing an exemplary method for replacing the first forward sequencing read products by conducting a primer extension reaction with a strand displacing polymerase in the absence of an added soluble primer, thereby generating a forward extension strand. The strand displacing polymerase can use an upstream first forward sequencing read product to initiate a primer extension reaction.

FIG. 16D is a schematic showing an exemplary method for replacing the first forward sequencing read products by removing the first forward sequencing read products, and conducting a primer extension reaction with a new soluble forward sequencing primer thereby generating a forward extension strand.

FIG. 16E is a schematic showing an exemplary method for generating abasic sites in the immobilized single stranded concatemer template molecule at the nucleotides having the scissile moiety and generating gaps at the abasic sites to generate a plurality of gap-containing concatemer template molecules while retaining the forward extension strand and retaining the immobilized first surface primer. The forward extension strand can be generated for example by the methods depicted in FIG. 16C or 16D.

FIG. 16F is a schematic showing an exemplary retained forward extension strand after removal of the gap-containing concatemer template molecule as shown in FIG. 16E.

FIG. 16G is a schematic showing an exemplary reverse sequencing reaction conducted on the retained forward extension strand shown in FIG. 16F. The reverse sequencing reaction can be conducted with a plurality of soluble reverse sequencing primers. The retained forward extension strand can comprise two or more first reverse sequencing read products hybridized thereon. The first reverse sequencing read products are not hybridized to the first surface primer, or covalently joined to the first surface primer. Therefore, the first reverse sequencing read products are not immobilized to the support. For the sake of simplicity, FIGS. 16A-16G show an exemplary immobilized concatemer molecule with two copies of the sequence-of-interest and various universal primer binding sites. The skilled artisan will appreciate that the immobilized concatemer molecule can comprise more than two tandem copies of a unit where each unit comprises the sequence-of-interest and various universal primer binding sites.

FIG. 16H is a schematic showing an exemplary support having a first and second surface primers immobilized thereon. A portion of the immobilized concatemer template molecule shown in FIG. 16A is hybridized to the immobilized second surface primer. The immobilized second surface primers serve to pin down a portion of the immobilized concatemer template molecules to the support. The immobilized concatemer template molecule can have two or more copies of a universal binding sequence for an immobilized second surface primer. The portion of the immobilized concatemer template molecule that comprises the universal binding sequence for an immobilized second surface primer can hybridize to the immobilized second surface primer.

FIG. 17 is a schematic showing an exemplary single stranded nucleic acid concatemer template molecule immobilized to an immobilized first surface primer. The concatemer template molecule is hybridized to the immobilized first surface primer. The immobilized concatemer template molecule comprises at least one nucleotide having a scissile moiety that can be cleaved to generate an abasic site in the immobilized concatemer template molecule. In some embodiments, the immobilized concatemer template molecule can be generated by conducting an in-solution rolling circle amplification reaction and distributing the rolling circle amplification reaction onto the support. The arrangement of the sequence-of-interest and various universal adaptor sequences is for illustration purposes. The skilled artisan will appreciate that many other arrangements are possible. The immobilized concatemer template molecule can be subjected to the pairwise sequencing workflow that is depicted in FIGS. 16A-16G.

FIG. 18 is a schematic of an exemplary low binding support comprising a glass substrate and alternating layers of hydrophilic coatings which are covalently or non-covalently adhered to the glass, and which further comprises chemically-reactive functional groups that serve as attachment sites for oligonucleotide primers (e.g., capture oligonucleotides). In an alternative embodiment, the support can be made of any material such as glass, plastic or a polymer material.

FIG. 19 is a schematic of various exemplary configurations of multivalent molecules.

Left (Class I): schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’.

FIG. 20 is a schematic of an exemplary multivalent molecule comprising a generic core attached to a plurality of nucleotide-arms.

FIG. 21 is a schematic of an exemplary multivalent molecule comprising a dendrimer core attached to a plurality of nucleotide-arms.

FIG. 22 shows a schematic of an exemplary multivalent molecule comprising a core attached to a plurality of nucleotide-arms, where the nucleotide arms comprise biotin, spacer, linker and a nucleotide unit.

FIG. 23 is a schematic of an exemplary nucleotide-arm comprising a core attachment moiety, spacer, linker and nucleotide unit.

FIG. 24 shows the chemical structure of an exemplary spacer (top), and the chemical structures of various exemplary linkers, including an 11-atom Linker, 16-atom Linker, 23-atom Linker and an N3 Linker (bottom).

FIG. 25 shows the chemical structures of various exemplary linkers, including Linkers 1-9.

FIG. 26A shows the chemical structures of various exemplary linkers joined/attached to nucleotide units.

FIG. 26B shows the chemical structures of various exemplary linkers joined/attached to nucleotide units.

FIG. 26C shows the chemical structures of various exemplary linkers joined/attached to nucleotide units.

FIG. 27 shows the chemical structure of an exemplary biotinylated nucleotide-arm. In this example, the nucleotide unit is connected to the linker via a propargyl amine attachment at the 5 position of a pyrimidine base or the 7 position of a purine base.

### DETAILED DESCRIPTION

### Definitions The headings provided herein are not limitations of the various aspects of the disclosure, which aspects can be understood by reference to the specification as a whole.

Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to conventional methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those well-known and commonly used in the art.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the terms “about” and “approximately” refer to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. Also, where ranges and/or subranges of values are provided, the ranges and/or subranges can include the endpoints of the ranges and/or subranges.

The term “cellular biological sample” refers to a single cell, a plurality of cells, a tissue, an organ, an organism, or section of any of these cellular biological samples. The cellular biological sample can be extracted (e.g., biopsied) from an organism, or obtained from a cell culture grown in liquid or in a culture dish. The cellular biological sample comprises a sample that is fresh, frozen, fresh frozen, or archived (e.g., formalin-fixed paraffin-embedded; FFPE).

The cellular biological sample can be embedded in a wax, resin, epoxy or agar. The cellular biological sample can be fixed, for example in any one or any combination of two or more of acetone, ethanol, methanol, formaldehyde, paraformaldehyde-Triton or glutaraldehyde. The cellular biological sample can be sectioned or non-sectioned. The cellular biological sample can be stained, de-stained or non-stained.

The term “polymerase” and its variants, as used herein, refers to an enzyme comprising a domain that binds a nucleotide (or nucleoside) where the polymerase can form a complex having a template nucleic acid and a complementary nucleotide. The polymerase can have one or more activities including, but not limited to, base analog detection activities, DNA polymerization activity, reverse transcriptase activity, DNA binding, strand displacement activity, and nucleotide binding and recognition. A polymerase can be any enzyme that can catalyze polymerization of nucleotides (including analogs thereof) into a nucleic acid strand.

Typically but not necessarily such nucleotide polymerization can occur in a template-dependent fashion. Typically, a polymerase comprises one or more active sites at which nucleotide binding and/or catalysis of nucleotide polymerization can occur. In some embodiments, a polymerase includes other enzymatic activities, such as for example, 3′ to 5′ exonuclease activity or 5′ to 3′ exonuclease activity. In some embodiments, a polymerase has strand displacing activity. A polymerase can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze nucleotide polymerization (e.g., catalytically active fragment). The polymerase may be a catalytically inactive polymerase, catalytically active polymerase, reverse transcriptase, and another enzyme comprising a nucleotide binding domain. In some embodiments, a polymerase can be isolated from a cell, or generated using recombinant DNA technology or chemical synthesis methods. In some embodiments, a polymerase can be expressed in prokaryote, eukaryote, viral, or phage organisms. In some embodiments, a polymerase can be post-translationally modified proteins or fragments thereof. A polymerase can be derived from a prokaryote, eukaryote, virus or phage. A polymerase comprises DNA-directed DNA polymerase and RNA-directed DNA polymerase.

Exemplary polymerases are described, for example in U.S. Pat. No. 11,859,241, the contents of which are incorporated by reference in their entirety herein.

The term “strand displacing” refers to the ability of a polymerase to locally separate strands of double-stranded nucleic acids and synthesize a new strand in a template-based manner. Strand displacing polymerases displace a complementary strand from a template strand and catalyze new strand synthesis. During synthesis, nucleotides complementary to the template strand can be incorporated into the 3′ end of the new strand. Strand displacing polymerases include mesophilic and thermophilic polymerases. Strand displacing polymerases include wild type enzymes, and variants including exonuclease minus mutants, mutant versions, chimeric enzymes and truncated enzymes. Examples of strand displacing polymerases include phi29 DNA polymerase, large fragment of Bst DNA polymerase, large fragment of Bsu DNA polymerase (exo-), Bca DNA polymerase (exo-), Klenow fragment of E. coli DNA polymerase, T5 polymerase, M-MuL V reverse transcriptase, HIV viral reverse transcriptase, Deep Vent® DNA polymerase and KOD DNA polymerase. The phi29 DNA polymerase can be wild type phi29 DNA polymerase (e.g., MagniPhi™ from Expedeon), or variant EquiPhi29™ DNA polymerase (e.g., from ThermoFisher Scientific®), or chimeric QualiPhi™ DNA polymerase (e.g., from 4basebio).

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids can be isolated. Nucleic acids include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and chimeric forms containing DNA and RNA. Nucleic acids can be single-stranded (ss) or double-stranded (ds). Nucleic acids comprise polymers of nucleotides, where the nucleotides include natural or non-natural bases and/or sugars. Nucleic acids comprise naturally-occurring internucleosidic linkages, for example phosphodiester linkages. Nucleic acids comprise non-natural internucleoside linkages, including phosphorothioate, phosphorothiolate, or peptide nucleic acid (PNA) linkages. In some embodiments, nucleic acids comprise a one type of polynucleotides or a mixture of two or more different types of polynucleotides.

The nucleic acids of interest can be extracted from cells or cellular biological samples using any of a number of techniques known to those of skill in the art. For example, a typical DNA extraction procedure comprises (i) collection of the cell sample or tissue sample from which DNA is to be extracted, (ii) disruption of cell membranes (i.e., cell lysis) to release DNA and other cytoplasmic components, (iii) treatment of the lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis. A variety of suitable commercial nucleic acid extraction and purification kits are consistent with the disclosure herein. Examples include, but are not limited to, the QIAamp® kits (for isolation of genomic DNA from human samples) and DNAeasy® kits (for isolation of genomic DNA from animal or plant samples) from Qiagen (Germantown, MD), or the Maxwell® and ReliaPrep™ series of kits from Promega (Madison, WI).

The term “nucleotides” and related terms refers to a molecule comprising an heterocyclic base, a five carbon sugar (e.g., ribose or deoxyribose), and at least one phosphate group. Canonical or non-canonical nucleotides are consistent with use of the term. In some embodiments, the nucleotide comprises a monophosphate, diphosphate, or triphosphate, or corresponding phosphate analog. The term “nucleoside” refers to a molecule comprising an aromatic base and a sugar. Nucleotides and nucleosides can be non-labeled or labeled with a detectable reporter moiety.

Nucleotides (and nucleosides) typically comprise a heterocyclic base including substituted or unsubstituted nitrogen-containing parent heteroaromatic ring which are commonly found in nucleic acids, including naturally-occurring, substituted, modified, or engineered variants. The base of a nucleotide (or nucleoside) is capable of forming Watson-Crick and/or Hoogstein hydrogen bonds with an appropriate complementary base. Exemplary bases include, but are not limited to, purines and pyrimidines such as: 2-aminopurine; 2,6-diaminopurine; adenine (A); ethenoadenine, N⁶-Δ²-isopentenyladenine (6iA); N⁶-Δ²-isopentenyl-2-methylthioadenine (2ms6iA); N⁶-methyladenine; guanine (G), isoguanine; N²-dimethylguanine (dmG); 7-methylguanine (7mG); 2-thiopyrimidine; 6-thioguanine (6sG); hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional exemplary bases can be found in Fasman, 1989, in “Practical Handbook of Biochemistry and Molecular Biology”, pp. 385-394, CRC Press, Boca Raton, Fla.

Nucleotides (and nucleosides) typically comprise a sugar moiety, such as carbocyclic moiety (Ferraro and Gotor 2000 Chem. Rev. 100: 4319-48), acyclic moieties (Martinez, et al., 1999 Nucleic Acids Research 27: 1271-1274; Martinez, et al., 1997 Bioorganic & Medicinal Chemistry Letters vol. 7: 3013-3016), and other sugar moieties (Joeng, et al., 1993 J. Med. Chem. 36: 2627-2638; Kim, et al., 1993 J. Med. Chem. 36: 30-7; Eschenmosser 1999 Science 284:2118-2124; and U.S. Pat. No. 5,558,991). The sugar moiety comprises: ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, nucleotides comprise a chain of one, two or three phosphorus atoms where the chain is typically attached to the 5′ carbon of the sugar moiety via an ester or phosphoramide linkage. In some embodiments, the nucleotide is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

The term “operably linked” and “operably joined” or related terms as used herein refers to juxtaposition of components (i.e., adjacent components). The juxtapositioned components can be linked together covalently or non-covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, linkage between a primer binding sequence and a sequence of interest forms a nucleic acid library molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. The skilled artisan will appreciate that components may be operably linked, but need not be directly physically linked.

The terms “linked”, “joined”, “attached”, “appended” and variants thereof comprise any type of fusion, bond, adherence or association between any combination of compounds or molecules that is of sufficient stability to withstand use in the particular procedure. The procedure can include but are not limited to: nucleotide binding; nucleotide incorporation; de-blocking (e.g., removal of chain-terminating moiety); washing; removing; flowing; detecting; imaging and/or identifying. Such linkage can comprise, for example, covalent, ionic, hydrogen, dipole-dipole, hydrophilic, hydrophobic, or affinity bonding, bonds or associations involving van der Waals forces, mechanical bonding, and the like. In some embodiments, such linkage occurs intramolecularly, for example linking together the ends of a single-stranded or double-stranded linear nucleic acid molecule to form a circular molecule. In some embodiments, such linkage can occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages can be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998).

The term “primer” and related terms used herein refers to an oligonucleotide that is capable of hybridizing with a DNA and/or RNA polynucleotide template to form a duplex molecule. Primers can be single-stranded along their entire length or comprise single-stranded and double-stranded portions. Primers can comprise natural nucleotides and/or nucleotide analogs. Primers can be recombinant nucleic acid molecules or synthetic nucleic acid molecules. Primers may have any length, but typically range from 4-50 nucleotides. A typical primer comprises a 5′ end and 3′ end. The 3′ end of the primer can comprise a 3′ OH moiety which serves as a nucleotide polymerization initiation site in a polymerase-catalyzed primer extension reaction. Alternatively, the 3′ end of the primer can lack a 3′ OH moiety, or can comprise a terminal 3′ blocking group that inhibits nucleotide polymerization in a polymerase-catalyzed reaction. Any one nucleotide, or more than one nucleotide, along the length of the primer can be labeled with a detectable reporter moiety. A primer can be in solution (e.g., a soluble primer) or can be immobilized to a support (e.g., a capture primer).

The term “template nucleic acid”, “template polynucleotide”, “target nucleic acid” “target polynucleotide”, “template strand” and other variations refer to a nucleic acid strand that serves as the basis nucleic acid molecule for any of the amplification and/or sequencing methods describe herein. The template nucleic acid can be single-stranded or double-stranded, or the template nucleic acid can comprise single-stranded or double-stranded portions. The template nucleic acid can be obtained from a naturally-occurring source, recombinant form, or chemically synthesized to include any type of nucleic acid analog. The template nucleic acid can be linear, concatemeric, circular, or other forms.

The term “adaptor” and related terms refer to oligonucleotides that can be operably linked (appended) to a target polynucleotide, where the adaptor confers a function to the co-joined adaptor-target molecule. Adaptors can comprise DNA, RNA, chimeric DNA/RNA, or analogs thereof. Adaptors can comprise at least one ribonucleoside residue. Adaptors can be single-stranded, double-stranded, or comprise single-stranded and/or double-stranded portions. Adaptors can be configured to be linear, stem-looped, hairpin, or Y-shaped forms. Adaptors can be any length, including 4-100 nucleotides or longer. Adaptors can have blunt ends, overhang ends, or a combination of both. Overhang ends include 5′ overhang and 3′ overhang ends. The 5′ end of a single-stranded adaptor, or one strand of a double-stranded adaptor, can comprise a 5′ phosphate group or lack a 5′ phosphate group. Adaptors can comprise a 5′ tail that does not hybridize to a target polynucleotide (e.g., tailed adaptor), or adaptors can be non-tailed. At least a portion of the adaptors can comprise a known and pre-determined sequence. An adaptor can comprise a sequence that is complementary to at least a portion of a primer, such as an amplification primer, a sequencing primer, or a capture primer (e.g., soluble or immobilized capture primers). Adaptors can comprise a random sequence or degenerate sequence. Adaptors can comprise at least one inosine residue. Adaptors can comprise at least one phosphorothioate, phosphorothiolate and/or phosphoramidate linkage. Adaptors can comprise at least one barcode sequence which can be used to distinguish polynucleotides (e.g., insert sequences) from different sample sources in a multiplex assay. Adaptors can comprise at least one unique identification sequence (e.g., a molecular tag) that can be used to uniquely identify a nucleic acid molecule to which the adaptor is appended. In some embodiments, the unique identification sequence comprises 2-12 or more nucleotides having a known sequence. For example, the unique identification sequence comprises a known random sequence where a nucleotide at each position is randomly selected from nucleotides having a base A, G, C, T or U. Adaptors can comprise at least one restriction enzyme recognition sequence, including any one or any combination of two or more selected from a group consisting of type I, type II, type III, type IV, type Hs or type IIB.

The term “universal sequence” and related terms refers to a sequence in a nucleic acid molecule that is common among two or more polynucleotide molecules. For example, an adaptor having a universal sequence can be operably joined to a plurality of polynucleotides so that the population of co-joined molecules carry the same universal adaptor sequence. Examples of universal adaptor sequences include an amplification primer sequence, a sequencing primer sequence or a capture primer sequence (e.g., soluble or immobilized capture primers).

When used in reference to nucleic acid molecules, the terms “hybridize” or “hybridizing” or “hybridization” or other related terms refers to hydrogen bonding between two different nucleic acids to form a duplex nucleic acid. Hybridization also includes hydrogen bonding between two different regions of a single nucleic acid molecule to form a self-hybridizing molecule having a duplex region. Hybridization can comprise Watson-Crick or Hoogstein binding to form a duplex double-stranded nucleic acid, or a double-stranded region within a nucleic acid molecule. The double-stranded nucleic acid, or the two different regions of a single nucleic acid, may be wholly complementary, or partially complementary.

Complementary nucleic acid strands need not hybridize with each other across their entire length. The complementary base pairing can be the standard A-T or C-G base pairing, or can be other forms of base-pairing interactions. Duplex nucleic acids can comprise mismatched base-paired nucleotides.

When used in reference to nucleic acids, the terms “extend”, “extending”, “extension” and other variants, refers to incorporation of one or more nucleotides into a nucleic acid molecule. Nucleotide incorporation comprises polymerization of one or more nucleotides into the terminal 3′ OH end of a nucleic acid strand, resulting in extension of the nucleic acid strand. Nucleotide incorporation can be conducted with natural nucleotides and/or nucleotide analogs. Typically, but not necessarily, nucleotide incorporation occurs in a template-dependent fashion. Any suitable method of extending a nucleic acid molecule may be used, including primer extension catalyzed by a DNA polymerase or RNA polymerase.

The term “rolling circle amplification” or “RCA” generally refers to an amplification method that employs a circularized nucleic acid template molecule containing a target sequence of interest, an amplification primer binding sequence, and optionally one or more adaptor sequences such as a sequencing primer binding site and/or a sample index sequence. The rolling circle amplification reaction can be conducted under isothermal amplification conditions, and includes the circularized nucleic acid template molecule, an amplification primer, a strand-displacing polymerase and a plurality of nucleotides, to generate a concatemer molecule containing tandem repeat sequences of the circular template molecule including any adaptor sequences present in the original circularized nucleic acid template molecule. The concatemer template molecule can self-collapse to form a nucleic acid nanoball. The shape and size of the nanoball can be further compacted by including a pair of inverted repeat sequences in the circular template molecule, or by conducting the rolling circle amplification reaction with one or more compaction oligonucleotides. One of the advantages of using rolling circle amplification to generate clonal amplicons for a sequencing workflow is that the repeat copies of the target sequence in the nanoball can be simultaneously sequenced to increase signal intensity. In some embodiments, the rolling circle amplification reaction can be conducted in the presence of a plurality of compaction oligonucleotides having at least four consecutive guanines. The rolling circle amplification reaction generates concatemer template molecules comprising repeat copies of the universal binding sequence for the compaction oligonucleotide. At least one compaction oligonucleotide can form a guanine tetrad and hybridize to the universal binding sequences for the compaction oligonucleotide, and the resulting concatemer template molecule can fold to form an intramolecular G-quadruplex structure. The concatemer template molecules can self-collapse to form compact nanoballs. Formation of the guanine tetrads and G-quadruplexes in the nanoballs may increase the stability of the nanoballs to retain their compact size and shape which can withstand repeated flows of reagents for conducting any of the sequencing workflows described herein.

When used in reference to nucleic acids, the terms “amplify”, “amplifying”, “amplification”, and other related terms include producing multiple copies of an original polynucleotide template molecule, where the copies comprise a sequence that is complementary to the template sequence, and/or the copies comprise a sequence that is the same as the template sequence. In some embodiments, the copies comprise a sequence that is substantially identical to a template sequence, and/or is substantially identical to a sequence that is complementary to the template sequence.

The term “reporter moiety”, “reporter moieties” or related terms refers to a compound that generates, or causes to generate, a detectable signal. A reporter moiety is sometimes called a “label.” Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other. It is well known to one skilled in the art to select reporter moieties so that each absorbs excitation radiation and/or emits fluorescence at a wavelength distinguishable from the other reporter moieties to permit monitoring the presence of different reporter moieties in the same reaction or in different reactions. Two or more different reporter moieties can be selected having spectrally distinct emission profiles, or having minimal overlapping spectral emission profiles. Reporter moieties can be linked (e.g., operably linked) to nucleotides, nucleosides, nucleic acids, enzymes (e.g., polymerases or reverse transcriptases), or support (e.g., surfaces).

A reporter moiety (or label) can comprise a fluorescent label or a fluorophore. Exemplary fluorescent moieties which may serve as fluorescent labels or fluorophores include, but are not limited to, fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY® and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue® and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy3, Cy5, lanthanide chelates and derivatives such as BCPDA, TBP, TMT, BHHCT, BCOT, Europium chelates, Terbium chelates, Alexa Fluor® dyes, DyLight® dyes, Atto™ dyes, LightCycler® Red dyes, CALFluor dyes, JOE and derivatives thereof, Oregon Green™ dyes, WellRED dyes, IRD dyes, phycoerythrin and phycobilin dyes, Malachite green, stilbene, DEG dyes, NR dyes, near-infrared dyes and others known in the art such as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, or derivatives thereof, or any combination thereof. Cyanine dyes may exist in either sulfonated or non-sulfonated forms, and may consist of two indolenin, benzo-indolium, pyridium, thiozolium, and/or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-indolin-2-ylidene)penta-1,3-dien-1-yl)-3,3-dimethyl-3H-indol-1-ium or 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-sulfoindolin-2-ylidene)penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene)hepta-1,3,5-trien-1-yl]-3H-indolium or 1-(5-carboxypentyl)-2-[(1E,3E,5E,7Z)-7-(1-ethyl-5-sulfo-1,3-dihydro-2H-indol-2-ylidene)hepta-1,3,5-trien-1-yl]-3H-indolium-5-sulfonate), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule. Additional dyes are described, for example in U.S. Patent Application Publication No. 2024/0240249, the contents of which are incorporated by reference in their entirety herein.

In some embodiments, the reporter moiety can be a fluorescence resonance energy transfer (FRET) pair, such that multiple classifications can be performed under a single excitation and imaging step. As used herein, FRET may comprise excitation exchange (Forster) transfers, or electron-exchange (Dexter) transfers.

The term “persistence time” and related terms refers to the length of time that a binding complex remains stable without dissociation of any of the components. An exemplary binding complex comprises a nucleic acid template and nucleic acid primer, a polymerase, a nucleotide unit of a multivalent molecule or a free (e.g., unconjugated) nucleotide. The nucleotide unit or the free nucleotide can be complementary or non-complementary to a nucleotide residue in the template molecule. The nucleotide unit or the free nucleotide can bind to the 3′ end of the nucleic acid primer at a position that is opposite a complementary nucleotide residue in the nucleic acid template molecule. The persistence time is indicative of the stability of the binding complex and strength of the binding interactions. Persistence time can be measured by observing the onset and/or duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One exemplary label is a fluorescent label. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water.

The term “support” as used herein refers to a substrate that is designed for deposition of biological molecules or biological samples for assays and/or analyses. Examples of biological molecules to be deposited onto a support include nucleic acids (e.g., DNA, RNA), polypeptides, saccharides, lipids, a single cell or multiple cells. Examples of biological samples include but are not limited to saliva, phlegm, mucus, blood, plasma, serum, urine, stool, sweat, tears and fluids from tissues or organs. An exemplary support comprises the inner surface(s) of a capillary tube or a flowcell.

In some embodiments, the support is solid, semi-solid, or a combination of both. In some embodiments, the support is porous, semi-porous, non-porous, or any combination of porosity. In some embodiments, the support can be substantially planar, concave, convex, or any combination thereof. In some embodiments, the support can be cylindrical, for example comprising a capillary or interior surface of a capillary.

In some embodiments, the surface of the support can be substantially smooth. In some embodiments, the support can be regularly or irregularly textured, including bumps, etched, pores, three-dimensional scaffolds, or any combination thereof.

In some embodiments, the support comprises a bead having any shape, including spherical, hemi-spherical, cylindrical, barrel-shaped, toroidal, disc-shaped, rod-like, conical, triangular, cubical, polygonal, tubular or wire-like.

The support can be fabricated from any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

The present disclosure provides a plurality (e.g., two or more) of nucleic acid template molecules immobilized to (i.e. attached to) a support. In some embodiments, the immobilized plurality of nucleic acid template molecules have the same sequence or have different sequences. In some embodiments, individual nucleic acid template molecules in the plurality of nucleic acid template molecules are immobilized to different sites on the support. In some embodiments, two or more individual nucleic acid template molecules in the plurality of nucleic acid templates are immobilized to a site on the support.

The term “array” refers to a support comprising a plurality of sites located at pre-determined locations on the support to form an array of sites. The sites can be discrete and separated by interstitial regions. In some embodiments, the pre-determined sites on the support can be arranged in one dimension in a row or a column, or arranged in two dimensions in rows and columns. In some embodiments, the plurality of pre-determined sites is arranged on the support in an organized fashion. In some embodiments, the plurality of pre-determined sites is arranged in any organized pattern, including rectilinear, hexagonal patterns, grid patterns, patterns having reflective symmetry, patterns having rotational symmetry, or the like. The distance between different pairs of sites can be that same or can vary. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are located at pre-determined locations on the support. In some embodiments, a plurality of pre-determined sites on the support (e.g., 10²-10¹⁵ sites or more) comprise immobilized with nucleic acid template molecules to form a nucleic acid template array. In some embodiments, the nucleic acid template molecules are immobilized at a plurality of pre-determined sites by hybridization to immobilized surface capture primers. In some embodiments, the nucleic acid template molecules are immobilized at a plurality of pre-determined sites by covalent attachment to the surface capture primers. In some embodiments, the nucleic acid template molecules that are immobilized at a plurality of pre-determined sites, for example immobilized at 10²-10¹⁵ sites or more. In some embodiments, the immobilized nucleic acid template molecules are clonally-amplified to generate immobilized nucleic acid polonies at the plurality of pre-determined sites. In some embodiments, individual immobilized nucleic acid polonies comprise linear one copy molecules, or comprise single-stranded or double-stranded nucleic acid concatemer template molecules.

A support comprising a plurality of sites located at random locations on the support can be referred to herein as a support having randomly located sites thereon. The locations of the randomly located sites on the support are not pre-determined. The plurality of randomly located sites is arranged on the support in a disordered and/or unpredictable fashion. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are randomly located on the support. In some embodiments, a plurality of randomly located sites on the support (e.g., 10²-10¹⁵ sites or more) comprise immobilized with nucleic acid template molecules. In some embodiments, the nucleic acid template molecules are immobilized at a plurality of randomly located sites by hybridization to immobilized surface capture primers. In some embodiments, the nucleic acid template molecules are immobilized at a plurality of randomly located sites by covalent attachment to the surface capture primers. In some embodiments, the nucleic acid templates that are immobilized at a plurality of randomly located sites, for example immobilized at 10²-10¹⁵ sites or more. In some embodiments, the immobilized nucleic acid templates are clonally amplified to generate immobilized nucleic acid polonies at the plurality of randomly located sites. In some embodiments, individual immobilized nucleic acid polonies comprise linear molecules comprising a single copy of a sequence of interest and optionally adaptor or primer binding sequences. In alternative embodiments, individual immobilized nucleic acid polonies comprise linear molecules comprising single-stranded or double-stranded concatemer template molecules.

In some embodiments, the plurality of immobilized surface capture primers on the support (e.g., located at pre-determined or random locations on the support) are in fluid communication with each other to permit flowing a solution of reagents (e.g., nucleic acid template molecules, soluble primers, enzymes, nucleotides, divalent cations, buffers, and the like) onto the support so that the plurality of immobilized surface capture primers on the support can be essentially simultaneously reacted with the reagents in a massively parallel manner. In some embodiments, the fluid communication of the plurality of immobilized surface capture primers can be used to conduct nucleic acid amplification reactions (e.g., rolling circle amplification [RCA], multiple displacement amplification [MDA], polymerase chain reaction [PCR], bridge amplification and the like) essentially simultaneously on the plurality of immobilized surface capture primers. An exemplary support that allows flowing of a solution of reagents and fluid communication between various locations on the support is a flowcell.

In some embodiments, the plurality of immobilized nucleic acid polonies on the support are in fluid communication with each other to permit flowing a solution of reagents (e.g., enzymes, nucleotides, divalent cations, and the like) onto the support so that the plurality of immobilized nucleic acid polonies on the support can be essentially simultaneously reacted with the reagents in a massively parallel manner. In some embodiments, the fluid communication of the plurality of immobilized nucleic acid polonies can be used to conduct nucleotide binding assays and/or conduct nucleotide polymerization reactions (e.g., primer extension or sequencing) essentially simultaneously on the plurality of immobilized nucleic acid polonies, and optionally to conduct detection and imaging for massively parallel sequencing.

The terms “immobilized” or “immobilized to” and related terms refer to nucleic acid molecules that are attached to a support through covalent bond or non-covalent interaction, or attached to a coating on the support, or buried within a matrix formed by a coating on the support, where the nucleic acid molecules include surface capture primers, nucleic acid template molecules and extension products of capture primers. Extension products of capture primers includes nucleic acid concatemers (e.g., nucleic acid polonies). The nucleic acid molecules can be immobilized at pre-determined or random locations on the support. The nucleic acid molecules can be immobilized at pre-determined or random locations on or within a coating passivated on the support. In some cases, nucleic acid molecules can be immobilized to the support through hybridization to complementary sequences of other nucleic molecules that, in turn, are immobilized to the support.

The term “immobilized” and related terms can also refer to enzymes (e.g., polymerases) that are attached to a support through covalent bond or non-covalent interaction, or attached to a coating on the support, or buried within a matrix formed by a coating on the support. The enzymes can be immobilized at pre-determined or random locations on the support. The enzymes can be immobilized at pre-determined or random locations on or within a coating passivated on the support.

In some embodiments, one or more nucleic acid template molecules are immobilized on the support, for example immobilized at the sites on the support. In some embodiments, the one or more nucleic acid template molecules are clonally-amplified. In some embodiments, the one or more nucleic acid template molecules are clonally-amplified off the support (e.g., in-solution) and then deposited onto the support and immobilized on the support. In some embodiments, the clonal amplification reaction of the one or more nucleic acid template molecules is conducted on the support resulting in immobilization on the support. In some embodiments, the one or more nucleic acid template molecules are clonally-amplified (e.g., in solution or on the support) using a nucleic acid amplification reaction, including any one or any combination of: polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, and/or single-stranded binding (SSB) protein-dependent amplification.

The term “surface primer” and related terms refer to single-stranded oligonucleotides that are immobilized to a support and comprise a sequence that can hybridize to at least a portion of a nucleic acid template molecule. “Surface capture primers” can be used to immobilize template molecules to a support via hybridization. Surface capture primers can be immobilized to a support in a manner that resists primer removal during flowing, washing, aspirating, and changes in temperature, pH, salts, chemical and/or enzymatic conditions. Typically, but not necessarily, the 5′ end of a surface capture primer can be immobilized to a support or to a coating on the support (or embedded in a coating on the support). Alternatively, an interior portion or the 3′ end of a surface capture primer can be immobilized to a support. “Surface pinning primers” can be used to hybridize to one or more additional portions of a nucleic acid concatemer template molecule captured by a surface capture primer, and pin down a second (or further) portion of the nucleic acid template molecule on the support (see FIG. 16H). At least a portion of a surface pinning primer can hybridize to a portion of a nucleic acid concatemer template molecule to pin down a portion of the concatemer molecule to the support.

The sequence of surface capture primers can be wholly or partially complementary along their length to at least a portion of the nucleic acid template molecule. A support can comprise a plurality of immobilized surface capture primers having the same sequence, or having two or more different sequences. Surface capture primers can be any length, for example 4-50 nucleotides, or 50-100 nucleotides, or 100-150 nucleotides, or longer lengths.

A surface capture primer can have a terminal 3′ nucleotide having a sugar 3′ OH moiety which is extendible for nucleotide polymerization (e.g., polymerase catalyzed polymerization). A surface capture primer can have a terminal 3′ nucleotide having the 3′ sugar position linked to a chain-terminating moiety that inhibits nucleotide polymerization. The 3′ chain-terminating moiety can be removed (e.g., de-blocked) to convert the 3′ end to an extendible 3′ OH end using a de-blocking agent. Examples of chain terminating moieties include alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group, or any of the 3′ chain-terminating moieties described herein. Azide type chain terminating moieties including azide, azido and azidomethyl groups. Examples of de-blocking agents include a phosphine compound, such as Tris(2-carboxyethyl)phosphine (TCEP) and bis-sulfo triphenyl phosphine (BS-TPP), for chain-terminating groups azide, azido and azidomethyl groups. Examples of de-blocking agents include tetrakis(triphenylphosphine)palladium(0) (Pd(PPh₃)₄) with piperidine, or with 2,3-Dichloro-5,6-dicyano-1,4-benzo-quinone (DDQ), for chain-terminating groups alkyl, alkenyl, alkynyl and allyl. Examples of a de-blocking agent includes H2 Pd/C for chain-terminating groups benzyl. Examples of de-blocking agents include phosphine, beta-mercaptoethanol or dithiothritol (DTT), for chain-terminating groups amine, amide, keto, isocyanate, phosphate, thio and disulfide. Examples of de-blocking agents include potassium carbonate (K₂CO₃) in MeOH, triethylamine in pyridine, and Zn in acetic acid (AcOH), for carbonate chain-terminating groups. Examples of de-blocking agents include tetrabutylammonium fluoride, pyridine-HF, with ammonium fluoride, and triethylamine trihydrofluoride, for chain-terminating groups urea and silyl.

The term “sequencing” and related terms refer to a method for obtaining nucleotide sequence information from a nucleic acid molecule, typically by determining the identity of at least some nucleotides (including their nucleobase components) within the nucleic acid molecule, and their order in the nucleic acid molecule. In some embodiments, the sequence information of a given region of a nucleic acid molecule includes identifying each and every nucleotide within a region that is sequenced. In some embodiments, sequencing information determines only some of the nucleotides a region, while the identity of some nucleotides remains undetermined or incorrectly determined. Any suitable method of sequencing may be used. In an exemplary embodiment, sequencing can include label-free or ion based sequencing methods. In some embodiments, sequencing can include labeled or dye-containing nucleotide or fluorescent based nucleotide sequencing methods. In some embodiments, sequencing can include polony-based sequencing or bridge sequencing methods. In some embodiments, the sequencing employs polymerases and multivalent molecules for generating at least one avidity complex, wherein individual multivalent molecules comprise a plurality of nucleotide units tethered to a core. In some embodiments, the sequencing employs polymerases and free nucleotides for performing sequencing-by-synthesis. In some embodiments, the sequencing employs a ligase enzyme and a plurality of sequence-specific oligonucleotides for performing sequence-by-ligation.

### Introduction

The present disclosure provides compositions and methods for sequentially sequencing different regions of the same nucleic acid template molecule and reducing residual signals that contribute to poor sequencing quality. The provided compositions and methods can be employed for pairwise sequencing workflows. The provided compositions and methods can be employed for uniplex and multiplex sequencing workflows.

Traditional multiplex sequencing workflows require sequencing a first region (e.g., a sequence-of-interest region) using a first sequencing primer, and sequencing a second region (e.g., a sample index region) using a second sequencing primer. The first and second sequencing primers are designed to hybridize to different regions of the same template molecule for conducting separate sequencing reactions to generate separate reads of the sequence-of-interest and sample index(es) regions. The sequencing read products are typically generated by conducting polymerase-catalyzed primer extension reactions. The sequencing read products that are synthesized from sequencing the first region must be efficiently removed prior to sequencing the second region while leaving the template molecule undamaged. Traditional sequencing workflows that employ chemical-based reagents (e.g., NaOH and/or formamide) to denature the sequencing read products can be harsh, resulting in damage to the template molecule. Switching to gentler chemical-based denaturation conditions may be less damaging to the template molecule but do not effectively remove the sequencing read products which contribute to residual signals. After multiple sequencing cycles, the residual signals can accumulate making it difficult to image and detect true sequencing signals. Residual signals cause poor sequencing quality and reduce sequencing accuracy.

The order of sequencing the sequence-of-interest and the sample index(es) regions also presents a challenge. When the sequence-of-interest region is sequenced before the sample index(es), the longer sequencing read product of the sequence-of-interest region must be removed prior to sequencing the shorter sample index regions. Inefficient removal of the sequencing read product from the sequence-of-interest region in previous sequencing cycles will generate residual signals in subsequent sequencing cycles of the sample index region.

Accumulation of residual signals in subsequent sequencing cycles reduces sequencing accuracy which can lead to mis-alignment of sample indexes with their proper sequence-of-interest regions. This phenomenon can contribute to index hopping, when data from one sample associated with one sample index “hops” to a different sample index during sequencing.

Thus, efficient removal of a sequencing read product from previous sequencing cycles will improve the overall accuracy of sequencing data. High accuracy sequencing of different regions of the template molecule is dependent on many parameters, including high signal intensity, reduced residual signals from previous sequencing reads, and preserving intact template molecules after multiple sequencing cycles.

The present disclosure provides compositions comprising read-capping nucleotide analogs, and methods that employ the read-capping nucleotide analogs. In some embodiments, the read-capping nucleotide analogs can be employed for sequentially sequencing different regions of the same nucleic acid template molecule. The read-capping nucleotide analogs offer several advantages over the traditional chemical-based reagents that use formamide, alkaline conditions and/or elevated temperatures for denaturing/removing sequencing read products in a sequencing workflow. The read-capping nucleotide analogs do not require formamide or alkaline conditions. The read-capping workflow can be conducted at low temperatures that pose reduced risk of damaging the template molecules.

It is well known that formamide lowers the melting temperature of duplex DNA in a linear manner by about 2.4-2.9 °C per mole of formamide, or about 0.6 °C per percent formamide. The length of the duplexed region and the G+C composition also influences the melting temperature of duplexed DNA. A common chemical-based method for removing sequencing read products includes denaturation at 50-75 °C in the presence of 20-40% formamide. Denaturation of duplex DNA at high temperatures can cause strand breakage and depurination which can lead to loss of intact template molecules after multiple sequencing cycles, and reduced signal intensity. Additionally, formamide is classified as a reproductive toxic substance which can be inhaled, ingested or absorbed through the skin. Laboratory safety regulations dictate that disposal of any reagent containing formamide must be treated as a chemical waste.

By contrast, read-capping nucleotide analogs can be incorporated at the terminal 3′ end of a sequencing read product when sequencing a first region of a template molecule is completed, to generate a first capped sequencing read product. The first capped sequencing read products are not removed from the template molecules prior to sequencing a second region of the template molecules. Removal of the first capped sequencing read product from the template molecule is obviated because the read-capping nucleotide analog, once incorporated into the read product (the “incorporated read-capping nucleotide analog”) blocks binding and incorporation of nucleotide reagents used to sequence a second region of the nucleic acid template molecule.

Thus, the template molecules are not subjected to chemical-based de-hybridization reagents and high temperatures typically used to remove previously-generated sequencing read products.

Using the read-capping nucleotide analogs, without the need to remove the sequencing read products, reduces damage to the template molecules and preserves intact template molecules after numerous sequencing cycles. When the immobilized template molecules are concatemers that collapse to form nucleic acid nanoballs, sequencing methods that employ the read-capping nucleotide analogs can reduce unraveling of the nanoballs, and can retain the shape and size of the nanoballs, after numerous sequencing cycles. Thus, methods that employ the read-capping nucleotide analogs can improve the FWHM (full width half maximum) of a spot image of the nanoball after numerous sequencing cycles.

### Read-Capping Nucleotide Analogs

In one aspect, the present disclosure provides read-capping nucleotide analogs which comprise: (i) a heterocyclic base, (ii) a ribose sugar, and (iii) a polyphosphate chain, wherein the heterocyclic base is linked to a terminating moiety (e.g., a base-linked chain terminating moiety). In some embodiments, the present disclosure provides a read-capping nucleotide analog that can be incorporated into a terminal 3′ end of a primer in a polymerase-catalyzed primer extension reaction to generate a read-capped primer product. In some embodiments, the present disclosure provides read-capping nucleotide analogs that can be incorporated into the terminal 3′ end of a nascent primer, in a polymerase-catalyzed primer extension reaction to generate a capped primer product.

In some embodiments, the read-capping nucleotide analog is incorporated into the 3′ terminal end of a primer product. Once incorporated, the read-capping nucleotide analog can block polymerase-mediated binding of a nucleotide reagent to the terminal 3′ ends of the read capped primer product, where the nucleotide reagent comprises a multivalent molecule. In some embodiments, a multivalent molecule comprises a core attached to multiple nucleotide arms, and individual nucleotide arms are attached to a nucleotide unit. In some embodiments, a multivalent molecule comprises (1) a core; and (2) a plurality of nucleotide arms, each arm comprising (i) a core attachment moiety, (ii) a spacer comprising, (iii) a linker, and (iv) a nucleotide unit, wherein the core is attached to the plurality of nucleotide arms, wherein the spacer is attached to the linker, wherein the linker is attached to the nucleotide unit (e.g., see FIGS. 19-23).

In some embodiments, after incorporation into the 3′ terminal end of a primer product, the read-capping nucleotide analog can block polymerase-catalyzed incorporation of a nucleotide reagent into the terminal 3′ ends of the read capped primer product. The nucleotide reagent can comprise a canonical nucleotide or a chain terminating nucleotide. In some embodiments, the chain terminating nucleotide comprises (i) a heterocyclic base, (ii) a ribose sugar (e.g., ribose or deoxyribose), and (iii) a polyphosphate chain, where the 2′ or 3′ sugar position is linked to a chain terminating moiety by a cleavable or non-cleavable linker. Numerous chain terminating moieties are described below.

In another aspect, the present disclosure provides read-capping nucleotide analogs comprising: (i) a heterocyclic base, (ii) a ribose sugar, and (iii) at least one phosphate group, wherein the heterocyclic base is linked to a terminating moiety. In some embodiments, the terminating moiety is linked to the heterocyclic base by a linker that is non-cleavable or cleavable. In some embodiments, the read-capping nucleotide analogs can be non-labeled or labeled with a detectable reporter moiety. FIGS. 8A-8J and 9 show illustrative read-capping nucleotide analogs.

In some embodiments, the read-capping nucleotide analogs comprise a heterocyclic base comprising a substituted nitrogen-containing parent heteroaromatic ring, including naturally-occurring, substituted, modified, or engineered variants. In some embodiments, the read-capping nucleotide analogs comprise a heterocyclic base comprising a nitrogen-containing parent heteroaromatic ring, including naturally-occurring, substituted, modified, or engineered variants. The base of a read-capping nucleotide analog is capable of forming Watson-Crick and/or Hoogstein hydrogen bonds with an appropriate complementary base. Illustrative bases include, but are not limited to, purines and pyrimidines such as: 2-aminopurine; 2,6-diaminopurine; adenine (A); ethenoadenine; N⁶-Δ²-isopentenyladenine (6iA); N⁶-Δ²-isopentenyl-2-methylthioadenine (2ms6iA); N⁶-methyladenine; guanine (G); isoguanine; N²-dimethylguanine (dmG); 7-methylguanine (7mG); 2-thiopyrimidine; 6-thioguanine (6sG); hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional exemplary bases can be found in Fasman, 1989, in “Practical Handbook of Biochemistry and Molecular Biology”, pp. 385-394, CRC Press, Boca Raton, Fla.

In some embodiments, the read-capping nucleotide analogs comprise a base-linked terminating moiety which be linked to the nucleobase by an alkyl linkage. In some embodiments, the linkage comprises an alkyl group. In some embodiments, the linkage comprises an alkenyl group. In some embodiments, the linkage comprises an alkynyl group.

In some embodiments, the base-linked terminating moiety comprises a polymer moiety. In some embodiments, the base-linked terminating moiety comprises a polyether moiety. In some embodiments, the base-linked terminating moiety comprises polyethylene glycol, In some embodiments, the base-linked terminating moiety comprises polypropylene glycol In some embodiments, the base-linked terminating moiety comprises polyvinyl acetate. In some embodiments, the base-linked terminating moiety comprises polylactic acid. In some embodiments, the base-linked terminating moiety comprises polyglycolic acid. In some embodiments, the base-linked terminating moiety comprises a polyamide moiety. In some embodiments, the base-linked terminating moiety comprises a polyester moiety. In some embodiments, the base-linked terminating moiety comprises apoly(glycerol) moiety. In some embodiments, the base-linked terminating moiety comprises a poly(oxazoline) moiety. In some embodiments, the base-linked terminating moiety comprises a poly(hydroxypropyl methacrylate) moiety (PHPMA). In some embodiments, the base-linked terminating moiety comprises a poly(2-hydroxyethyl methacrylate) moiety (PHEMA). In some embodiments, the base-linked terminating moiety comprises a poly(N-(2-hydroxypropyl) methacrylamide) moiety (HPMA). In some embodiments, the base-linked terminating moiety comprises a poly(vinylpyrrolidone) moiety (PVP). In some embodiments, the base-linked terminating moiety comprises a poly(N,N-dimethyl acrylamide) moiety (PDMA). In some embodiments, the base-linked terminating moiety comprises a poly(N-acryloylmorpholine) moiety (PAcM). In some embodiments, the base-linked terminating moiety comprises a synthetic zwitterionic moiety such as a poly(carboxybetaine acrylamide) moiety, a poly(carboxybetaine methacrylate) moiety, a poly(sulfobetaine methacrylate) moiety or a poly(methacryloyloxyethyl phosphorylcholine) moiety. In some embodiments, the base-linked terminating moiety comprises a polyglutamate moiety. In some embodiments, the base-linked terminating moiety comprises a polyaspartate moiety. In some embodiments, the base-linked terminating moiety comprises a polylysine moiety. In some embodiments, the base-linked terminating moiety comprises a polyethyeleneimine moiety. In some embodiments, the base-linked terminating moiety comprises a polysialic acid moiety.

In some embodiments, the base-linked terminating moiety comprises a linker, for example an 11 atom linker, a 16 atom linker, a 23 atom linker or an N3 linker which are shown in FIG. 24. In some embodiments, the base-linked terminating moiety comprises a polymer linker, for example any one of Linkers 1-9 shown in FIG. 25.

In some embodiments, the base-linked terminating moiety comprises a propargylamino moiety, an allylamino moiety, a propylamino moiety, an ethylmercapto moiety, a hydroxymethyl moiety, an arylmercapto moiety (e.g., see FIGS. 8A-8F). In some embodiments, the base-linked terminating moiety comprises a 1-X-1H-1,2,3-triazol-4-yl moiety, a 5-X-1H-1,2,3-triazol-1-methyl moiety, a hydrazone moiety or an O-alkyl oxime moiety (e.g., see FIGS. 8G-8J). In some embodiments, the “X” in FIGS. 8A-8J comprises a polymer such as for example any polymer described above.

In some embodiments, the base-linked terminating moiety comprises a hydrophilic polymer of ethylene oxide. In some embodiments, the base-linked terminating moiety comprises a polyethylene glycol (PEG) moiety (e.g., H—(OCH₂CH₂)ₙ—OH. In some embodiments, the PEG moiety comprises a molecular weight of about 100-200 Da, 200-300 Da, 300-400 Da, 400-500 Da, 1K Da, 2K Da, 3K Da, 4K Da, 5K Da, 10K Da, 15 K Da, 20K Da, 30K Da, 40K Da, 50K Da, or larger molecular weight PEG. In some embodiments, the value of n can be 1, or at least 2, or at least 5, or at least 10, at least 20, at least 25, at least 50, at least 75, at least 100, at least 200, at least 500, at least 750, at least 1000, or 2000 or more (see, e.g., FIG. 9). In some embodiments, the PEG moiety comprises a linear PEG molecule. In some embodiments, the PEG moiety comprises a branched PEG molecule. In some embodiments, the branched PEG moiety molecule comprise 4-8 branches. In some embodiments, the terminating moiety comprises an aromatic group having a six-carbon ring structure. In some embodiments, the PEG moiety can be functionalized with a methoxy group (OCH₃), amino group (NH₂), carboxyl group (COOH) and/or hydroxyl group (OH). For example, the base-linked terminating moiety comprises polyethylene glycol methyl ether (mPEG) (e.g., CH₃(OCH₂CH₂)ₙ—OH). In some embodiments, the “X” in FIGS. 8A-8J comprises a PEG moiety or derivative thereof such as for example any PEG moiety described above.

In some embodiments, a set of read-capping nucleotide analogs comprises four types of read-capping nucleotide analogs, including dATP, dGTP, dCTP and dTTP/dUTP, wherein the nucleobase of each type of read-capping nucleotide analog is linked to the same type of terminating moiety. For example, the same type of PEG moiety is linked to the nucleobase of dATP, dGTP, dCTP and dTTP/dUTP.

In some embodiments, a set of read-capping nucleotide analogs comprises four types of read-capping nucleotide analogs, including dATP, dGTP, dCTP and dTTP/dUTP, wherein the nucleobases of at least two types of read-capping nucleotide analog are linked to a different terminating moiety. For example, at least one type of read-capping nucleotide analog (e.g., dATP) comprises a nucleobase that is linked to a PEG moiety that differs from the PEG moiety of other read-capping nucleotide analogs (e.g., dGTP, dCTP or dTTP/dUTP). In some embodiments, the PEG moiety can differ in length. In some embodiments, the PEG moiety can differ in molecular weight. In some embodiments, the nucleobases of one, two, three or four types of read-capping nucleotide analogs can be linked to a PEG moiety by a different linkage (e.g., a different alkyl, alkenyl, or alkynyl linkage). In some embodiments, the nucleobases of one, two, three or four types of read-capping nucleotide analogs can be linked to a PEG moiety having a different functional group including a methoxy group (OCH₃), amino group (NH₂), carboxyl group (COOH) and/or hydroxyl group (OH).

In some embodiments, a read-capping nucleotide analog comprises a sugar moiety, such as a cyclic moiety (see, e.g., Ferraro and Gotor 2000 Chem. Rev. 100: 4319-48, which is incorporated herein by reference for examples of cyclic moieties that may be present in the read-capping nucleotide analogs described herein), an acyclic moiety (see, e.g., Martinez, et al., 1999Nucleic Acids Research 27: 1271-1274; Martinez, et al., 1997 Bioorganic & Medicinal Chemistry Letters vol. 7: 3013-3016, each of which is incorporated herein by reference for examples of acyclic moieties that may be present in the read-capping nucleotide analogs described herein), or other sugar moieties (see, e.g., Joeng, et al., 1993 J. Med. Chem. 36:2627-2638; Kim, et al., 1993 J. Med. Chem. 36: 30-7; Eschenmosser 1999 Science 284: 2118-2124; and U.S. Pat. No. 5,558,991, each of which is incorporated herein by reference for examples of sugar moieties that may be present in the read-capping nucleotide analogs described herein). In some embodiments, a read-capping nucleotide analog comprises a sugar moiety comprising: ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, a read-capping nucleotide analog comprises a hydroxyl group at the 3′ sugar position. In some embodiments, the read-capping nucleotide analog comprises a chain terminating moiety at the 3′ sugar position. The chain terminating moiety may comprise H, F, NH₂, or an amino group.

In some embodiments, a read-capping nucleotide analog comprises a sugar moiety having a 3′OH group. In some embodiments, the read-capping nucleotide analogs comprise a chain terminating moiety at the 2′ or 3′ sugar position. In some embodiments, the chain terminating moiety is removable or cleavable from the 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, an alkenyl group, an alkynyl group, an allyl group, an aryl group, a benzyl group, an azide group, an amine group, an amide group, a keto group, an isocyanate group, a phosphate group, a thio group, a disulfide group, a carbonate group, a urea group, or a silyl group. In some embodiments, the chain terminating moiety is cleavable/or removable from the nucleotide, for example by reacting the chain terminating moiety with a chemical agent, by pH change, by light or by heat. For example, in some embodiments, chain terminating moieties comprising alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, chain terminating moieties comprising benzyl are cleavable with H2 Pd/C. In some embodiments, chain terminating moieties comprising amine, amide, keto, isocyanate, phosphate, thio, disulfide are cleavable with phosphine or with a thiol group including beta-mercaptoethanol or dithiothritol (DTT). In some embodiments, chain terminating moiety comprising carbonate is cleavable with potassium carbonate (K₂CO₃) in MeOH, with triethylamine in pyridine, or with Zn in acetic acid (AcOH). In some embodiments, chain terminating moieties comprising urea and silyl are cleavable with tetrabutylammonium fluoride, pyridine-HF, with ammonium fluoride, or with triethylamine trihydrofluoride. In some embodiments, a chain terminating moiety may be cleavable or removable with nitrous acid. In some embodiments, a chain terminating moiety may be cleavable or removable using a solution comprising nitrite, such as, for example, a combination of nitrite with an acid such as an organic acid, (e.g., acetic acid), sulfuric acid, or nitric acid.

In some embodiments, the 2′ or 3′ chain terminating moiety of the read-capping nucleotide analogs comprises an azide group, an azido group or an azidomethyl group. In some embodiments, the chain terminating moiety comprises a 3′-O-azido or 3′-O-azidomethyl group. In some embodiments, the chain terminating moieties comprising azide, azido and azidomethyl group are cleavable or removable with a cleaving agent such as a phosphine compound. In some embodiments, the phosphine compound comprises a derivatized tri-alkyl phosphine moiety. In some embodiments, the phosphine compound comprises a derivatized tri-aryl phosphine moiety. In some embodiments, the phosphine compound comprises Tris(2-carboxyethyl)phosphine (TCEP). In some embodiments, the phosphine compound comprises bis-sulfo triphenyl phosphine (BS-TPP). In some embodiments, the phosphine compound comprises Tri(hydroxyproyl)phosphine (THPP). In some embodiments, the cleaving agent comprises 4-dimethylaminopyridine (4-DMAP).

In some embodiments, the 2′ or 3′ chain terminating moiety of the read-capping nucleotide analogs comprises a 3′-O-amino group or a derivative thereof, which can be cleaved with nitrous acid through a mechanism utilizing nitrous acid, or using a solution comprising nitrous acid. In some embodiments, the 2′ or 3′ chain terminating moiety of the read-capping nucleotide analogs comprises a 3′-O-aminomethyl group, or a derivative thereof, which can be cleaved with nitrous acid through a mechanism utilizing nitrous acid, or using a solution comprising nitrous acid. In some embodiments, the 2′ or 3′ chain terminating moiety of the read-capping nucleotide analogs comprises a 3′-O-methylamino group or a derivative thereof, which can be cleaved with nitrous acid through a mechanism utilizing nitrous acid, or using a solution comprising nitrous acid.

In some embodiments, the chain terminating moiety comprises a 3′-O-amino group or a derivative thereof, which can be cleaved using a solution comprising nitrite. In some embodiments, the chain terminating moiety comprises a 3′-O-aminomethyl group or a derivative thereof, which can be cleaved using a solution comprising nitrite. In some embodiments, the chain terminating moiety a 3′-O-methylamino group, or a derivative thereof, which can be cleaved using a solution comprising nitrite. In some embodiments, for example, nitrite may be combined with or contacted with an acid such as acetic acid, sulfuric acid, or nitric acid. In some further embodiments, for example, nitrite may be combined with or contacted with an organic acid such as formic acid, acetic acid, propionic acid, butyric acid, isobutyric acid, or the like.

In some embodiments, a read-capping nucleotide analog comprises a 3′-deoxy nucleotide, 2′,3′-dideoxynucleotide, or a nucleotide that comprises one of the following 3′ modifications: 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, 3-O-benzyl, or derivatives thereof.

In some embodiments, the read-capping nucleotide analogs comprise a chain of 1, 2, 3, 4, 5, 6, 7, 8, 9, 10 or more phosphorus atoms where the chain is typically attached to the 5′ carbon of the sugar moiety via an ester or phosphoramide linkage. In some embodiments, the nucleotide is an analog having a phosphorus chain in which the phosphorus atoms are linked together with intervening O, S, NH, methylene or ethylene. In some embodiments, the phosphorus atoms in the chain comprises substituted side groups such as O, S or BH3. In some embodiments, the chain comprises phosphate analogs such as phosphoramidate, phosphorothioate, phosphorodithioate, and O-methylphosphoroamidite groups. In some embodiments, the chain is a polyphosphate chain. In some embodiments, the chain is a mix of phosphate and phosphoramidate groups. In some embodiments, the chain is a mix of phosphate and phosphorothioate groups. In some embodiments, the chain is a mix of phosphate, phosphoramidate, and phosphorothioate groups.

### Complexed Polymerases with Capped Sequencing Read Products

In another aspect, the present disclosure provides a composition comprising a plurality of complexed polymerases, wherein individual complexed polymerases comprise at least one polymerase complexed with a single-stranded nucleic acid template molecule having two or more positions along the single-stranded nucleic acid template molecule hybridized with a capped sequencing read product (e.g., a plurality of capped sequencing read products hybridized thereon). In some embodiments, each capped sequencing read product along the single-stranded nucleic acid template molecule is bound to a sequencing polymerase. In some embodiments, each capped sequencing read product comprises a sequencing primer (e.g., an oligonucleotide) joined to a polynucleotide having a sequence that is complementary to at least a portion of the single-stranded nucleic acid template molecule, and the terminal 3′ ends of individual capped sequencing read products comprise an incorporated read-capping nucleotide analog. In some embodiments, the polynucleotide can be synthesized via polymerase-catalyzed primer extension reaction in a template-dependent manner. In some embodiments, the plurality of single-strand nucleic acid template molecules can be immobilized to a support or can be immobilized to a coating on the support.

In some embodiments, individual single-stranded nucleic acid template molecules have hybridized along their lengths a plurality of capped sequencing read products. In some embodiments, each capped sequencing read product comprises a read-capping nucleotide analog incorporated at its terminal 3′ end, wherein the read-capping nucleotide analog can inhibit binding of a multivalent molecule to a complexed polymerase, or can substantially reduce the stability of binding between the multivalent molecule(s) and a complexed polymerase, so as to substantially reduce the persistence time. Thus, binding between complexed polymerases and detectably labeled multivalent molecules is not detectable or exhibits substantially reduced detectability. In some embodiments, a multivalent molecule comprises a core attached to multiple nucleotide arms, and individual nucleotide arms are attached to nucleotide units. In some embodiments, a multivalent molecule comprises (1) a core; and (2) a plurality of nucleotide arms, each arm comprising (i) a core attachment moiety, (ii) a spacer comprising a linker, and (iii) a nucleotide unit, wherein the core is attached to the plurality of nucleotide arms, wherein the spacer is attached to the linker, wherein the linker is attached to the nucleotide unit (e.g., see FIGS. 19-23).

In some embodiments, individual template molecules have hybridized along their lengths a plurality of capped sequencing read products. In some embodiments, each capped sequencing read product comprises a read-capping nucleotide analog incorporated at its terminal 3′ end, wherein the read-capping nucleotide analog can inhibit polymerase-catalyzed incorporation of a nucleotide or a chain terminating nucleotide, or can substantially reduce polymerase-catalyzed incorporation of a nucleotide or a chain terminating nucleotide. Thus, the read-capping nucleotide analogue prevents the complexed polymerases along the template molecule from carrying out primer extension.

In some embodiments, the capped sequencing read products comprise a sequencing primer (e.g., a nucleic acid oligonucleotide) which is joined to a polynucleotide having a sequence that is complementary to at least a portion of a single-stranded nucleic acid template molecule, and the terminal 3′ end of the capped sequencing read product comprises an incorporated read-capping nucleotide analog. The capped sequencing read product can be hybridized to a portion of the single-stranded template molecule. The capped sequencing read product can be synthesized via a polymerase-catalyzed primer extension reaction in a template-dependent manner. At least a portion of the complementary polynucleotide can be generated by any type of sequencing workflow comprising a polymerase-catalyzed primer extension reaction in which the sequence of the single-strand template molecule is determined. At least a portion of the complementary polynucleotide can be generated by any type of polymerase-catalyzed primer extension reaction in which the sequence of the single-strand template molecule is not determined.

In some embodiments, any portion of the single-stranded template molecule is immobilized to the support, or immobilized to a coating on the support. For example, the 5′ or 3′ end of the template molecule, or an internal portion of the template molecule, or a combination thereof, can be immobilized to the support. In some embodiments, the 5′ and/or 3′ end of the single-stranded nucleic acid template molecule is covalently attached to a surface primer which is immobilized to the support or immobilized to a coating on the support. In some embodiments, the 5′ and/or 3′ region of the single-stranded nucleic acid template molecule is hybridized to a surface primer which is immobilized to the support or immobilized to a coating on the support.

In some embodiments, the single-stranded nucleic acid template molecule can be generated by a clonal amplification workflow. In some embodiments, the single-stranded nucleic acid template molecule is not generated by a clonal amplification workflow.

In some embodiments, the single-stranded nucleic acid template molecule comprises at least one uridine nucleotide. In some embodiments, the single-stranded nucleic acid template molecule does not comprise a uridine nucleotide.

In some embodiments, the single-stranded nucleic acid template molecule comprises one copy of the sequence-of-interest. The one-copy template molecule can be generated by bridge amplification. In some embodiments, the single-stranded nucleic acid template molecule comprises two or more tandem copies of the sequence-of-interest. For example, the tandem-copy template molecule comprises a concatemer which can be generated by rolling circle amplification (RCA).

In some embodiments, the single-stranded nucleic acid template molecule comprises at least one sequence-of-interest (e.g., insert region). In some embodiments, the single-stranded nucleic acid template molecule further comprises at least one universal adaptor sequence. The universal adopter sequence may comprise a first surface primer binding site (e.g., capture primer binding site); a second surface primer binding site (e.g., surface pinning primer binding site); a first sequencing primer binding site (e.g., forward sequencing primer binding site, sometimes also referred to as a first universal sequencing primer binding site); a second sequencing primer binding site (e.g., reverse sequencing primer binding site, sometimes also referred to as a second universal sequencing primer binding site); a first amplification primer binding site (e.g., forward amplification primer binding site); a second amplification primer binding site (e.g., reverse amplification primer binding site); a first sample index sequence; a second sample index sequence; a first unique molecular tag sequence; a second unique molecular tag sequence; a first compaction oligonucleotide binding site; and/or a second compaction oligonucleotide binding site.

In some embodiments, the single-stranded nucleic acid template molecule comprises a first surface primer binding site. In some embodiments, the first surface primer binding site (or a complementary sequence thereof) can hybridize to at least a portion of the immobilized first surface primer. In some embodiments, the single-stranded nucleic acid template molecule comprises a second surface primer binding site. In some embodiments, the second surface primer binding site (or a complementary sequence thereof) can hybridize to at least a portion of the immobilized second surface primer. In some embodiments, the single-stranded nucleic acid template molecule comprises a first sequencing primer binding site. In some embodiments, the first sequencing primer site (or a complementary sequence thereof) can hybridize to at least a portion of the forward sequencing primer (sometimes referred to as a first universal sequencing primer). In some embodiments, the single-stranded nucleic acid template molecule comprises a second sequencing primer binding site. In some embodiments, the second sequencing primer site (or a complementary sequence thereof) can hybridize to at least a portion of the reverse sequencing primer (sometimes referred to as a second universal sequencing primer). In some embodiments, the single-stranded nucleic acid template molecule comprises a first amplification primer binding site. In some embodiments, the first amplification primer binding site (or a complementary sequence thereof) can hybridize to at least a portion of a forward amplification primer. In some embodiments, the single-stranded nucleic acid template molecule comprises a second amplification primer binding site. In some embodiments, the second amplification primer binding site (or a complementary sequence thereof) can hybridize to at least a portion of a reverse amplification primer. In some embodiments, the single-stranded nucleic acid template molecule comprises a first compaction oligonucleotide binding site. In some embodiments, the first compaction oligonucleotide binding site (or a complementary sequence thereof) can hybridize to at least a portion of a first compaction oligonucleotide. In some embodiments, the single-stranded nucleic acid template molecule comprises a second compaction oligonucleotide binding site. In some embodiments, the second compaction oligonucleotide binding site (or a complementary sequence thereof) can hybridize to at least a portion of a second compaction oligonucleotide.

In some embodiments, the support is a planar support (e.g., a slide). In some embodiments, the support is a non-planar support (e.g., a bead). In some embodiments, the support comprises or consists of a solid material. In some embodiments, the support comprises or consists of a semi-solid material. In some embodiments, the support comprises or consists of a porous material. In some embodiments, the support comprises or consists of a semi-porous material. In some embodiments, the support comprises or consists of a non-porous material. The support can be made of any suitable material known in the art or described herein, such as glass, plastic or a polymer material.

In some embodiments, the surface of the support can be coated with one or more compounds to produce a passivated layer on the support. In some embodiments, the passivated layer forms a porous layer. In some embodiments, the passivated layer forms a semi-porous layer. In some embodiments, the surface primer or the single-stranded nucleic template molecule can be attached to the passivated layer to immobilize the primer or template molecule to the support. In some embodiments, the support comprises a low non-specific binding surface that enables improved nucleic acid hybridization, amplification and sequencing performance on the support. In general, the support may comprise one or more layers of a covalently or non-covalently attached low-binding, chemical modification layers, e.g., silane layers, polymer films, and one or more covalently or non-covalently attached oligonucleotides that can be used for immobilizing a plurality of nucleic acid template molecules to the support. In some embodiments, the support can comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacet-amidylpentyl)acrylamide-co-acrylamide (PAZAM). In some embodiments, the support comprises a surface coating having at least one hydrophilic polymer coating layer and at least one layer of a plurality of oligonucleotides. The hydrophilic polymer coating layer can comprise polyethylene glycol (PEG). The hydrophilic polymer coating layer can comprise branched PEG having at least 4 branches. In some embodiments, the low non-specific binding coating has a degree of hydrophilicity which can be measured as a water contact angle, where the water contact angle is no more than 45 degrees.

In some embodiments, in the compositions comprising complexed polymerases, the support comprises a plurality of single-stranded nucleic acid template molecules immobilized to the support or immobilized to a coating on the support. In some embodiments, about 10²-10¹⁵ template molecule are immobilized to the support, with each template molecule being immobilized to a different site on the support. In some embodiments, the plurality of template molecule are immobilized to pre-determined sites (e.g., locations) on the support. In some embodiments, the plurality of template molecules are immobilized to random sites (e.g., locations) on the support. In some embodiments, the plurality of immobilized template molecules are in fluid communication with each other to permit flowing a solution of reagents (e.g., enzymes including polymerases, multivalent molecules, nucleotides and/or divalent cations, and the like) onto the support so that the plurality of immobilized template molecules on the support can be reacted with the solution of reagents in a massively parallel manner.

### Methods of Using Read-capping Nucleotide Analogs—Sequencing Template Strands

In one aspect, the present disclosure provides a method for sequencing two or more regions of a nucleic acid template molecule, comprising step (a): providing a plurality of nucleic acid template molecules, wherein individual template molecules comprise (i) a first region and a first universal sequencing primer binding site and (ii) a second region and a second universal sequencing primer binding site (as shown in, e.g., FIG. 3A or 3B). In some embodiments, individual template molecules in the plurality of nucleic acid template molecules comprise single-stranded nucleic acid template molecules. In some embodiments, individual template molecules in the plurality of nucleic acid template molecules comprise double-stranded nucleic acid template molecules. In some embodiments, the template molecules comprise at least one sequence of interest (e.g., an insert sequence) and at least one universal adaptor sequence. In some embodiments, the first regions in the template molecules of step (a) comprise a sequence of interest and/or a universal adaptor sequence. In some embodiments, the second regions in the template molecules of step (a) comprise a sequence of interest and/or a universal adaptor sequence. In some embodiments, individual template molecules comprise one copy of a sequence of interest and at least one universal adaptor sequence. In some embodiments, individual template molecules comprise nucleic acid concatemers comprising two or more tandem copies of a unit, wherein each unit comprises a sequence of interest and one or more universal adaptor sequences. In some embodiments, the first region and the second region comprise the same sequence of interest. In some embodiments, the first universal sequencing primer binding site and the second universal sequencing primer binding site have the same sequence. In some embodiments, the first region and the second region comprise the same sequence of interest and/or the same universal adaptor sequence.

In some embodiments, in step (a), individual template molecules comprise any one or any combination of two or more universal adaptor sequences. The universal adaptor sequences may comprise (i) a universal binding sequence for a forward sequencing primer, (ii) a universal binding sequence for a reverse sequencing primer, (iii) a universal binding sequence for an immobilized first surface primer, (iv) a universal binding sequence for an immobilized second surface primer, (v) a universal binding sequence for a first amplification primer, (vi) a universal binding sequence for a second amplification primer, (vii) a universal binding sequence for a compaction oligonucleotide and/or (viii) a sample barcode sequence. In some embodiments, individual template molecules comprise one or more unique molecular index (UMI) sequence(s).

In some embodiments, in step (a), the universal adaptor sequence(s) comprise a universal binding sequence for a forward sequencing primer. In some embodiments, the universal binding sequence (or a complementary sequence thereof) for the forward sequencing primer can hybridize to at least a portion of a forward sequencing primer. In some embodiments, the universal adaptor sequence(s) comprise a universal binding sequence for a reverse sequencing primer. In some embodiments, the universal binding sequence (or a complementary sequence thereof) for the reverse sequencing primer can hybridize to at least a portion of a reverse sequencing primer. In some embodiments, the universal adaptor sequence(s) comprise a universal binding sequence for an immobilized first surface primer. In some embodiments, the universal binding sequence (or a complementary sequence thereof) for the immobilized first surface primer can hybridize to at least a portion of an immobilized first surface primer. In some embodiments, the universal adaptor sequence(s) comprise a universal binding sequence for an immobilized second surface primer. In some embodiments, the universal binding sequence (or a complementary sequence thereof) for the immobilized second surface primer can hybridize to at least a portion of an immobilized second surface primer. In some embodiments, the universal adaptor sequence(s) comprise a universal binding sequence for a first amplification primer. In some embodiments, the universal binding sequence (or a complementary sequence thereof) for the first amplification primer can hybridize to at least a portion of a first amplification primer. In some embodiments, the universal adaptor sequence(s) comprise a universal binding sequence for a second amplification primer. In some embodiments, the universal binding sequence (or a complementary sequence thereof) for the second amplification primer can hybridize to at least a portion of a second amplification primer. In some embodiments, the universal adaptor sequence(s) comprise a universal binding sequence for a compaction oligonucleotide. In some embodiments, the universal binding sequence (or a complementary sequence thereof) for the compaction oligonucleotide can hybridize to at least a portion of a compaction oligonucleotide.

In some embodiments, the methods comprise step (b): providing a plurality of first nucleic acid sequencing primers and a plurality of second nucleic acid sequencing primers (as shown, e.g., in FIG. 3A or 3B). In some embodiments, the plurality of first nucleic acid sequencing primers comprise soluble oligonucleotides that hybridize to at least a portion of a universal binding sequence which is proximal or adjacent to the first region of the template molecule to be sequenced. In some embodiments, the first sequencing primers comprise 3′ OH extendible ends. In some embodiments, the first sequencing primers comprise a 3′ blocking moiety which can be removed to generate 3′ OH extendible ends. In some embodiments, the plurality of second nucleic acid sequencing primers comprise soluble oligonucleotides that hybridize to at least a portion of a universal binding sequence which is proximal or adjacent to the second region of the template molecule to be sequenced. In some embodiments, the second sequencing primers comprise 3′ OH extendible ends. In some embodiments, the second sequencing primers comprise a 3′ blocking moiety which can be removed to generate 3′ OH extendible ends.

In some embodiments, the methods comprise step (c): hybridizing individual first nucleic acid sequencing primers to the first universal sequencing primer binding sites on individual template molecules (shown in, e.g., FIG. 4A or 4B). In some embodiments, hybridizing one or more individual first sequencing primers to the first universal sequencing primer binding sites on individual template molecules generates one or more nucleic acid duplexes.

In some embodiments, the methods comprise step (d): sequencing the first regions on the plurality of nucleic acid template molecules thereby generating a plurality of first sequencing read products (see, e.g., FIG. 4A or 4B). In some embodiments, step (d) can be conducted using any type of sequencing workflow. In some embodiments, step (d) comprises contacting the nucleic acid duplexes from step (c) with a plurality of sequencing polymerases under conditions suitable for binding the sequencing polymerases to the nucleic acid duplexes and for generating a plurality of complexed polymerases. In some embodiments, step (d) comprises contacting the plurality of complexed polymerases with a plurality of nucleotide reagents. In some embodiments, the individual nucleotide reagents in the plurality comprise a canonical nucleotide, a chain terminating nucleotide or a multivalent molecule.

In some embodiments, step (d) comprises contacting the plurality of complexed polymerases with a plurality of detectably labeled chain terminating nucleotides under conditions suitable to incorporate a labeled chain terminating nucleotide into the terminal 3′ end of a hybridized first sequencing primer, and detecting and identifying the incorporated chain terminating nucleotide. In some embodiments, step (d) comprises conducting at least one polymerase-catalyzed nucleotide incorporation reaction on the plurality of template molecules to generate a plurality of first sequencing read products comprising a first sequencing primer joined to a polynucleotide having a sequence that is complementary to at least a portion of the nucleic acid template molecule. In some embodiments, step (d) comprises removing the chain terminating moiety and/or removing the detectable label from the incorporated chain terminating nucleotide to generate an extendible 3′OH sugar group on the chain terminating nucleotide. In some embodiments, step (d) comprises repeating at least once the steps of: (i) incorporating a detectably labeled chain terminating nucleotide into the terminal 3′ end of a hybridized first sequencing primer; (ii) detecting and identifying the chain terminating nucleotide; and (iii) removing the chain terminating moiety and/or removing the detectable label from the chain terminating nucleotide to generate an extendible 3′OH sugar group on the chain terminating nucleotide.

In some embodiments, step (d) comprises conducting a two-stage sequencing workflow. The first sequencing stage of the two-stage sequencing workflow comprises contacting the plurality of complexed polymerases with a plurality of detectably labeled multivalent molecules. In some embodiments, individual multivalent molecules comprise a core attached to multiple nucleotide arms, and the nucleotide arms are attached to nucleotide units. In some embodiments, the plurality of complexed polymerases are contacted with the plurality of multivalent molecules under conditions suitable to bind a nucleotide unit to a complexed polymerase and suitable to inhibit incorporation of the nucleotide unit into the terminal 3′ end of a hybridized first sequencing primer. The first sequencing stage of the two-stage sequencing work flow may further comprise detecting and identifying the bound nucleotide unit of the detectably labeled multivalent molecule. In some embodiments, the plurality of complexed polymerase and multivalent molecules are removed. In some embodiments, the second sequencing stage of the two-stage sequencing work flow comprises contacting the nucleic acid duplexes with a second plurality of sequencing polymerases under conditions suitable for binding the second sequencing polymerases to the nucleic acid duplexes to generate a second plurality of complexed polymerases. In some embodiments, the second sequencing stage of the two-stage sequencing work flow comprises contacting the second plurality of complexed polymerases with a plurality of non-labeled nucleotide analogs under conditions suitable to incorporate a nucleotide analog into the terminal 3′ end of a hybridized first sequencing primer. In some embodiments, step (d) comprises conducting one or more cycles of the two-stage sequencing workflow on the plurality of template molecule to generate a first plurality of sequencing read products, the read products comprising a first sequencing primer joined to a polynucleotide having a sequence that is complementary to at least a portion of the nucleic acid template molecule.

In some embodiments, the methods for sequencing two or more regions of a nucleic acid template molecule comprise step (e): conducting a plurality of first capping reactions, the capping reactions comprising incorporating a read-capping nucleotide analog into the terminal 3′ end of individual first sequencing read products, thereby generating a plurality of capped first sequencing read products (shown in, e.g., FIG. 5A or 5B). In some embodiments, the plurality of first capping reactions comprise one or more polymerase-catalyzed nucleotide incorporation reactions. In some embodiments, the plurality of first capping reactions comprises contacting the plurality of first sequencing read products with a plurality of polymerases and a plurality of read-capping nucleotide analogs under conditions suitable for incorporating a read-capping nucleotide analog into the terminal 3′ end of individual first sequencing read products, thereby generating a plurality of capped first sequencing read products. In some embodiments, the polymerase comprises a sequencing polymerase that can catalyze incorporation of a nucleotide analog having a base-linked terminator moiety. In some embodiments, one or more read-capping nucleotide analogs are incorporated into the terminal 3′ end of individual first sequencing read products. In some embodiments, incorporation of a read-capping nucleotide analog can be conducted at a temperature of about 25-50 °C, or about 50-55 °C. In some embodiments, incorporation of a read-capping nucleotide analog can be conducted for about 2-20 minutes.

In some embodiments, in step (e), the read-capping nucleotide analogs comprises (i) a heterocyclic base, (ii) a sugar, and (iii) a polyphosphate chain, wherein the heterocyclic base is linked to a terminating moiety that blocks binding of a multivalent molecule to the terminal 3′ ends of the first capped sequencing read products. In some embodiments, the read-capping nucleotide analogs further comprise a sugar moiety with an extendible 3′OH group. In some embodiments, the read-capping nucleotide analogs further comprise a sugar moiety with a non-extendible chain terminating group at the 3′ position. Exemplary read-capping nucleotide analogs are shown in FIGS. 8A-8J and 9 and are described in more detail below. In some embodiments, the sugar comprises 5 carbons, i.e. a pentose sugar. In some embodiments, the sugar comprises a ribose or deoxyribose.

In some embodiments, in step (e), the read-capping nucleotide analogs comprise a heterocyclic base which is linked to a terminating moiety that blocks binding of a multivalent molecule to the terminal 3′ ends of the first capped sequencing read products. In some embodiments, individual multivalent molecules comprise a core attached to multiple nucleotide arms, and individual nucleotide arms are attached to nucleotide units (see e.g., FIGS. 19-23). In some embodiments, the base-linked terminating moiety also blocks polymerase-catalyzed incorporation of a subsequent nucleotide (e.g., a canonical nucleotide or a chain terminating nucleotide) into the capped sequencing read product.

In some embodiments, the methods comprise step (f): retaining the plurality of capped first sequencing read products which are hybridized to their respective nucleic acid template molecules (shown in, e.g., FIGS. 5A, 5B, 6A, 6B, 7A and 7B). In some embodiments, the terminating moiety incorporated at the terminal 3′ end of individual first capped sequencing read products is not cleaved or removed. In some embodiments, the retained plurality of capped first sequencing read products are not denatured from their respective nucleic acid template molecules with heat and/or with a chemical denaturation reagent. In some embodiments, the retained plurality of capped first sequencing read products are not degraded with an enzyme having a 5′ to 3′ exonuclease activity (e.g., T7 exonuclease or lambda exonuclease). In some embodiments, the retained plurality of capped first sequencing read products are not degraded with an enzyme having a 3′ to 5′ exonuclease activity (e.g., E. coli exonuclease I or E. coli exonuclease III).

In some embodiments, the methods comprise step (g): hybridizing a plurality of second sequencing primers to a plurality of second universal sequencing primer binding sites, wherein the second universal primer binding sites are present on individual nucleic acid template molecules (see e.g., FIG. 6A or 6B). In some embodiments, hybridizing the plurality of second sequencing primers to the plurality of second universal sequencing primer binding sites generates one or more nucleic acid duplexes. In some embodiments, the first and second sequencing primers hybridize to different and non-overlapping regions of the same nucleic acid template molecule. In some embodiments, the first and second sequencing primers hybridize to overlapping regions of the same nucleic acid template molecule. In some embodiments, the first and second sequencing primers hybridize to the same region of the same nucleic acid template molecule. In some embodiments, step (g) comprises hybridizing individual second nucleic acid sequencing primers to the second universal sequencing primer binding sites on the individual template molecules.

In some embodiments, the methods comprise step (h): sequencing the second regions on the plurality of nucleic acid template molecules, thereby generating a plurality of second sequencing read products (shown in, e.g., FIG. 6A or 6B). In some embodiments, the sequencing of step (h) comprises contacting individual nucleic acid duplexes from step (g) with a plurality of sequencing polymerases under conditions suitable for binding the sequencing polymerases to the nucleic acid duplex to generate a plurality of complexed polymerases. In some embodiments, the sequencing comprises contacting the plurality of complexed polymerases with a plurality of nucleotide reagents. In some embodiments, the nucleotide reagents comprise a canonical nucleotide, a chain terminating nucleotide and/or a multivalent molecule.

In some embodiments, step (h) comprises contacting the plurality of complexed polymerases with a plurality of detectably labeled chain terminating nucleotides under conditions suitable to incorporate a labeled chain terminating nucleotide into the terminal 3′ end of a hybridized second sequencing primers, and detecting and identifying the chain terminating nucleotides. In some embodiments, the sequencing of step (h) comprises conducting at least one polymerase-catalyzed nucleotide incorporation reaction on the plurality of template molecules to generate a plurality of second sequencing read products, wherein individual read products in the plurality of second sequencing read products comprise a second sequencing primer joined to a polynucleotide having a sequence that is complementary to at least a portion of the nucleic acid template molecule.

In some embodiments, step (h) comprises conducting a two-stage sequencing workflow. The first sequencing stage of the two-stage sequencing work flow may comprise contacting the complexed polymerases with a plurality of detectably labeled multivalent molecules. In some embodiments, individual multivalent molecules comprise a core attached to multiple nucleotide arms and individual nucleotide arms are attached to a nucleotide unit. In some embodiments, the complexed polymerases are contacted with the multivalent molecules under conditions suitable to bind a nucleotide unit to a complexed polymerase and suitable to inhibit incorporation of the nucleotide unit into the terminal 3′ end of a hybridized second sequencing primer. The first sequencing stage of the two-stage sequencing work flow may further comprise detecting and identifying the bound nucleotide units of the detectably labeled multivalent molecules. In some embodiments, the complexed polymerases and multivalent molecules are removed. In some embodiments, the second sequencing stage of the two-stage sequencing work flow comprises contacting the plurality of nucleic acid duplexes with a second plurality sequencing polymerase under conditions suitable for binding the second sequencing polymerases to the nucleic acid duplexes to generate a second plurality of complexed polymerases. In some embodiments, the second sequencing stage of the two-stage sequencing work flow further comprises contacting the second plurality of complexed polymerases with a plurality of non-labeled nucleotide analogs under conditions suitable to incorporate a nucleotide analog into the terminal 3′ end of a hybridized second sequencing primers. In some embodiments, the sequencing of step (h) comprises conducting one or more cycles of the two-stage sequencing workflow on the plurality of template molecules to generate a plurality of second sequencing read products, wherein individual second sequencing read produces comprise a second sequencing primer joined to a polynucleotide having a sequence that is complementary to at least a portion of the nucleic acid template molecule.

In some embodiments, the methods comprise step (i): conducting a plurality of second capping reactions comprising incorporating a read-capping nucleotide analog into the terminal 3′ end of individual second sequencing read products in the plurality of second sequencing read products, thereby generating a second plurality of capped sequencing read products (shown in e.g., FIG. 7A or 7B). In some embodiments, the plurality of second capping reactions comprise one or more polymerase-catalyzed nucleotide incorporation reactions. In some embodiments, the plurality of second capping reactions comprises contacting the plurality of second sequencing read products with a plurality of polymerases and a plurality of read-capping nucleotide analogs under conditions suitable for incorporating a read-capping nucleotide analog into the terminal 3′ end of individual second sequencing read products, thereby generating a second plurality of capped sequencing read products. In some embodiments, the polymerase comprises a sequencing polymerase that can catalyze incorporation of a nucleotide analog having a base-linked terminator moiety. In some embodiments, one or more read-capping nucleotide analogs are incorporated into the terminal 3′ end of individual second sequencing read products in the plurality of second sequencing read products. In some embodiments, incorporation of a read-capping nucleotide analog can be conducted at a temperature of about 25-50 °C, or about 50-55 °C. In some embodiments, incorporation of a read-capping nucleotide analog can be conducted for about 2-20 minutes.

In some embodiments, in step (i), individual read-capping nucleotide analogs comprise (i) a heterocyclic base, (ii) a sugar, and (iii) a polyphosphate chain, wherein the heterocyclic base is linked to a terminating moiety that blocks binding of a multivalent molecule to the terminal 3′ ends of the second capped sequencing read products. In some embodiments, individual read-capping nucleotide analogs comprise a sugar moiety with an extendible 3′OH group. In some embodiments, individual read-capping nucleotide analogs comprise a sugar moiety with a non-extendible chain terminating group at the 3′ position. In some embodiments, the sugar comprises 5 carbons, i.e. a pentose sugar. In some embodiments, the sugar comprises a ribose or deoxyribose. Illustrative read-capping nucleotide analogs are shown in FIGS. 8A-8J and 9, and are described in more detail herein.

In some embodiments, in step (i), individual read-capping nucleotide analogs comprise a heterocyclic base which is linked to a terminating moiety that blocks binding of a multivalent molecule to the terminal 3′ ends of the sequencing read products in the plurality of capped second sequencing read products. In some embodiments, individual multivalent molecules comprise a core attached to multiple nucleotide arms, wherein individual nucleotide arms are attached to a nucleotide unit. In some embodiments, the base-linked terminating moiety also blocks polymerase-catalyzed incorporation of a subsequent nucleotide (e.g., a canonical nucleotide or a chain terminating nucleotide) into the capped sequencing read product.

In some embodiments, in step (i), individual sequenced template molecules comprise at least one capped first sequencing read product hybridized thereon and at least one capped second sequencing read product hybridized thereon.

In some embodiments, the methods for sequencing two or more regions of a nucleic acid template molecule comprise step (j1): removing the plurality of capped second sequencing read products which are hybridized to their respective template molecules. In some embodiments, the plurality of capped second sequencing read products can be denatured from their respective template molecules with heat and/or with a chemical denaturation reagent. In some embodiments, the plurality of capped second sequencing read products can be degraded with an enzyme having a 5′ to 3′ exonuclease activity (e.g., T7 exonuclease or lambda exonuclease). In some embodiments, the plurality of capped second sequencing read products can be degraded with an enzyme having a 3′ to 5′ exonuclease activity (e.g., E. coli exonuclease I or E. coli exonuclease III).

In some embodiments, the methods for sequencing two or more regions of a nucleic acid template molecule comprise step (j2): retaining the plurality of capped second sequencing read products which are hybridized to their respective template molecules, thereby producing a retained plurality of capped second sequencing read products. In some embodiments, the terminating moiety of the read-capping nucleotide analog that is incorporated at the terminal 3′ end of individual capped second sequencing read products is not cleaved or removed. In some embodiments, the retained plurality of capped second sequencing read products are not denatured from their respective template molecules with heat and/or with a chemical denaturation reagent. In some embodiments, the retained plurality of capped second sequencing read products are not degraded with an enzyme having a 5′ to 3′ exonuclease activity (e.g., T7 exonuclease or lambda exonuclease). In some embodiments, the retained plurality of capped second sequencing read products are not degraded with an enzyme having a 3′ to 5′ exonuclease activity (e.g., E. coli exonuclease I or E. coli exonuclease III).

In some embodiments, the methods comprise repeating steps (b)-(j1) or repeating steps (b)-(j2) using a third and, optionally, a fourth sequencing primer, wherein the third and fourth sequencing primers hybridize to a third and fourth region of the same template molecule, respectively. The skilled artisan recognizes that steps (b)-(j1) or steps (b)-(j2) can be repeated multiple times using a third, fourth, fifth, sixth, seventh, eighth, nineth, tenth or more sequencing primers that hybridize to their respective regions of the same template molecule.

In some embodiments, the third and the fourth sequencing primers hybridize to different and non-overlapping regions of the same nucleic acid template molecule. In some embodiments, the third and the fourth sequencing primers hybridize to different and non-overlapping regions of the same nucleic acid template molecule compared to the first and second sequencing primers. In some embodiments, the third and the fourth sequencing primers hybridize to overlapping regions of the same nucleic acid template molecule. In some embodiments, the third and the fourth sequencing primers hybridize to regions that overlap the binding sites of the first and second sequencing primers on the same nucleic acid template molecule. In some embodiments, the third and the fourth sequencing primers hybridize to the same region of the same nucleic acid template molecule. In some embodiments, the same region is the same region that the first and second sequencing primers hybridize to, of the same nucleic acid template molecule.

In some embodiments, any suitable portion of the single-stranded template molecule may be immobilized to the support or immobilized to a coating on the support. For example, the 5′ or 3′ end of the template molecule, or an internal portion of the template molecule, can be immobilized to the support. In some embodiments, the 5′ or 3′ end of the single-stranded nucleic acid template molecule is covalently attached to a surface primer which is immobilized to the support or immobilized to a coating on the support. In some embodiments, the 5′ or 3′ region of the single-stranded nucleic acid template molecule is hybridized to a surface primer which is immobilized to the support or immobilized to a coating on the support.

In some embodiments, the single-stranded nucleic acid template molecule can be generated by a clonal amplification workflow, for example rolling circle amplification or bridge amplification. In some embodiments, the single-stranded nucleic acid template molecule was not generated by a clonal amplification workflow.

In some embodiments, the single-stranded nucleic acid template molecule comprises at least one uridine nucleotide. In some embodiments, the single-stranded nucleic acid template molecule does not comprise a uridine nucleotide.

In some embodiments, the single-stranded nucleic acid template molecule comprises one copy of the sequence-of-interest. For example, the one-copy template molecule can be generated by bridge amplification. In some embodiments, the single-stranded nucleic acid template molecule comprises two or more tandem copies of the sequence-of-interest. For example, the tandem-copy template molecule comprises a concatemer which can be generated by rolling circle amplification (RCA).

In some embodiments, the support is a support (e.g., a slide). In some embodiments, the support is a non-planar support (e.g., a bead). In some embodiments, the support comprises or consists of a solid material. In some embodiments, the support comprises or consists of a semi-solid material. In some embodiments, the support comprises or consists of a porous material. In some embodiments, the support comprises or consists of a semi-porous material. In some embodiments, the support comprises or consists of a non-porous material. The support can be made of any suitable material known in the art or described herein, such as glass, plastic or a polymer material.

In some embodiments, the surface of the support can be coated with one or more compounds to produce a passivated layer on the support. In some embodiments, the passivated layer forms a porous layer. In some embodiments, the passivated layer forms a semi-porous layer. In some embodiments, the surface primer or the single-stranded nucleic template molecule can be attached to the passivated layer to immobilize the primer or template molecule to the support. In some embodiments, the support comprises a low non-specific binding surface that enables improved nucleic acid hybridization, amplification and sequencing performance on the support. In general, the support may comprise one or more layers of a covalently or non-covalently attached low-binding, chemical modification layers, e.g., silane layers, polymer films, and one or more covalently or non-covalently attached oligonucleotides that can be used for immobilizing a plurality of nucleic acid template molecules to the support. In some embodiments, the support can comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacet-amidylpentyl)acrylamide-co-acrylamide (PAZAM). In some embodiments, the support comprises a surface coating having at least one hydrophilic polymer coating layer and at least one layer of a plurality of oligonucleotides. The hydrophilic polymer coating layer can comprise polyethylene glycol (PEG). The hydrophilic polymer coating layer can comprise branched PEG having at least 4 branches. In some embodiments, the low non-specific binding coating has a degree of hydrophilicity which can be measured as a water contact angle, where the water contact angle is no more than 45 degrees.

In some embodiments, the support comprises a plurality of single-stranded nucleic acid template molecules immobilized to the support or immobilized to a coating on the support. In some embodiments, about 10²-10¹⁵ template molecule are immobilized to the support with each template molecule being immobilized to a different site on the support. In some embodiments, the plurality of template molecule are immobilized to pre-determined sites (e.g., locations) on the support. In some embodiments, the plurality of template molecules are immobilized to random sites (e.g., locations) on the support. In some embodiments, the plurality of immobilized template molecules are in fluid communication with each other to permit flowing a solution of reagents (e.g., enzymes including polymerases, multivalent molecules, nucleotides and/or divalent cations, and the like) onto the support so that the plurality of immobilized template molecules on the support can be reacted with the solution of reagents in a massively parallel manner.

### Methods for Pairwise Sequencing

In another aspect, the present disclosure provides methods for pairwise sequencing which comprise obtaining a first sequencing read of a first region of a first nucleic acid strand (e.g., sense strand or forward strand), and obtaining a second sequencing read of a second region of a second nucleic acid strand that is complementary to the first stand (e.g., anti-sense strand or reverse strand), wherein the first and second strands correspond to two complementary strands of a double stranded template molecule. The first sequencing read of the first sequenced region and the second sequencing read of the second sequenced region can comprise overlapping sequences which correspond to complementary sequences present on the first and second strands of the double stranded template molecule, respectively. The first and the second sequencing reads (sometimes referred to as paired sequencing reads) can be aligned so that the overlapping region of the paired sequencing reads can yield sequence information of a paired region in the original double stranded nucleic acid source (e.g., a paired region in the genome), and the accuracy of the sequence information can be ascertained from the first and second sequencing reads with a high level of confidence. However, the skilled artisan will appreciate that the first sequencing read of the first sequenced region and the second sequencing read of the second sequenced region do not necessarily need to have overlapping sequences. The first and the second sequencing reads can initiate at one end of their respective template molecules, or can initiate at an internal position.

The present disclosure provides pairwise sequencing methods, comprising step (a): providing a plurality of single stranded nucleic acid template molecules, wherein individual nucleic acid template molecules comprise at least one nucleotide having a scissile moiety. In some embodiments, individual nucleic acid template molecules in the plurality are immobilized to a first surface primer that is immobilized to a support. In some embodiments, the first surface primer lacks a nucleotide having a scissile moiety. In some embodiments, a plurality of second surface primers are immobilized to the support. In some embodiments, no second surface primers are immobilized to the support. In some embodiments, a plurality of first surface primers and a plurality of second surface primers are immobilized to the support. In some embodiments, the template molecules are immobilized.

In some embodiments, individual nucleic acid template molecules are immobilized to the support. In some embodiments, individual nucleic acid template molecules comprise concatemer molecules comprising multiple tandem copies of a sequence-of-interest and at least one universal adaptor sequence (e.g., a sequencing primer binding site). In some embodiments, individual nucleic acid template molecules are non-concatemer molecules comprising only one copy of the sequence-of-interest and at least one universal adaptor sequence (e.g., a sequencing primer binding site).

In some embodiments, individual nucleic acid template molecules are covalently joined to an immobilized surface primer (e.g., an immobilized first surface primer) (shown in e.g., FIG. 16A). In some embodiments, individual nucleic acid template molecules are hybridized to an immobilized surface primer (e.g., an immobilized first surface primer) (see e.g., FIG. 17).

In some embodiments, individual nucleic acid template molecules in the plurality of nucleic acid template molecules comprise at least one sequence-of-interest and at least one universal adaptor sequence or any combination of two or more of universal adaptor sequences. The universal adaptor sequence or combination of two or more universal adaptor sequences may comprise a first surface primer binding site (or a complementary sequence thereof) which can hybridize to any suitable portion of the immobilized first surface primer; a second surface primer binding site (or a complementary sequence thereof) which can hybridize to any suitable portion of the immobilized second surface primer; a first sequencing primer site (or a complementary sequence thereof) which can hybridize to any suitable portion of the forward sequencing primer; a second sequencing primer site (or a complementary sequence thereof) which can hybridize to any suitable portion of the reverse sequencing primer; a first amplification primer binding site (or a complementary sequence thereof) which can hybridize to any suitable portion of a forward amplification primer; a second amplification primer binding site (or a complementary sequence thereof) which can hybridize to any suitable portion of a reverse amplification primer; a first compaction oligonucleotide binding site (or a complementary sequence thereof) which can hybridize to any suitable portion of a first compaction oligonucleotide; a second compaction oligonucleotide binding site (or a complementary sequence thereof) which can hybridize to any suitable portion of a second compaction oligonucleotide; a first sample index sequence; a second sample index sequence; a first unique molecular tag sequence; and/or a second unique molecular tag sequence.

In some embodiments, the scissile moiety in the nucleic acid template molecules of step (a) can be converted into an abasic site in the nucleic acid template molecules. In some embodiments, the scissile moiety comprises uridine, 8-oxo-7,8-dihydroguanine (e.g., 8oxoG) or deoxyinosine. The uridine can be converted to an abasic site using uracil DNA glycosylase (UDG), the 8oxoG can be converted to an abasic site using FPG glycosylase (also referred to as DNA-formamidopyrimidine glycosylase), and the deoxyinosine can be converted to an abasic site using AlkA glycosylase (also referred to as 3-methyladenine-DNA glycosylase II). In some embodiments, individual nucleic acid template molecules include 1-20, 20-40, 40-60, 60-80, 80-100, or a higher number of nucleotides with a scissile moiety. In some embodiments, about 0.1-1%, or about 1-5%, or about 5-10%, or about 10-20%, or about 20-30% or a higher percent of the dTTP in individual nucleic acid template molecules are replaced with nucleotides having a scissile moiety. In some embodiments, the nucleotides having a scissile moiety are distributed at random positions along individual nucleic acid template molecules. In some embodiments, the nucleotides having a scissile moiety are distributed at different positions in the different nucleic acid template molecules.

In some embodiments, the first surface primers are immobilized to the support (“immobilized first surface primers,” also referred to as “immobilized first surface capture primers”). In some embodiments, the immobilized first surface primers comprise single stranded oligonucleotides comprising DNA. In some embodiments, the immobilized first surface primers comprises single stranded oligonucleotides comprising RNA. In some embodiments, the immobilized first surface primers comprises single stranded oligonucleotides comprising a combination of RNA and DNA. An immobilized first surface primer can be immobilized to the support or immobilized to a coating on the support. An immobilized first surface primer can be embedded and attached (coupled) to the coating on the support. In some embodiments, the 5′ end of the immobilized first surface primer is immobilized to a support or immobilized to a coating on the support. In some embodiments, an interior portion of the immobilized first surface primer is immobilized to a support or immobilized to a coating on the support. In some embodiments, the 3′ end of the immobilized first surface primer is immobilized to a support or immobilized to a coating on the support. In some embodiments, the support comprises a plurality of immobilized first surface primers having the same sequence. The immobilized first surface primers in the plurality of first surface primers can be any length, for example 4-50 nucleotides, or 50-100 nucleotides, or 100-150 nucleotides, or longer. In some embodiments, the 3′ terminal end of an immobilized first surface primer comprise an extendible 3′ OH moiety. In some embodiments, the 3′ terminal end of an immobilized first surface primer comprise a 3′ non-extendible moiety. In some embodiments, an immobilized first surface primer lacks a nucleotide having a scissile moiety.

In some embodiments, individual first surface primers in the plurality of immobilized first surface primers comprise at least one phosphorothioate diester bond at the 5′ end, which can render the first surface primer resistant to exonuclease degradation. In some embodiments, individual first surface primers in the plurality of immobilized first surface primers comprise 2, 3, 4, 5 or more consecutive phosphorothioate diester bonds at the 5′ ends. In some embodiments, individual first surface primers in the plurality of immobilized first surface primers comprise at least one ribonucleotide and/or at least one 2′-O-methyl or 2′-O-methoxyethyl (MOE) nucleotide which can render the first surface primer resistant to exonuclease degradation.

In some embodiments, individual first surface primers in the plurality of immobilized first surface primers comprise at least one locked nucleic acid (LNA) which comprises a methylene bridge bond between a 2′ oxygen and 4′ carbon of the pentose ring. An immobilized first surface primer comprising at least one LNA can be resistant to nuclease digestions and can exhibit increased melting temperature when hybridized to the forward extension strand.

In some embodiments, the nucleic acid template molecules comprise two or more copies of a universal binding sequence (or complementary sequence thereof) for an second surface primer immobilized to a support (“immobilized second surface primer”) having a sequence that differs from the first immobilized surface primer. The immobilized second surface primers of step (a) may comprise single stranded oligonucleotides comprising DNA, RNA or a combination of DNA and RNA. An immobilized second surface primer can be immobilized to the support or immobilized to a coating on the support. An immobilized second surface primer can be embedded and attached (coupled) to the coating on the support. In some embodiments, the 5′ end of an immobilized second surface primer is immobilized to a support or immobilized to a coating on the support. In some embodiments, an interior portion of an immobilized second surface primer is immobilized to a support or immobilized to a coating on the support. In some embodiments, the 3′ end of an immobilized second surface primer is immobilized to a support or immobilized to a coating on the support. In some embodiments, the support comprises a plurality of immobilized second surface primers having the same sequence. The immobilized second surface primers can be any length, for example 4-50 nucleotides, or 50-100 nucleotides, or 100-150 nucleotides, or longer.

In some embodiments, the 3′ terminal ends of individual second surface primer in the plurality of immobilized second surface primers comprise an extendible 3′ OH moiety. In some embodiments, the 3′ terminal end of an immobilized second surface primer comprises a 3′ non-extendible moiety. In some embodiments, the 3′ terminal end of an immobilized second surface primer comprises a moiety that blocks primer extension (e.g., non-extendible terminal 3′ end), such as for example a phosphate group, a dideoxycytidine group, an inverted dT, or an amino group. In some embodiments, an immobilized second surface primer is not extendible in a primer extension reaction. In some embodiments, an immobilized second surface primer lacks a nucleotide having a scissile moiety.

In some embodiments, in the methods for the pairwise sequencing, individual second surface primers in the plurality of immobilized second surface primers comprise at least one phosphorothioate diester bond at the 5′ ends which can render the second surface primer resistant to exonuclease degradation. In some embodiments, individual second surface primers in the plurality of immobilized second surface primers comprises 2, 3, 4, 5 or more consecutive phosphorothioate diester bonds at the 5′ ends. In some embodiments, individual second surface primers in the plurality of immobilized second surface primers comprise at least one ribonucleotide and/or at least one 2′-O-methyl or 2′-O-methoxyethyl (MOE) nucleotide which can render the second surface primer resistant to exonuclease degradation.

In some embodiments, individual single stranded nucleic acid template molecules are joined or immobilized to an immobilized first surface primer, and at least one portion of the individual template molecule is hybridized to an immobilized second surface primer. In such embodiments, the immobilized second surface primers serve to pin down a portion of the immobilized template molecules to the support (e.g., see FIG. 16H).

In some embodiments, the support comprises about 10²-10¹⁵ immobilized first surface primers per mm². In some embodiments, the support comprises about 10²-10¹⁵ immobilized second surface primers per mm². In some embodiments, the support comprises about 10²-10¹⁵ immobilized first surface primers and about 10²-10¹⁵ immobilized second surface primers per mm².

In some embodiments, the immobilized surface primers (e.g., first and second surface primers) are in fluid communication with each other to permit flowing various solutions of linear or circular nucleic acid template molecules, soluble primers, enzymes, nucleotides, divalent cations, buffers, reagents, and the like, onto the support so that the plurality of immobilized surface primers (and the primer extension products generated from the immobilized surface primers) react with the solutions in a massively parallel manner.

In some embodiments, the pairwise sequencing method comprises step (b): sequencing a first region of the plurality of nucleic acid template molecules, thereby generating a first plurality of forward extension duplexes. The sequencing of the first region of step (b) may comprise contacting the plurality of nucleic acid template molecules with a first plurality of forward sequencing primers under conditions suitable to hybridize at least one of the first forward sequencing primers to at least one of the forward sequencing primer binding sites located on the nucleic acid template molecules, and conducting a first set of forward sequencing reactions using one or more types of sequencing polymerases, and a plurality of nucleotide reagents (e.g., nucleotides and/or multivalent molecules). In some embodiments, the nucleic acid template molecules are immobilized. In some embodiments, the first plurality of forward sequencing primers are soluble (i.e., in solution). In some embodiments, the first plurality of forward sequencing primers are soluble and can hybridize to at least a region of a universal binding sequence which is proximal or adjacent to the first portion of the nucleic acid template molecules to be sequenced. The first forward sequencing reactions can generate a plurality of first forward extension duplexes comprising a first forward sequencing read product hybridized to a nucleic acid template molecule. Individual first forward sequencing read products may comprise a first forward sequencing primer joined to a polynucleotide having a sequence that is complementary to the first region of the nucleic acid template molecule to be sequenced. The forward sequencing primer binding site can be hybridized to a forward sequencing primer and can undergo a sequencing reaction. In some embodiments, a nucleic acid template molecule in the plurality of nucleic acid template molecules is an concatemer template molecule. In some embodiments, individual concatemer template molecules comprise multiple copies of the forward sequencing primer binding sites, wherein each forward sequencing primer binding site is capable of hybridizing to a first forward sequencing primer. Individual concatemer template molecule can undergo two or more sequence reactions simultaneously, wherein each sequencing reaction is initiated from a different first forward sequencing primer and each forward sequencing primer is hybridized to a different forward sequencing primer binding site (e.g., see FIG. 16B) on the individual concatemer template molecule. In some embodiments, a first forward sequencing primer comprises a 3′ OH extendible end. In some embodiments, a first forward sequencing primer comprises a 3′ blocking moiety which can be removed to generate a 3′ OH extendible end. In some embodiments, a first forward sequencing primer lacks a nucleotide having a scissile moiety. In some embodiments, the plurality of nucleotide reagents used in the sequencing reaction comprises a plurality of nucleotides (or analogs thereof) labeled with a detectable reporter moiety. In some embodiments, the plurality of nucleotide reagents comprises a plurality of multivalent molecules each having a core attached to multiple nucleotide arms, and individual nucleotide arms are attached to a nucleotide unit. In some embodiments the multivalent molecules are labeled with a detectable reporter moiety. In some embodiments, the core is labeled with a detectable reporter moiety. In some embodiments, at least one nucleotide arm and/or at least one nucleotide unit is labeled with a detectable reporter moiety. In some embodiments, the detectable reporter moiety comprises a fluorophore. An illustrative nucleotide arm is shown in FIG. 23, and illustrative multivalent molecules are shown in FIGS. 19-22.

In some embodiments, step (b) comprises: conducting a plurality of first capping reactions comprising incorporating a read-capping nucleotide analog into the terminal 3′ end of individual first sequencing read products, thereby generating a plurality of capped first sequencing read products. In some embodiments, the first capping reaction comprises a polymerase-catalyzed nucleotide incorporation reaction. In some embodiments, the plurality of first capping reactions comprises contacting the plurality of first sequencing read products with a plurality of polymerases and a plurality of read-capping nucleotide analogs under a condition suitable for incorporating a read-capping nucleotide analog into the terminal 3′ end of individual first sequencing read products, thereby generating a first plurality of capped sequencing read products. In some embodiments, the plurality of polymerases comprises sequencing polymerases that can catalyze incorporation of a nucleotide analog having a base-linked terminator moiety. In some embodiments, one or more read-capping nucleotide analogs are incorporated into the terminal 3′ end of individual first sequencing read products.

In some embodiments, in step (b), the read-capping nucleotide analog comprises (i) a heterocyclic base, (ii) a sugar, and (iii) a polyphosphate chain, wherein the heterocyclic base is linked to a terminating moiety that blocks binding of a multivalent molecule to the terminal 3′ end of a first capped sequencing read product. In some embodiments, the read-capping nucleotide analog further comprises a sugar moiety with an extendible 3′OH group. In some embodiments, the read-capping nucleotide analog further comprises a sugar moiety with a non-extendible chain terminating group in the 3′ position. In some embodiments, the sugar comprises 5 carbons, i.e. a pentose sugar. In some embodiments, the sugar comprises a ribose or deoxyribose. Illustrative read-capping nucleotide analogs are shown in FIGS. 8A-8J and 9, and are described in more detail below.

In some embodiments, in step (b), the read-capping nucleotide analog comprises a heterocyclic base which is linked to a terminating moiety that blocks binding of a multivalent molecule (see e.g., FIGS. 19-23) to the terminal 3′ ends of the capped first sequencing read products. In some embodiments, individual multivalent molecules comprise a core attached to multiple nucleotide arms and individual nucleotide arms are attached to a nucleotide unit. In some embodiments, the base-linked terminating moiety also blocks polymerase-catalyzed incorporation of a subsequent nucleotide (e.g., a canonical nucleotide or a chain terminating nucleotide) into the capped sequencing read product.

In some embodiments, step (b) comprises: retaining the plurality of capped first sequencing read products which are hybridized to the template molecules. In some embodiments, the terminating moiety of the read-capping nucleotide analog that is incorporated at the terminal 3′ end of individual capped first sequencing read products is not cleaved or removed. In some embodiments, the retained plurality of capped first sequencing read products are not denatured from the template molecules with heat and/or with a chemical denaturation reagent. In some embodiments, the retained plurality of capped first sequencing read products are not degraded with an enzyme having a 5′ to 3′ exonuclease activity (e.g., T7 exonuclease or lambda exonuclease). In some embodiments, the retained plurality of capped first sequencing read products are not degraded with an enzyme having a 3′ to 5′ exonuclease activity (e.g., E. coli exonuclease I or E. coli exonuclease III).

In some embodiments, step (b) comprises: hybridizing individual second sequencing primers to second universal sequencing primer binding sites located on individual nucleic acid template molecules in the plurality of nucleic acid template molecules and conducting one or more second sequencing reactions. In some embodiments, hybridizing individual second sequencing primers to second universal sequencing primer binding sites generates one or more nucleic acid duplexes. In some embodiments, the second forward sequencing primers comprise soluble oligonucleotides that hybridize to at least a portion of a universal binding sequence which is proximal or juxtapositioned to the second region of the nucleic acid template molecule to be sequenced. In some embodiments, the first and second sequencing primers hybridize to different and non-overlapping regions of the same nucleic acid template molecule.

In some embodiments, the sequencing of the second region of step (b) comprises contacting the plurality of nucleic acid template molecules with a plurality of second forward sequencing primers under conditions suitable to hybridize at least one of the second forward sequencing primers to at least one of the second forward sequencing primer binding sites present on each of the nucleic acid template molecules, and conducting one or more second forward sequencing reactions using one or more types of sequencing polymerases, and a plurality of nucleotide reagents (e.g., nucleotides and/or multivalent molecules). In some embodiments, the template molecules are immobilized. In some embodiments, the plurality of second forward sequencing primers are soluble (i.e., in solution). In some embodiments, the second forward sequencing primers comprise soluble oligonucleotides that can hybridize to at least a region of a universal binding sequence which is proximal or juxtapositioned to the second portion of the nucleic acid template molecule to be sequenced. Each of the second forward sequencing reactions can generate a plurality of second forward extension duplexes, each duplex comprising a second forward sequencing read product hybridized to nucleic acid template molecule. In some embodiments, each of the second forward sequence read products comprises a second forward sequencing primer joined to a polynucleotide comprising a sequence that is complementary to a second region of the nucleic acid template molecule to be sequences. A second forward sequencing primer binding site in a nucleic acid template molecule can be hybridized to a second forward sequencing primer and can undergo a sequencing reaction. In some embodiments, the nucleic acid template molecule in the plurality of template molecules is an concatemer template molecule. In some embodiments, the nucleic acid template molecule in the plurality of template molecules is immobilized to a support. In some embodiments, a concatemer template molecule comprises multiple copies of the second forward sequencing primer binding site, wherein each second forward sequencing primer binding site is capable of hybridizing to a second forward sequencing primer. A concatemer template molecule can undergo two or more sequence reactions, where each sequencing reaction is initiated from a different second forward sequencing primer, and each second forward sequencing primer is hybridized to a different second forward sequencing primer binding site (e.g., see FIG. 16B). In some embodiments, the second forward sequencing primers comprise 3′ OH extendible ends. In some embodiments, the second forward sequencing primers comprise a 3′ blocking moiety which can be removed to generate a 3′ OH extendible end. In some embodiments, the second forward sequencing primers lack a nucleotide having a scissile moiety. In some embodiments, the plurality of nucleotide reagents comprises a plurality of nucleotides (or analogs thereof) labeled with a detectable reporter moiety. In some embodiments, the plurality of nucleotide reagents comprises a plurality of multivalent molecules each having a core attached to multiple nucleotide arms, and individual nucleotide arms are attached to a nucleotide unit. In some embodiments the multivalent molecules are labeled with a detectable reporter moiety. In some embodiments, the core is labeled with a detectable reporter moiety. In some embodiments, at least one nucleotide arm and/or at least one nucleotide unit is labeled with a detectable reporter moiety. In some embodiments, the detectable reporter moiety comprises a fluorophore. An illustrative nucleotide arm is shown in FIG. 23, and illustrative multivalent molecules are shown in FIGS. 19-22.

In some embodiments, step (b) comprises: optionally conducting a plurality of second capping reactions, wherein individual capping reactions comprise incorporating a read-capping nucleotide analog into the terminal 3′ end of individual second sequencing read products, thereby generating a plurality of capped second sequencing read products. In some embodiments, the plurality of second capping reactions comprise polymerase-catalyzed nucleotide incorporation reactions. In some embodiments, the plurality of second capping reactions comprises contacting the plurality of second sequencing read products with a plurality of polymerases and a plurality of read-capping nucleotide analogs under a condition suitable for incorporating a read-capping nucleotide analog into the terminal 3′ end of individual second sequencing read products, thereby generating a plurality of capped second sequencing read products. In some embodiments, the plurality of polymerases comprises a sequencing polymerase that can catalyze incorporation of a nucleotide analog comprising a base-linked terminator moiety. In some embodiments, one or more read-capping nucleotide analogs are incorporated into the terminal 3′ end of individual second sequencing read products.

In some embodiments, in step (b), the read-capping nucleotide analog comprises (i) a heterocyclic base, (ii) a sugar, and (iii) a polyphosphate chain, wherein the heterocyclic base is linked to a terminating moiety that blocks binding of a multivalent molecule to the terminal 3′ ends of a capped second sequencing read product. In some embodiments, the read-capping nucleotide analog further comprises a sugar moiety with an extendible 3′OH group. In some embodiments, the read-capping nucleotide analog further comprises a sugar moiety with a non-extendible chain terminating group at the 3′ position. In some embodiments, the sugar comprises 5 carbons, i.e. a pentose sugar. In some embodiments, the sugar comprises a ribose or deoxyribose. Illustrative read-capping nucleotide analogs are shown in FIGS. 8A-8J and 9, and are described in more detail below.

In some embodiments, in step (b), a read-capping nucleotide analog comprises a heterocyclic base which is linked to a terminating moiety that blocks the binding of a multivalent molecule to the terminal 3′ ends of a capped second sequencing read product. In some embodiments, individual multivalent molecules comprises a core attached to multiple nucleotide arms, and individual nucleotide arms are attached to a nucleotide unit. In some embodiments, the base-linked terminating moiety also blocks polymerase-catalyzed incorporation of a subsequent nucleotide (e.g., a canonical nucleotide or a chain terminating nucleotide) into the capped second sequencing read product.

In some embodiments, step (b) comprises: retaining the plurality of capped second sequencing read products which are hybridized to the nucleic acid template molecules. In some embodiments, the terminating moiety of the read-capping nucleotide analog that is incorporated at the terminal 3′ end of individual capped second sequencing read products is not cleaved or removed. In some embodiments, the retained plurality of capped second sequencing read products are not denatured from the template molecules with heat and/or with a chemical denaturation reagent. In some embodiments, the retained plurality of capped second sequencing read products are not degraded with an enzyme having a 5′ to 3′ exonuclease activity (e.g., T7 exonuclease or lambda exonuclease). In some embodiments, the retained plurality of capped second sequencing read products are not degraded with an enzyme having a 3′ to 5′ exonuclease activity (e.g., E. coli exonuclease I or E. coli exonuclease III).

In some embodiments, the pairwise sequencing method comprises step (c): retaining the plurality of nucleic acid template molecules, which are single stranded, and replacing the plurality of first forward sequencing read products and the plurality of second forward sequencing read products with a plurality of forward extension strands that are hybridized to the nucleic acid template molecules. In some embodiments, the pairwise sequencing method comprises step (c): retaining the plurality of nucleic acid template molecules, which are single stranded, and replacing the plurality of capped first sequencing read products and the plurality of capped second sequencing read products with a plurality of forward extension strands that are hybridized to the nucleic acid template molecules. In some embodiments, the nucleic acid template molecules that are retained are immobilized to the support.

In some embodiments, the plurality of first, and optionally second, forward sequencing read products have extendible 3′ terminal ends, which can be removed and replaced with a plurality of forward extension strands by conducting a primer extension reaction (see FIGS. 16C and 16D). The strand replacement step is sometimes referred to as “second strand synthesis” or “pairwise turn.” Described below are different embodiments for replacing the first, and optionally second, forward sequencing read products by conducting primer extension reactions.

In some embodiments, step (c) comprises contacting at least one first forward sequencing read product with a plurality of strand displacing polymerases and a plurality of nucleotides, in the absence of soluble amplification primers and under conditions suitable to conduct a strand displacing primer extension reaction using the at least one first forward sequencing read product to initiate the primer extension reaction from at least one first forward sequencing read product, thereby generating a nascent forward extension strand that is covalently joined to the first forward sequencing read product, wherein the forward extension strand is hybridized to the nucleic acid template molecule. For example, the 3′ end of a first forward sequencing read product can serve as a primer for the strand displacing polymerase. The strand displacing polymerase can extend the first forward sequencing read product, and displace downstream first forward sequencing read products while synthesizing a nascent forward extended strand, which in turn replaces a downstream first forward sequencing read product from the template molecule (see, e.g., FIG. 16C). The newly extended nascent strand may be covalently joined to a first forward sequencing read product. The immobilized template molecules may be retained.

In some embodiments, step (c) can optionally include a plurality of compaction oligonucleotides and/or hexamine (e.g., cobalt hexamine III), which are used to generate the forward extension strands. Individual forward extension strands can collapse into a nanoball having a more compact size and/or shape compared to a nanoball generated from a primer extension reaction conducted without compaction oligonucleotides and/or hexamine (e.g., cobalt hexamine III). Inclusion of compaction oligonucleotides and/or hexamine (e.g., cobalt hexamine III) in the primer extension reaction can improve FWHM (full width half maximum) of a spot image of the nanoball. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 um or smaller.

Examples of strand displacing polymerases include phi29 DNA polymerase, large fragment of Bst DNA polymerase, large fragment of Bsu DNA polymerase (exo-), Bca DNA polymerase (exo-), Klenow fragment of E. coli DNA polymerase, T5 polymerase, M-MuLV reverse transcriptase, HIV viral reverse transcriptase, Deep Vent® DNA polymerase and KOD DNA polymerase. The phi29 DNA polymerase can be wild type phi29 DNA polymerase (e.g., MagniPhi™ from Expedeon), or variant EquiPhi29™ DNA polymerase (e.g., from ThermoFisher® Scientific), or chimeric QualiPhi™ DNA polymerase (e.g., from 4basebio).

In some embodiments, step (c) comprises: (i) removing the plurality of first sequencing read products and/or the plurality of second sequencing read products, and/or removing the plurality of first capped sequencing read products and the plurality of second capped sequencing read products, while retaining the template molecules; and (ii) contacting the plurality of retained template molecules with an additional plurality of forward sequencing primers (e.g., a third plurality of forward sequencing primers), a plurality of nucleotides and a plurality of primer extension polymerases, under conditions suitable to hybridize the plurality of forward sequencing primer to the plurality of retained template molecules and suitable for conducting polymerase-catalyzed primer extension reactions, thereby generating a plurality of forward extension strands, wherein the sequencing primers hybridize with the forward sequencing primer binding site present in the retained template molecules (FIG. 16D). The primer extension reaction can optionally comprise a plurality of compaction oligonucleotides and/or hexamine (e.g., cobalt hexamine III) to generate forward extension strands. A forward extension strand can collapse into a nanoball having a more compact size and/or shape compared to a nanoball generated from a primer extension reaction conducted without compaction oligonucleotides and/or hexamine (e.g., cobalt hexamine III). Inclusion of compaction oligonucleotides and/or hexamine (e.g., cobalt hexamine III) in the primer extension reaction can improve FWHM (full width half maximum) of a spot image of the nanoball. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 um or smaller.

In some embodiments, the nucleic acid template molecules are immobilized. In some embodiments, the sequencing primers, i.e. any of the first, second and/or third pluralities of forward sequencing primers, are soluble (i.e., in solution).

In some embodiments, step (c) comprises hybridizing the plurality of nucleic acid template molecules which have been retained with a plurality of sequencing primers in the presence of a primer extension polymerase, a plurality of nucleotides, and a high efficiency hybridization buffer. In some embodiment, the high efficiency hybridization buffer comprises: (i) a first polar aprotic solvent having a dielectric constant that is no greater than 40 and having a polarity index of 4-9; (ii) a second polar aprotic solvent having a dielectric constant that is no greater than 115 and is present in the hybridization buffer formulation in an amount effective to denature double-stranded nucleic acids; (iii) a pH buffer system that maintains the pH of the hybridization buffer formulation in a range of about 4-8; and (iv) a crowding agent in an amount sufficient to enhance or facilitate molecular crowding. In some embodiments, the high efficiency hybridization buffer comprises: (i) the first polar aprotic solvent comprises acetonitrile at 25-50% by volume of the hybridization buffer; (ii) the second polar aprotic solvent comprises formamide at 5-10% by volume of the hybridization buffer; (iii) the pH buffer system comprises 2-(N-morpholino)ethanesulfonic acid (MES) at a pH of 5-6.5; and (iv) the crowding agent comprises polyethylene glycol (PEG) at 5-35% by volume of the hybridization buffer. In some embodiments, the high efficiency hybridization buffer further comprises betaine.

In some embodiments, step (c) comprises: (i) removing the plurality of first forward sequencing read products and/or the plurality of second forward sequencing read products while retaining the nucleic acid template molecules and/or removing the plurality of capped first sequencing read products and the plurality of capped second sequencing read products while retaining the nucleic acid template molecules; and (ii) contacting the plurality of nucleic acid template molecules with a plurality of amplification primers, a plurality of nucleotides and a plurality of primer extension polymerases, under conditions suitable to hybridize the plurality of amplification primers to the plurality of nucleic acid template molecules and suitable for conducting polymerase-catalyzed primer extension reactions, thereby generating a plurality of forward extension strands, wherein the amplification primers hybridize with the amplification primer binding sequence located in the nucleic acid template molecules. In some embodiments, the nucleic acid template molecules that are retained are immobilized to a support. In some embodiments, the amplification primers are soluble (i.e., in solution). The primer extension reaction can optionally comprise a plurality of compaction oligonucleotides and/or hexamine (e.g., cobalt hexamine III) to generate forward extension strands. Individual forward extension strands can collapse into a nanoball having a more compact size and/or shape compared to a nanoball generated from a primer extension reaction conducted without compaction oligonucleotides and/or hexamine (e.g., cobalt hexamine III). Inclusion of compaction oligonucleotides and/or hexamine (e.g., cobalt hexamine III) in the primer extension reaction can improve FWHM (full width half maximum) of a spot image of the nanoball. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 um or smaller.

In some embodiments, step (c) comprises hybridizing the plurality of nucleic acid template molecules that have been retained with the plurality of amplification primers in the presence of a primer extension polymerase, a plurality of nucleotides, and a high efficiency hybridization buffer. In some embodiment, the high efficiency hybridization buffer comprises: (i) a first polar aprotic solvent having a dielectric constant that is no greater than 40 and having a polarity index of 4-9; (ii) a second polar aprotic solvent having a dielectric constant that is no greater than 115 and is present in the hybridization buffer formulation in an amount effective to denature double-stranded nucleic acids; (iii) a pH buffer system that maintains the pH of the hybridization buffer formulation in a range of about 4-8; and (iv) a crowding agent in an amount sufficient to enhance or facilitate molecular crowding. In some embodiments, the high efficiency hybridization buffer comprises: (i) the first polar aprotic solvent comprises acetonitrile at 25-50% by volume of the hybridization buffer; (ii) the second polar aprotic solvent comprises formamide at 5-10% by volume of the hybridization buffer; (iii) the pH buffer system comprises 2-(N-morpholino)ethanesulfonic acid (MES) at a pH of 5-6.5; and (iv) the crowding agent comprises polyethylene glycol (PEG) at 5-35% by volume of the hybridization buffer. In some embodiments, the high efficiency hybridization buffer further comprises betaine.

In some embodiments, step (c) comprises: (i) contacting the plurality of nucleic acid template molecules (e.g., that have been retained by immobilization as described above) with a plurality of soluble primers that hybridize to a region that is upstream of at least one of the capped sequencing read products hybridized to a nucleic acid template molecule, a plurality of nucleotides and a plurality of strand displacing polymerases, under conditions suitable to hybridize the plurality of soluble upstream primers to the plurality of nucleic acid template molecules, and (ii) conducting one or more polymerase-catalyzed stand displacing reactions, thereby generating a plurality of forward extension strands. The strand displacing polymerase can displace the downstream capped sequencing read product. The nucleic template molecules may be retained. The nucleic template molecules may be immobilized. The strand-displacing reaction can optionally comprise a plurality of compaction oligonucleotides and/or hexamine (e.g., cobalt hexamine III) to generate forward extension strands. A forward extension strand can collapse into a nanoball having a more compact size and/or shape compared to a nanoball generated from a strand-displacing reaction conducted without compaction oligonucleotides and/or hexamine (e.g., cobalt hexamine III). Inclusion of compaction oligonucleotides and/or hexamine (e.g., cobalt hexamine III) in the strand-displacing reaction can improve FWHM (full width half maximum) of a spot image of the nanoball. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 um or smaller.

In some embodiments, step (c) comprises contacting a plurality of nucleic acid template molecules that have been retained with a plurality of soluble upstream primers in the presence of a strand-displacing polymerase, a plurality of nucleotides, and a high efficiency hybridization buffer. The nucleic template molecules may be retained. In some embodiments, the nucleic acid template molecules are immobilized. In some embodiment, the high efficiency hybridization buffer comprises: (i) a first polar aprotic solvent having a dielectric constant that is no greater than 40 and having a polarity index of 4-9; (ii) a second polar aprotic solvent having a dielectric constant that is no greater than 115 and is present in the hybridization buffer formulation in an amount effective to denature double-stranded nucleic acids; (iii) a pH buffer system that maintains the pH of the hybridization buffer formulation in a range of about 4-8; and (iv) a crowding agent in an amount sufficient to enhance or facilitate molecular crowding. In some embodiments, the high efficiency hybridization buffer comprises: (i) the first polar aprotic solvent comprises acetonitrile at 25-50% by volume of the hybridization buffer; (ii) the second polar aprotic solvent comprises formamide at 5-10% by volume of the hybridization buffer; (iii) the pH buffer system comprises 2-(N-morpholino)ethanesulfonic acid (MES) at a pH of 5-6.5; and (iv) the crowding agent comprises polyethylene glycol (PEG) at 5-35% by volume of the hybridization buffer. In some embodiments, the high efficiency hybridization buffer further comprises betaine.

In some embodiments, the primer extension polymerase of step (c) comprises a high fidelity polymerase. In some embodiments, the primer extension polymerase of step (c) comprises a DNA polymerase capable of catalyzing a primer extension reaction using a uracil-containing template molecule (e.g., a uracil-tolerant polymerase). Illustrative polymerases include, but are not limited to, Q5U® Hot Start high-fidelity DNA polymerase (e.g., catalog #M0515S from New England Biolabs®), Taq DNA polymerase, One Taq DNA polymerase (e.g., mixture of Taq and Deep Vent® DNA polymerases, catalog #M0480S from New England Biolabs), LongAmp® Taq DNA polymerase (e.g., catalog #M0323S from New England Biolabs), Epimark® Hot Start Taq DNA polymerase (e.g., catalog #M0490S from New England Biolabs), Bst DNA polymerase (e.g., large fragment, catalog #M0275S from New England Biolabs), Bsu DNA polymerase (e.g., large fragment, catalog #M0330S from New England Biolabs), Phi29 DNA polymerase (e.g., catalog #M 0269S from New England Biolabs), E. coli DNA polymerase (e.g., catalog #M0209S from New England Biolabs), Therminator™ DNA polymerase (e.g., catalog #M0261S from New England Biolabs), Vent® DNA polymerase and Deep Vent® DNA polymerase.

In some embodiments, the pairwise sequencing method comprises step (d): removing the plurality of nucleic acid template molecules retained at step (c) by generating abasic sites in the nucleic acid template molecules at the nucleotide(s) having the scissile moiety and generating gaps at the abasic sites to generate a plurality of gap-containing nucleic acid template molecules, while retaining the plurality of forward extension strands and retaining the plurality of immobilized surface primers (see e.g., FIG. 16E). In some embodiments, the nucleic acid template molecules are immobilized. In some embodiments, the template molecules are single stranded.

The abasic sites are generated on nucleic acid template molecules that contain nucleotides having scissile moieties, which can be single stranded. In some embodiments, a scissile moiety present in the nucleic acid template molecules comprises uridine, 8-oxo-7,8-dihydroguanine (e.g., 8oxoG) or deoxyinosine. The abasic sites can then be removed to generate a plurality of gap-containing single stranded nucleic acid template molecules while retaining the plurality of forward extension strands. The abasic sites can be generated by contacting the plurality of nucleic acid template molecules with an enzyme that removes the nucleobase from the nucleotide having a scissile moiety. A uracil in a nucleic acid template strand can be converted to an abasic site using uracil DNA glycosylase (UDG). An 8oxOG in a nucleic acid template strand can be converted to an abasic site using FPG glycosylase. A deoxyinosine in a nucleic acid template strand can be converted to an abasic site using AlkA glycosylase.

In some embodiments of step (d), the gaps can be generated by contacting the abasic sites in the nucleic acid template molecules with an enzyme or a mixture of enzymes having lyase activity that breaks the phosphodiester backbone at the 5′ and 3′ sides of the abasic site to release the base-free deoxyribose and generate a gap (see e.g., FIG. 16E). The abasic sites can be removed, for example, using AP lyase, Endo IV endonuclease, FPG glycosylase/AP lyase, or Endo VIII glycosylase/AP lyase. In some embodiments, generating the abasic sites and removal of the abasic sites to generate gaps can be achieved using a mixture of uracil DNA glycosylase and DNA glycosylase-lyase endonuclease VIII, for example USER (Uracil-Specific Excision Reagent Enzyme from New England Biolabs) or thermolabile USER (also from New England Biolabs).

In some embodiments of step (d), the plurality of gap-containing nucleic acid template molecules can be removed from the immobilized surface primers using an enzyme, chemical and/or heat. After the gap-removal procedure, the forward extension strands are retained, thereby generating a plurality of retained forward extension strands (e.g., see FIG. 16F) and are hybridized to the immobilized surface primers.

For example, the plurality of gap-containing nucleic acid template molecules can be enzymatically degraded using a 5′ to 3′ double-stranded DNA exonuclease, such as, for example, T7 exonuclease (e.g., from New England Biolabs, catalog #M0263S) or lambda exonuclease (e.g., from New England Biolabs, catalog No. M0262S). When a 5′ to 3′ double-stranded DNA exonuclease is used for removing gap-containing nucleic template molecules from the immobilized surface primers, then each of the plurality of soluble amplification primers in step (c) can comprise at least one phosphorothioate diester bond at the 5′ end, which can render the soluble amplification primer resistant to exonuclease degradation. In some embodiments, a soluble amplification primer in step (c) comprises 2-5 or more consecutive phosphorothioate diester bonds at the 5′ ends. In some embodiments, a soluble amplification primer in step (c) comprises at least one ribonucleotide and/or at least one 2′-O-methyl or 2′-O-methoxyethyl (MOE) nucleotide which can render the forward sequencing primer resistant to exonuclease degradation.

In some embodiments, the plurality of gap-containing template molecules can be removed from the surface primers immobilized to the support using a chemical reagent that favors nucleic acid denaturation. The denaturation reagent can comprise formamide, acetonitrile, guanidinium chloride and/or a buffering agent (e.g., Tris-HCl, MES, HEPES, or the like) or a combination thereof.

In some embodiments, the plurality of gap-containing template molecules can be removed using an elevated temperature (e.g., heat) with or without a nucleic acid denaturation reagent. The gap-containing template molecules can be subjected to a temperature of about 45-50 °C, or about 50-60 °C, or about 60-70 °C, or about 70-80 °C, or about 80-90 °C, or about 90-95 °C, or higher.

In some embodiments, the plurality of gap-containing template molecules can be removed using formamide (e.g., 40-100% formamide) at a temperature of about 65 °C for about 3 minutes, and washing with a reagent comprising about 50 mM NaCl or equivalent ionic strength and having a pH of about 6.5-8.5.

In some embodiments, the pairwise sequencing method comprises step (e): sequencing the plurality of retained forward extension strands, thereby generating a plurality of first reverse sequencing read products. In some embodiments, the sequencing of step (e) comprises contacting the plurality of retained forward extension strands with a plurality of soluble reverse sequencing primers under a condition suitable to hybridize a reverse sequencing primer to a reverse sequencing primer binding site located on a retained forward extension strand, and subsequently conducting sequencing reactions, wherein the reverse sequencing reactions generate a plurality of first reverse sequencing read products (see e.g., FIG. 16G). The first reverse sequencing read products may be hybridized to the retained forward extension strand. The retained forward extension strand may be hybridized to a first surface primer. In some embodiments, the first reverse sequencing read product is not hybridized to the first surface primer, nor covalently joined to the first surface primer. Therefore, in such embodiments, the first reverse sequencing read products are not immobilized to the support.

For the sake of simplicity, FIGS. 16F-16G show illustrative retained forward extension strands, each having two copies of the sequence of interest and various universal primer binding sites. The skilled artisan will appreciate that the retained forward extension strand can comprise two or more tandem copies containing the sequence of interest and various universal primer binding sites. Therefore, the reverse sequencing reaction can generate a plurality of first reverse sequencing read products that are hybridized to the same retained forward extension strand.

In some embodiments, step (e) comprises contacting the plurality of soluble reverse sequencing primers and the plurality of retained forward extension strands with a high efficiency hybridization buffer. In some embodiments, the high efficiency hybridization buffer comprises: (i) a first polar aprotic solvent having a dielectric constant that is no greater than 40 and having a polarity index of 4-9; (ii) a second polar aprotic solvent having a dielectric constant that is no greater than 115 and is present in the hybridization buffer formulation in an amount effective to denature double-stranded nucleic acids; (iii) a pH buffer system that maintains the pH of the hybridization buffer formulation in a range of about 4-8; and (iv) a crowding agent in an amount sufficient to enhance or facilitate molecular crowding. In some embodiments, the high efficiency hybridization buffer comprises: (i) the first polar aprotic solvent comprises acetonitrile at 25-50% by volume of the hybridization buffer; (ii) the second polar aprotic solvent comprises formamide at 5-10% by volume of the hybridization buffer; (iii) the pH buffer system comprises 2-(N-morpholino)ethanesulfonic acid (MES) at a pH of 5-6.5; and (iv) the crowding agent comprises polyethylene glycol (PEG) at 5-35% by volume of the hybridization buffer. In some embodiments, the high efficiency hybridization buffer further comprises betaine.

In alternative embodiments of step (e) the surface primer that is immobilized to the support serves as a sequencing primer, and step (e) comprises conducting sequencing reactions to generate a plurality of first reverse sequencing reads products.

In some embodiments, the reverse sequencing reaction of step (e) comprises contacting the plurality of soluble reverse sequencing primers with the reverse sequencing primer binding sites present on each of the retained forward extension strands, one or more types of sequencing polymerases, and a plurality of nucleotides or a plurality of multivalent molecules. In some embodiments, a soluble reverse sequencing primer comprises 3′ OH extendible ends. In some embodiments, a soluble reverse sequencing primer comprises a 3′ blocking moiety which can be removed to generate a 3′ OH extendible end. In some embodiments, a soluble reverse sequencing primer lacks a nucleotide having a scissile moiety. The sequencing reactions that employ nucleotides and/or multivalent molecules are described in more detail below. The reverse sequencing reactions can generate a plurality of first reverse sequencing read products. In some embodiments, a retained forward extension strand comprises multiple copies of the reverse sequencing primer binding sites, wherein each reverse sequencing primer binding site is capable of hybridizing to a reverse sequencing primer. A reverse sequencing primer binding site can be hybridized to a reverse sequencing primer and can undergo a sequencing reaction. Thus, an individual retained forward extension strand can undergo two or more sequence reactions, where each sequencing reaction is initiated from a reverse sequencing primer that is hybridized to a reverse sequencing primer binding site (e.g., see FIG. 16G). In some embodiments, the sequencing reactions comprise a plurality of nucleotides (or analogs thereof) labeled with a detectable reporter moiety. In some embodiments, the sequencing reaction comprise a plurality of multivalent molecules having nucleotide units, where the multivalent molecules are labeled with a detectable reporter moiety. In some embodiments, the detectable reporter moiety comprises a fluorophore.

In some embodiments, at least one washing step can be conducted after any of steps (a)-(e). The washing step can be conducted with a wash buffer comprising a pH buffering agent, a metal chelating agent, a salt, and/or a detergent.

In some embodiments, the pH buffering compound in the wash buffer is Tris, Tris-HCl, Tricine, Bicine, Bis-Tris propane, HEPES, MES, MOPS, MOPSO, BES, TES, CAPS, TAPS, TAPSO, ACES, PIPES, ethanolamine (a.k.a 2-amino methanol; MEA), a citrate compound, a citrate mixture, NaOH, KOH, or any combination thereof. In some embodiments, the pH buffering agent can be present in the wash buffer at a concentration of about 1-100 mM. or about 10-50 mM, or about 10-25 mM. In some embodiments, the pH of the pH buffering agent which is present in any of the reagents described here in can be adjusted to a pH of about 4-9, or a pH of about 5-9, or a pH of about 5-8.

In some embodiments, the metal chelating agent in the wash buffer comprises EDTA (ethylenediaminetetraacetic acid), EGTA (ethylene glycol tetraacetic acid), HEDTA (hydroxyethylethylenediaminetriacetic acid), DPTA (diethylene triamine pentaacetic acid), NTA (N,N-bis(carboxymethyl)glycine), citrate anhydrous, sodium citrate, calcium citrate, ammonium citrate, ammonium bicitrate, citric acid, potassium citrate, magnesium citrate, or any combination thereof. In some embodiments, the wash buffer comprises a chelating agent at a concentration of about 0.01-50 mM, or about 0.1-20 mM, or about 0.2-10 mM.

In some embodiments, the salt in the wash buffer comprises NaCl, KCl, NH₂SO₄, potassium glutamate or any combination thereof. In some embodiments, the detergent comprises an ionic detergent such as SDS (sodium dodecyl sulfate). The wash buffer can comprise a monovalent salt at a concentration of about 25-500 mM, or about 50 -250 mM, or about 100-200 mM.

In some embodiments, the detergent in the wash buffer comprises a non-ionic detergent such as Triton X-100, Tween 20, Tween 80 or Nonidet P-40. In some embodiments, the detergent comprises a zwitterionic detergent such as CHAPS (3-[(3-cholamidopropyl)dimethylammonio]-1-propanesulfonate) or N-Dodecyl-N, N-dimethyl-3-amonio-1-propanesulfate (DetX). In some embodiments, the detergent comprises LDS (lithium dodecyl sulfate), sodium taurodeoxycholate, sodium taurocholate, sodium glycocholate, sodium deoxycholate or sodium cholate. In some embodiments, the detergent is present in the wash buffer at a concentration of about 0.01-0.05%, or about 0.05-0.1%, or about 0.1-0.15%, or about 0.15-0.2%, or about 0.2-0.25%.

The present disclosure provides read-capping nucleotide analogs which can be used in any of the methods described herein, including any of the methods for sequencing two or more regions of a nucleic acid template molecule and any of the pairwise sequencing methods.

In some embodiments, the read-capping nucleotide analogs comprise: (i) a heterocyclic base, (ii) a ribose sugar, and (iii) a polyphosphate chain, wherein the heterocyclic base is linked to a terminating moiety (e.g., base-linked chain terminating moiety). In some embodiments, read-capping nucleotide analog can be incorporated into a terminal 3′ end of a primer, or incorporated into the terminal 3′ end of a nascent primer, in a polymerase-catalyzed primer extension reaction to generate a capped primer product.

In some embodiments of the methods described herein, the read-capping nucleotide analog, once incorporated, can block polymerase-mediated binding of a nucleotide reagent to the terminal 3′ ends of the read capped primer product, where the nucleotide reagent comprises a multivalent molecule. In some embodiments, a multivalent molecule comprises a core attached to multiple nucleotide arms and individual nucleotide arms are attached to nucleotide units. In some embodiments, a multivalent molecule comprises (1) a core; and (2) a plurality of nucleotide arms each comprising (i) a core attachment moiety and (ii) a spacer comprising, (a) a linker, and (b) a nucleotide unit, wherein the core is attached to the plurality of nucleotide arms, wherein the spacer is attached to the linker, wherein the linker is attached to the nucleotide unit (e.g., see FIGS. 19-23).

In some embodiments, the incorporated read-capping nucleotide analog can block polymerase-catalyzed incorporation of a nucleotide reagent to the terminal 3′ ends of the read capped primer product, wherein the nucleotide reagent is a canonical nucleotide or a chain terminating nucleotide.

In some embodiments of the methods described herein, the read-capping nucleotide analog comprises: (i) a heterocyclic base, (ii) a ribose sugar, and (iii) at least one phosphate group, wherein the heterocyclic base is linked to a terminating moiety. In some embodiments, the terminating moiety is linked to the heterocyclic base by a linker that is non-cleavable. In some embodiments, the terminating moiety is linked to the heterocyclic base by a linker that is cleavable In some embodiments, the read-capping nucleotide analogs can be non-labeled. In some embodiments, the read-capping nucleotide analogs can be labeled with a detectable reporter moiety. FIGS. 8A-8J and 9 show illustrative read-capping nucleotide analogs.

In some embodiments of the methods described herein, the read-capping nucleotide analogs comprise a heterocyclic base comprising a substituted nitrogen-containing parent heteroaromatic ring. In some embodiments, the substituted nitrogen-containing parent heteroaromatic ring, is naturally-occurring, substituted, modified, or engineered variants. In some embodiments, the base of a read-capping nucleotide analog is capable of forming Watson-Crick and/or Hoogstein hydrogen bonds with an appropriate complementary base. Illustrative bases that may be used in the read-capping nucleotide analogs include, but are not limited to, purines and pyrimidines such as: 2-aminopurine; 2,6-diaminopurine; adenine (A); ethenoadenine; N⁶-Δ²-isopentenyladenine (6iA); N⁶-Δ²-isopentenyl-2-methylthioadenine (2ms6iA); N⁶-methyladenine; guanine (G); isoguanine; N²-dimethylguanine (dmG); 7-methylguanine (7mG); 2-thiopyrimidine; 6-thioguanine (6sG); hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O4-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional illustrative bases can be found in Fasman, 1989, in “Practical Handbook of Biochemistry and Molecular Biology”, pp. 385-394, CRC Press, Boca Raton, Fla.

In some embodiments of the methods described herein, the read-capping nucleotide analogs comprise a base-linked terminating moiety which be linked to the nucleobase by an alkyl linkage. In some embodiments, the linkage comprises an alkyl group. In some embodiments, the linkage comprises an alkenyl group. In some embodiments, the linkage comprises an alkynyl group.

In some embodiments, the base-linked terminating moiety comprises a polymer moiety. In some embodiments, the base-linked terminating moiety comprises a polyether moiety. In some embodiments, the base-linked terminating moiety comprises polyethylene glycol. In some embodiments, the base-linked terminating moiety comprises polypropylene glycol. In some embodiments, the base-linked terminating moiety comprises polyvinyl acetate. In some embodiments, the base-linked terminating moiety comprises apolylactic acid. In some embodiments, the base-linked terminating moiety comprises polyglycolic acid. In some embodiments, the base-linked terminating moiety comprises a polyamide moiety. In some embodiments, the base-linked terminating moiety comprises a polyester moiety.

In some embodiments, the base-linked terminating moiety comprises a poly(glycerol) moiety. In some embodiments, the base-linked terminating moiety comprises a poly(oxazoline) moiety. In some embodiments, the base-linked terminating moiety comprises a poly(hydroxypropyl methacrylate) moiety (PHPMA). In some embodiments, the base-linked terminating moiety comprises a poly(2-hydroxyethyl methacrylate) moiety (PHEMA). In some embodiments, the base-linked terminating moiety comprises a poly(N-(2-hydroxypropyl) methacrylamide) moiety (HPMA). In some embodiments, the base-linked terminating moiety comprises a poly(vinylpyrrolidone) moiety (PVP). In some embodiments, the base-linked terminating moiety comprises a poly(N,N-dimethyl acrylamide) moiety (PDMA). In some embodiments, the base-linked terminating moiety comprises a poly(N-acryloylmorpholine) moiety (PACM).

In some embodiments, the base-linked terminating moiety comprises a synthetic zwitterionic moiety such as a poly(carboxybetaine acrylamide) moiety, a poly(carboxybetaine methacrylate) moiety, a poly(sulfobetaine methacrylate) moiety or a poly(methacryloyloxyethyl phosphorylcholine) moiety.

In some embodiments, the base-linked terminating moiety comprises a polyglutamate moiety. In some embodiments, the base-linked terminating moiety comprises a polyaspartate moiety. In some embodiments, the base-linked terminating moiety comprises a polylysine moiety. In some embodiments, the base-linked terminating moiety comprises a polyethyeleneimine moiety. In some embodiments, the base-linked terminating moiety comprises a polysialic acid moiety.

In some embodiments, the base-linked terminating moiety comprises a linker, for example an 11 atom linker, a 16 atom linker, a 23 atom linker or an N3 linker, examples of which are shown in FIG. 24. In some embodiments, the base-linked terminating moiety comprises a polymer linker, for example any one of Linkers 1-9 shown in FIG. 25.

In some embodiments, the base-linked terminating moiety comprises a propargylamino moiety, an allylamino moiety, a propylamino moiety, an ethylmercapto moiety, a hydroxymethyl moiety, or an arylmercapto moiety (e.g., see FIGS. 8A-8J). In some embodiments, the base-linked terminating moiety comprises a 1-X-1H-1,2,3-triazol-4-yl moiety, a 5-X-1H-1,2,3-triazol-1-methyl moiety, a hydrazone moiety or an O-alkyl oxime moiety (e.g., see FIGS. 8A-8J).

In some embodiments, the read-capping nucleotide analog comprises any of the nucleotide analogs shown in FIGS. 8A-8J, wherein “X” comprises a polymer, such as, for example, any polymer described above.

In some embodiments of the methods described herein, the base-linked terminating moiety comprises a hydrophilic polymer of ethylene oxide. In some embodiments, the base-linked terminating moiety comprises a polyethylene glycol (PEG) moiety (e.g., H—(OCH₂CH₂)ₙ—OH, where n can be 1, or at least 2, or at least 5, or at least 10, at least 20, at least 25, at least 50, at least 75, at least 100, at least 200, at least 500, at least 750, at least 1000, or 2000 or more). In some embodiments, the PEG moiety has a molecular weight of about 100-200 Da, 200-300 Da, 300-400 Da, 400-500 Da, 1K Da, 2K Da, 3K Da, 4K Da, 5K Da, 10K Da, 15 K Da, 20K Da, 30K Da, 40K Da, 50K Da, or larger molecular weight. In some embodiments, the value of. In some embodiments, the PEG moiety comprises a linear PEG molecule. In some embodiments, the PEG moiety comprises a branched PEG molecule. In some embodiments, the branched PEG moiety molecule comprise 4-8 branches. In some embodiments, the terminating moiety comprises an aromatic group having a six-carbon ring structure. In some embodiments, the PEG moiety can be functionalized with a methoxy group (OCH₃), amino group (NH₂), carboxyl group (COOH) and/or hydroxyl group (OH). For example, the base-linked terminating moiety comprises polyethylene glycol methyl ether (mPEG) (e.g., CH₃(OCH₂CH₂)ₙ—OH).

In some embodiments, the read-capping nucleotide analog comprises any of the nucleotide analogs shown in FIGS. 8A-8J, wherein “X” comprises a PEG moiety or derivative thereof, such as, for example any PEG moiety described above.

Also provided herein are sets of read-capping nucleotides that may be used in the methods described herein. In some embodiments, a set of read-capping nucleotide analogs comprises four types of read-capping nucleotide analogs, including dATP, dGTP, dCTP and dTTP/dUTP, wherein the nucleobase of each type of read-capping nucleotide analog is linked to the same type of terminating moiety. For example, the same type of PEG moiety is linked to the nucleobase of the four types of read-capping nucleotide analogs dATP, dGTP, dCTP and dTTP/dUTP in a set of read-caping nucleotide analogs.

In some embodiments, a set of read-capping nucleotide analogs comprises four types of read-capping nucleotide analogs, including dATP, dGTP, dCTP and dTTP/dUTP, wherein the nucleobase of at least one type of read-capping nucleotide analog is linked to a different terminating moiety than at least one of the remaining three read-capping nucleotide analogs. For example, in such embodiments, at least one type of read-capping nucleotide analog (e.g., dATP) comprises a nucleobase that is linked to a PEG moiety that differs from the PEG moiety of other read-capping nucleotide analogs (e.g., dGTP, dCTP and cTTP/dUTP). In some embodiments, the PEG moiety can differ in length. In some embodiments, the PEG moiety can differ in molecular weight. In some embodiments, the nucleobase can be linked to a PEG moiety by a different linkage (e.g., a different alkyl, alkenyl, or alkynyl linkage). In some embodiments, the nucleobase in each of the four types of read-capping nucleotide analogs can be linked to a PEG moiety having a different functional group, including a methoxy group (OCH₃), amino group (NH₂), carboxyl group (COOH) and/or hydroxyl group (OH).

In some embodiments of the methods described herein, the read-capping nucleotide analog comprises a sugar moiety, such as cyclic moiety (see Ferraro and Gotor 2000 Chem. Rev. 100: 4319-48, which is incorporated herein by reference for examples of cyclic moieties that may be present in the read-capping nucleotide analogs described herein), an acyclic moiety (see Martinez, et al., 1999 Nucleic Acids Research 27: 1271-1274; Martinez, et al., 1997 Bioorganic & Medicinal Chemistry Letters vol. 7: 3013-3016, each of which is incorporated herein by reference for examples of acyclic moieties that may be present in the read-capping nucleotide analogs described herein), or another sugar moiety (see Joeng, et al., 1993 J. Med. Chem. 36: 2627-2638; Kim, et al., 1993 J. Med. Chem. 36: 30-7; Eschenmosser 1999 Science 284:2118-2124; and U.S. Pat. No. 5,558,991, each of which is incorporated herein by reference for examples of sugar moieties that may be present in the read-capping nucleotide analogs described herein). The sugar moiety may be: ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments of the methods described herein, the read-capping nucleotide analog comprises a hydroxyl group at the 3′ sugar position. In some embodiments, the read-capping nucleotide analog comprises a chain terminating moiety at the 3′ sugar position, where the chain terminating moiety comprises H, F, NH₂, or an amino group.

In some embodiments, the read-capping nucleotide analog comprises a sugar moiety comprising a 3′OH group. In some embodiments, the read-capping nucleotide analog comprises a chain terminating moiety at the 2′ or 3′ sugar position. In some embodiments, the chain terminating moiety is removable or cleavable from the 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable or removable from the nucleotide, for example by reacting the chain terminating moiety with a chemical agent, by a pH change, by light or by heat. In some embodiments, chain terminating moieties comprising alkyl, alkenyl, alkynyl or allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, chain terminating moieties comprising aryl or benzyl are cleavable with H₂ Pd/C. In some embodiments, chain terminating moieties comprising amine, amide, keto, isocyanate, phosphate, thio, or disulfide are cleavable with phosphine or with a thiol group including beta-mercaptoethanol or dithiothritol (DTT). In some embodiments, chain terminating moieties comprising carbonate are cleavable with potassium carbonate (K₂CO₃) in MeOH, with triethylamine in pyridine, or with Zn in acetic acid (AcOH). In some embodiments, chain terminating moieties comprising urea or silyl are cleavable with tetrabutylammonium fluoride, pyridine-HF, with ammonium fluoride, or with triethylamine trihydrofluoride. In some embodiments, a chain terminating moiety may be cleavable or removable with nitrous acid. In some embodiments, a chain terminating moiety may be cleavable or removable using a solution comprising nitrite, such as, for example, a combination of nitrite with an acid such as acetic acid, sulfuric acid, or nitric acid. In some further embodiments, said solution may comprise an organic acid.

In some embodiments, the 2′ or 3′ chain terminating moiety of a read-capping nucleotide analog comprises an azide, azido or azidomethyl group. In some embodiments, the chain terminating moiety comprises a 3′-O-azido or 3′-O-azidomethyl group. In some embodiments, the chain terminating moieties comprising azide, azido and azidomethyl group are cleavable or removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the 2′ or 3′ chain terminating moiety of the read-capping nucleotide analogs comprise a 3′-O-amino group or a derivative thereof, which can be cleaved with nitrous acid, through a mechanism utilizing nitrous acid, or using a solution comprising nitrous acid. In some embodiments, the 2′ or 3′ chain terminating moiety of the read-capping nucleotide analogs comprise a 3′-O-aminomethyl group or a derivative thereof, which can be cleaved with nitrous acid, through a mechanism utilizing nitrous acid, or using a solution comprising nitrous acid. In some embodiments, the 2′ or 3′ chain terminating moiety of the read-capping nucleotide analogs comprise a 3′-O-methylamino group, or a derivative thereof, which can be cleaved with nitrous acid, through a mechanism utilizing nitrous acid, or using a solution comprising nitrous acid.

In some embodiments, the chain terminating moiety comprises a 3′-O-amino group or a derivative thereof, which can be cleaved using a solution comprising nitrite. In some embodiments, the chain terminating moiety comprises a 3′-O-aminomethyl group or a derivative thereof, which can be cleaved using a solution comprising nitrite. In some embodiments, the chain terminating moiety comprises a 3′-O-methylamino group, or a derivative thereof, which can be cleaved using a solution comprising nitrite. In some embodiments, for example, nitrite may be combined with an acid such as acetic acid, sulfuric acid, or nitric acid. In some further embodiments, for example, nitrite may be combined with an organic acid such as, for example, formic acid, acetic acid, propionic acid, butyric acid, isobutyric acid, or the like.

In some embodiments, in any of the methods described herein, the read-capping nucleotide analogs comprise a 3′-deoxy nucleotide, 2′,3′-dideoxynucleotide, or a nucleotide that comprises one of the following 3′ modifications: 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments of the methods described herein, the read-capping nucleotide analog comprises a chain of 1, 2, 3, 4, 5, 6, 7, 8, 9, 10 or more phosphorus atoms, wherein the chain is typically attached to the 5′ carbon of the sugar moiety via an ester or phosphoramide linkage. In some embodiments, the nucleotide analog comprises a phosphorus chain in which the phosphorus atoms are linked together with intervening O, S, NH, methylene or ethylene. In some embodiments, the phosphorus atoms in the phosphorus chain comprise substituted side groups such as O, S or BH₃. In some embodiments, the phosphorus chain comprises phosphate groups substituted with analogs, such as phosphoramidate, phosphorothioate, phosphordithioate, and O-methylphosphoroamidite groups.

In some embodiments of the methods described herein, including any of the methods for sequencing two or more regions of a nucleic acid template molecule and any of the pairwise sequencing methods, the sequencing primer comprises an oligonucleotide that is capable of hybridizing with a DNA and/or RNA template molecule to form a duplex molecule. The sequencing primers may comprise natural nucleotides and/or nucleotide analogs. The sequencing primers may comprise recombinant nucleic acid molecules or synthetic nucleic acid molecules. The sequencing primers may have any length, but typically range from 4-50 nucleotides. In some embodiments, the 3′ end of the sequencing primer comprises an extendible 3′ OH moiety, which serves as a nucleotide polymerization initiation site in a polymerase-catalyzed primer extension reaction. Alternatively, the 3′ end of the sequencing primer can lack a 3′ OH moiety, or can comprise a terminal 3′ blocking group that inhibits nucleotide polymerization in a polymerase-catalyzed reaction. Any one nucleotide, or more than one nucleotide, along the length of the sequencing primer can be labeled with a detectable reporter moiety. The sequencing primer can be in solution (e.g., a soluble primer).

In some embodiments of the methods described herein, the primer hybridizing steps can be conducted using a hybridization reagent which comprises at least one aqueous solvent, a pH buffering agent, a monovalent cation and a chaotropic agent. In some embodiments, the hybridization reagent optionally comprises any one or any combination of two or more of a detergent, a reducing agent, a chelating agent, an alcohol, a zwitterion, a sugar alcohol and/or a crowding agent. In some embodiments, the aqueous solvent comprises water. In some embodiment, the pH buffering agent comprises NaCl. In some embodiments, the chaotropic agent comprise SDS (sodium dodecyl sulfate), urea, thiourea, guanidinium chloride, guanidine hydrochloride, guanidine thiocyanate, guanidine isothionate, potassium thiocyanate, lithium chloride, sodium iodide or sodium perchlorate.

In some embodiments, the primer hybridizing steps can be conducted at a temperature of about 20-25 °C, or about 25-35 °C, or about 35-45 °C, or about 45-55 °C, or about 55-65 °C, or about 65-75 °C, or higher. In some embodiments, the primer hybridizing can be conducted for about 10-60 seconds, about 1-3 minutes, about 3-6 minutes, about 6-9 minutes, about 9-12 minutes, or longer.

In some embodiments, the primer hybridizing steps can be conducted at several different temperatures using a “step down” temperature regime. For example, a first primer hybridization can be conducted at about 65-45 °C. A second primer hybridization can be conducted at about 25-45 °C. A third primer hybridization can be conducted at about 30-15 °C In some embodiments, each of the primer hybridizing steps in the step down regime can be conducted for the same length of time or different lengths of time. For example, any of the primer hybridizing steps can be conducted for about 10-60 seconds, about 1-3 minutes, about 3-6 minutes, about 6-9 minutes, about 9-12 minutes, or longer.

In some embodiments, the nucleic acid template molecule is a single-stranded nucleic acid template molecule. In some embodiments, the methods comprise sequencing at least one single-stranded nucleic acid template molecule.

In some embodiments of the methods described herein, any portion of the at least one single-stranded nucleic acid template molecule is immobilized to the support or immobilized to a coating on the support. In some embodiments, the 5′ end of the template molecule is immobilized to the support. In some embodiments, the 3′ end of the template molecule is immobilized to the support. In some embodiments, an internal portion of the template molecule is immobilized to the support. In some embodiments, a combination of one or more of the 5′ end, the 3′ end and/or an internal portion of the template molecule is immobilized to the support.

In some embodiments, the 5′ end of the at least one single-stranded nucleic acid template molecule is covalently attached to a surface primer which is immobilized to the support or immobilized to a coating on the support. In some embodiments, the 5′ end of the at least one single-stranded nucleic acid template molecule is hybridized to a surface primer which is immobilized to the support or immobilized to a coating on the support. In some embodiments, in any of the methods for sequencing described herein, the 3′ end of the at least one single-stranded nucleic acid template molecule is covalently attached to a surface primer which is immobilized to the support or immobilized to a coating on the support. In some embodiments, the 3′ end of the at least one single-stranded nucleic acid template molecule is hybridized to a surface primer which is immobilized to the support or immobilized to a coating on the support.

In some embodiments, the at least one single-stranded nucleic acid template molecule can be generated by a clonal amplification workflow. In some embodiments, the at least one single-stranded nucleic acid template molecule was not generated by a clonal amplification workflow.

In some embodiments, the at least one single-stranded nucleic acid template molecule comprises at least one uridine nucleotide. In some embodiments, in any of the methods for sequencing described herein, the at least one single-stranded nucleic acid template molecule does not comprise a uridine nucleotide.

In some embodiments, the at least one single-stranded nucleic acid template molecule comprises one single copy of the sequence-of-interest (“one-copy template molecule”). For example, the one-copy template molecule can be generated by bridge amplification. In some embodiments, the at least one single-stranded nucleic acid template molecule comprises two or more tandem copies of the sequence-of-interest. For example, the single stranded nucleic acid template molecule comprises about 2, 5, 10, 20, 50, 100, 150, 200, 250, 300, 350, 400, 450, 500, 550, 700, 900, 1000, 1500, or 2000 copies of the sequence of interest, or any range therebetween. For example, the tandem-copy template molecule comprises a concatemer which can be generated by rolling circle amplification (RCA).

In some embodiments, the at least one single-stranded nucleic acid template molecule comprises at least one sequence-of-interest (e.g., insert region). In some embodiments, the at least one single-stranded nucleic acid template molecule comprises at least one universal adaptor sequence including any one or any combination of: a first surface primer binding site (e.g., capture primer binding site); a second surface primer binding site (e.g., surface pinning primer binding site); a first sequencing primer binding site (e.g., forward sequencing primer binding site); a second sequencing primer binding site (e.g., reverse sequencing primer binding site); a first amplification primer binding site (e.g., forward amplification primer binding site); a second amplification primer binding site (e.g., reverse amplification primer binding site); a first sample index sequence; a second sample index sequence; a first unique molecular tag sequence; a second unique molecular tag sequence; a first compaction oligonucleotide binding site; and/or a second compaction oligonucleotide binding site.

In some embodiments of the methods described herein, the sequencing read products comprise a sequencing primer (e.g., a nucleic acid oligonucleotide) which is joined to a polynucleotide having a sequence that is complementary to at least a portion of the single-stranded nucleic acid template molecule. The at least a portion of the complementary polynucleotide can be generated by any type of sequencing workflow comprising a polymerase-catalyzed primer extension reaction in which the sequence of the single-strand template molecule is determined. The at least a portion of the complementary polynucleotide can be generated by any type of polymerase-catalyzed primer extension reaction in which the sequence of the single-strand template molecule is not determined. In some embodiments, the sequencing primer portion of the sequencing read product has an extendible 3′ terminal end. In some embodiments, the sequencing primer portion of the sequencing read product has a non-extendible 3′ terminal end. Various methods for polymerase-catalyzed sequencing are described below.

In some embodiments of the methods described herein, the support is a planar support (e.g., a slide). In some embodiments, the support is a non-planar support (e.g., a bead). In some embodiments, the support comprises or consists of a solid or material. In some embodiments, the support comprises or consists of a semi-solid material. In some embodiments, the support comprises or consists of a porous material. In some embodiments, the support comprises or consists of a semi-porous material. In some embodiments, the support comprises or consists of a non-porous material. The support can be made of any suitable material known in the art or described herein, such as glass, plastic or a polymer material.

In some embodiments, the surface of the support can be coated with one or more compounds to produce a passivated layer on the support. In some embodiments, the passivated layer forms a porous layer. In some embodiments, the passivated layer forms a semi-porous layer. In some embodiments, the surface primer or the single-stranded nucleic template molecule can be attached to the passivated layer to immobilize the primer or template molecule to the support. In some embodiments, the support comprises a low non-specific binding surface that enables improved nucleic acid hybridization, amplification and sequencing performance on the support. In general, the support may comprise one or more layers of a covalently or non-covalently attached low-binding, chemical modification layers, e.g., silane layers, polymer films, and one or more covalently or non-covalently attached oligonucleotides that can be used for immobilizing a plurality of nucleic acid template molecules to the support. In some embodiments, the support can comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacet-amidylpentyl)acrylamide-co-acrylamide (PAZAM). In some embodiments, the support comprises a surface coating having at least one hydrophilic polymer coating layer and at least one layer of a plurality of oligonucleotides. The hydrophilic polymer coating layer can comprise polyethylene glycol (PEG). The hydrophilic polymer coating layer can comprise branched PEG having at least 4 branches. In some embodiments, the low non-specific binding coating has a degree of hydrophilicity which can be measured as a water contact angle, where the water contact angle is no more than 45 degrees.

In some embodiments, the support comprises a plurality of nucleic acid template molecules immobilized to the support or immobilized to a coating on the support. In some embodiments, about 10²-10¹⁵ nucleic acid template molecule are immobilized to the support, with nucleic acid template molecules being immobilized to different sites on the support. In some embodiments, the plurality of nucleic acid template molecule are immobilized to pre-determined sites (e.g., locations) on the support. In some embodiments, the plurality of nucleic acid template molecules are immobilized to random sites (e.g., locations) on the support. In some embodiments, the plurality of nucleic acid template molecules immobilized to the support are in fluid communication with each other to permit flowing a solution of reagents (e.g., enzymes including polymerases, multivalent molecules, nucleotides and/or divalent cations, and the like) onto the support, so that the plurality of nucleic acid template molecules on the support can be reacted with the solution of reagents in a massively parallel manner. In some embodiments, the nucleic acid template molecules are single-stranded.

In some embodiments of the methods described herein, the at least one nucleic acid template molecule comprises a sequence-of-interest (e.g., insert region) flanked on both sides by at least one universal adaptor sequence. An illustrative nucleic acid template molecule may comprise (ordered from 3′ to 5′; see FIG. 1): a first surface primer binding site (e.g., capture primer binding site); a first sample index sequence; a second sequencing primer binding site (e.g., reverse sequencing primer binding site); a sequence-of-interest; a first sequencing primer binding site (e.g., forward sequencing primer binding site); a second sample index sequence; and a second surface primer binding site (e.g., surface pinning primer binding site). The skilled artisan appreciates that many other arrangements of the sequence-of-interest and universal adaptor sequences are possible. The illustrative template molecule can be a repeating unit of a concatemer template molecule having two or more tandem copies of the sequence-of-interest, or the illustrative template molecule can be a single copy template molecule having one copy of the sequence-of-interest.

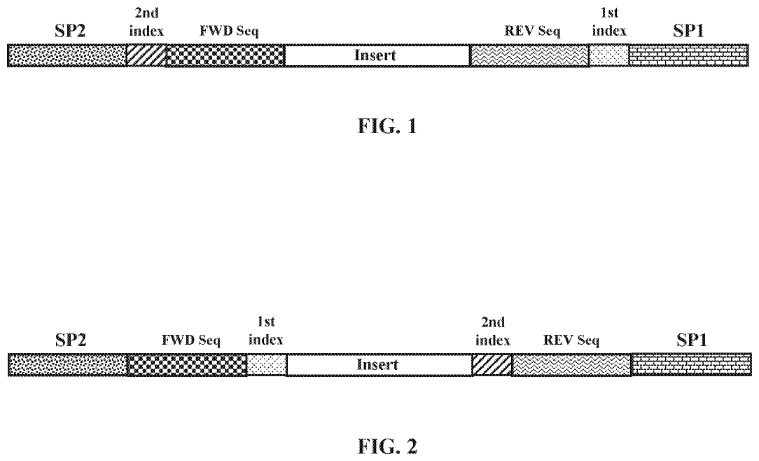

In some embodiments, a sequencing primer (e.g., first, second, third or fourth sequencing primer) that is employed to sequence the sequence-of-interest region hybridizes to the first sequencing primer binding site (e.g., forward sequencing primer binding site). See for example FIG. 1 or 2.

In some embodiments, a sequencing primer (e.g., first, second, third or fourth sequencing primer) that is employed to sequence the first sample index region hybridizes to a portion of the second sequencing primer binding site (e.g., reverse sequencing primer binding site). See for example FIG. 1.

In some embodiments, a sequencing primer (e.g., first, second, third or fourth sequencing primer) that is employed to sequence the first sample index region hybridizes to a portion of the first sequencing primer binding site (e.g., forward sequencing primer binding site). See for example FIG. 2.

In some embodiments, a sequencing primer (e.g., first, second, third or fourth sequencing primer) that is employed to sequence the second sample index region hybridizes to a portion of the second surface primer binding site (e.g., surface pinning primer binding site). See for example FIG. 1.

In some embodiments, a sequencing primer (e.g., first, second, third or fourth sequencing primer) that is employed to sequence the second sample index region hybridizes to a portion of the second sequencing primer binding site (e.g., reverse sequencing primer binding site). See for example FIG. 2.

In some embodiments, the order of sequencing the nucleic acid template molecule is as follows: Read 1 (wherein the full length of the sequence-of-interest is sequenced); followed by the first sample index. In some embodiments, the second sample index is not sequenced. In some embodiments, the second index is sequenced.

In some embodiments, the order of sequencing the nucleic acid template molecule is as follows: Read 1 (wherein the full length of the sequence-of-interest is sequenced); followed by the first sample index; followed by the second sample index (e.g., FIG. 10).

In some embodiments, the order of sequencing the nucleic acid template molecule is as follows: Read 1 (wherein only a portion of the sequence-of-interest; e.g., 5-25 nucleotides are sequenced), followed by the first sample index, followed by the second sample index. In some embodiments, sequencing the second sample index is omitted.

In some embodiments, the order of sequencing the nucleic acid template molecule is as follows: Read 1 (wherein only a portion of the sequence-of-interest; e.g., 5-25 nucleotides is sequenced), followed by the first sample index, followed by the second sample index, followed by Read 1 (wherein the full length of the sequence-of-interest is sequenced) (see e.g., FIG. 11). In some embodiments, sequencing the second sample index is omitted.

In some embodiments, the order of sequencing the nucleic acid template molecule is as follows: first sample index; followed by Read 1 (wherein the full length of the sequence-of-interest is sequenced).

In some embodiments, the order of sequencing the nucleic acid template molecule is as follows: first sample index; followed by Read 1 (wherein only a portion of the sequence-of-interest; e.g., 5-25 nucleotides, is sequenced).

In some embodiments, the order of sequencing the nucleic acid template molecule is as follows: first sample index; followed by the second sample index; followed by Read 1 (wherein the full length of the sequence-of-interest is sequenced) (see e.g., FIG. 12).

In some embodiments, the order of sequencing the nucleic acid template molecule is as follows: first sample index; followed by the second sample index; followed by Read 1 (wherein only a portion of the sequence-of-interest; e.g., 5-25 nucleotides, is sequenced).

In some embodiments, the order of sequencing the nucleic acid template molecule is as follows: first sample index; followed by Read 1 (wherein the full length of the sequence-of-interest is sequenced); followed by the second sample index.

In some embodiments, the order of sequencing the nucleic acid template molecule is as follows: first sample index; followed by Read 1 (wherein only a portion of the sequence-of-interest; e.g., 5-25 nucleotides is sequenced); followed by the second sample index; and optionally followed by Read 1 (wherein the full length the sequence-of-interest is sequenced) (see e.g., FIG. 13).

The skilled artisan will recognize that the sequence-of-interest, first sample index and second sample index can be sequenced in any order. The skilled artisan will recognize that the full length or only a portion of the sequence-of-interest, first sample index and second sample index can be sequenced in any order.

In some embodiments, the nucleic acid template molecule can be sequenced by employing a pairwise sequencing workflow which includes sequencing the first template strand, conducting a primer extension reaction for a second strand synthesis step (e.g., sometimes called a “pairwise turn”), and sequencing a template strand having a sequence that is complementary to the first template strand.

In some embodiments of the pairwise sequencing workflow, the order of sequencing the nucleic acid template molecule is as follows: first sample index; followed by the second sample index; followed by Read 1 (wherein the full length of the sequence-of-interest is sequenced); followed by second strand synthesis; followed by Read 2 (wherein the full length of the sequence-of-interest is sequenced) (see, e.g., FIG. 14).

In some embodiments of the pairwise sequencing workflow, the order of sequencing the nucleic acid template molecule is as follows: Read 1 (wherein the full length the sequence-of-interest is sequenced); followed by second strand synthesis (pairwise turn); followed by Read 2 (wherein the full length the sequence-of-interest is sequenced); followed by the first sample index; followed by the second sample index.

In some embodiments, in a pairwise sequencing workflow, the order of sequencing the nucleic acid template molecule is as follows: Read 1 (wherein the full length the sequence-of-interest is sequenced); followed by second strand synthesis (pairwise turn); followed by the first sample index; followed by the second sample index; followed by Read 2 (wherein the full length of the sequence-of-interest is sequenced) (see, e.g., FIG. 15).

The skilled artisan will recognize that the sequence-of-interest, first sample index and second sample index can be sequenced in any order. The skilled artisan will recognize that the full length or only a portion of the sequence-of-interest, first sample index and second sample index can be sequenced in any order.

### Methods for Sequencing

The present disclosure provides methods for sequencing any of the nucleic acid template molecules described herein. In some embodiments, the nucleic acid template molecules are concatemer template molecules. Any of the methods for conducting rolling circle amplification reaction described herein can be used to generate a plurality of concatemer template molecules immobilized to a support (“immobilized concatemer molecules”), and the immobilized concatemer molecules can be subjected to sequencing reactions. In some embodiments, the sequencing reactions employ detectably labeled nucleotide analogs. In some embodiments, the sequencing reactions employ a two-stage sequencing reaction comprising binding detectably labeled multivalent molecules, and incorporating nucleotide analogs. In some embodiments, the sequencing reactions employ non-labeled nucleotide analogs.

In some embodiments, any of the rolling circle amplification reaction described herein (e.g., RCA conducted on-support or in-solution) can be used to generate immobilized concatemer molecules each containing tandem repeat units of the sequence-of-interest and any adaptor sequences present in the covalently closed circular library molecules. Methods for generating circular library molecules are described, for example in WO2023168444 and WO2023168443, the contents of which are incorporated by reference in their entireties.

For example, the tandem repeat unit may comprise: (i) a first left universal adaptor sequence having a binding sequence for a first surface primer, (ii) a second left universal adaptor sequence having a binding sequence for a first sequencing primer, (iii) a sequence-of-interest, (iv) a second right universal adaptor sequence having a binding sequence for a second sequencing primer, (v) a first right universal adaptor sequence having a binding sequence for a second surface primer, and (vii) a first left index sequence and/or a first right index sequence (e.g., see FIGS. 1 and 2). In some embodiments, the tandem repeat unit further comprises a first left unique identification sequence and/or a first right unique identification sequence.

The immobilized concatemer molecule can self-collapse into a compact nucleic acid nanoball. Inclusion of one or more compaction oligonucleotides during the RCA reaction can further compact the size and/or shape of the nanoball. An increase in the number of tandem repeat units in a given concatemer increases the number of sites along the concatemer molecule for hybridizing to multiple sequencing primers (e.g., sequencing primers having a universal sequence) which serve as multiple initiation sites for polymerase-catalyzed sequencing reactions. When the sequencing reaction employs detectably labeled nucleotides and/or detectably labeled multivalent molecules (e.g., having nucleotide units), the signals emitted by the nucleotides or nucleotide units that participate in the parallel sequencing reactions along the concatemer molecules yield an increased signal intensity for each concatemer molecule. Multiple portions of a given concatemer molecule can be simultaneously sequenced. Furthermore, a plurality of binding complexes can form along a concatemer molecule, each binding complex comprising a sequencing polymerase bound to a multivalent molecule, wherein the plurality of binding complexes remain stable without dissociation, resulting in increased persistence time, which increases signal intensity and reduces imaging time.

### Methods for Sequencing Using Nucleotide Analogs

The present disclosure provides methods for sequencing any of the nucleic acid template molecules described herein, the methods comprising step (a): contacting a sequencing polymerase with (i) a nucleic acid template molecule and (ii) a nucleic acid sequencing primer, wherein the contacting is conducted under conditions suitable to bind the sequencing polymerase to the nucleic acid template molecule, which is hybridized to the nucleic acid primer. The nucleic acid template molecule hybridized to the nucleic acid primer can form a nucleic acid duplex. In some embodiments, the sequencing polymerase comprises a recombinant mutant sequencing polymerase that can incorporate nucleotide analogs. In some embodiments, the sequencing primer comprises a 3′ extendible end. In some embodiments, the nucleic acid template molecule is immobilized.

In some embodiments, the sequencing primer comprises a 3′ extendible end. In some embodiments, the sequencing primer comprises a 3′ non-extendible end. In some embodiments, the plurality of nucleic acid template molecules comprises amplified template molecules (e.g., clonally amplified template molecules). In some embodiments, individual nucleic acid template molecules in the plurality of nucleic acid template molecules comprise one copy of a target sequence of interest. In some embodiments, individual template molecules in the plurality of nucleic acid molecules comprise two or more tandem copies of a target sequence of interest (e.g., concatemer template molecules). In some embodiments, the nucleic acid template molecules in the plurality of nucleic acid template molecules comprise the same target sequence of interest. In some embodiments, nucleic acid template molecules in the plurality of nucleic acid template molecules comprises a different target sequences of interest. In some embodiments, the plurality of nucleic acid template molecules and/or the plurality of nucleic acid primers is in solution. In some embodiments, the plurality of nucleic acid template molecules and/or the plurality of nucleic acid primers is immobilized to a support. In some embodiments, when the plurality of nucleic acid template molecules and/or the plurality of nucleic acid primers are immobilized to a support, the binding of the plurality of first sequencing polymerases generates a plurality of immobilized first complexed polymerases. In some embodiments, the plurality of nucleic acid template molecules and/or the plurality of nucleic acid primers are immobilized to 10²-10¹⁵ different sites on a support. In some embodiments, the binding of the plurality of nucleic acid template molecules and the plurality of nucleic acid primers with the plurality of first sequencing polymerases generates a plurality of first complexed polymerases immobilized to 10²-10¹⁵ different sites on the support. In some embodiments, the plurality of immobilized first complexed polymerases on the support are immobilized to pre-determined sites on the support. In some embodiments, the plurality of immobilized first complexed polymerases on the support are immobilized to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some embodiments, the methods for sequencing further comprise step (b): contacting the sequencing polymerase bound to the nucleic acid duplex with a plurality of nucleotides under conditions suitable for binding at least one nucleotide to the sequencing polymerase and suitable for polymerase-catalyzed nucleotide incorporation. In some embodiments, the sequencing polymerase is contacted with the plurality of nucleotides in the presence of at least one catalytic cation comprising magnesium and/or manganese. In some embodiments, the plurality of nucleotides comprises at least one nucleotide analog having a chain terminating moiety at the sugar 2′ or 3′ position. In some embodiments, the chain terminating moiety is removable from the sugar 2′ or 3′ position to convert the chain terminating moiety to an OH or H group. In some embodiments, the plurality of nucleotides comprises at least one nucleotide that lacks a chain terminating moiety. In some embodiments, at least on nucleotide is labeled with a detectable reporter moiety (e.g., fluorophore). In some embodiments, step (b) further comprises removing the chain terminating moiety from the incorporated chain terminating nucleotide to generate an extendible 3′OH group. In some embodiments, step (b) comprises repeating at least once the steps of: (i) incorporating a detectably labeled chain terminating nucleotide into the terminal 3′ end of a hybridized first sequencing primer; (ii) detecting and identifying the incorporated chain terminating nucleotide; and (iii) removing the chain terminating moiety and/or the detectable label from the incorporated chain terminating nucleotide to generate an extendible 3′OH sugar group on the incorporated chain terminating nucleotide.

In some embodiments, the methods for sequencing comprise step (c): incorporating at least one nucleotide into the 3′ ends of the extendible primers under conditions suitable for incorporating the at least one nucleotide. In some embodiments, the suitable conditions for nucleotide binding the polymerase and for incorporation of the nucleotide into the primer extension product can be the same or different. In some embodiments, conditions suitable for incorporating the nucleotide comprise inclusion of at least one catalytic cation comprising magnesium and/or manganese. In some embodiments, the at least one nucleotide binds the sequencing polymerase and incorporates into the 3′ end of the extendible primer. In some embodiments, the incorporating the nucleotide into the 3′ end of the primer in step (c) comprises a primer extension reaction.

In some embodiments, the methods for sequencing comprise step (d): repeating the incorporating at least one nucleotide into the 3′ end of the extendible primer of steps (b) and (c) at least once. In some embodiments, the plurality of nucleotides comprises a plurality of nucleotides labeled with detectable reporter moiety. The detectable reporter moiety can comprise a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to identity of the nucleotide base (e.g., all of dATP, dGTP, dCTP, dTTP or dUTP are labeled with the same fluorophore) to permit detection and identification of the nucleotide base. In some embodiments, the method comprises detecting the at least one incorporated nucleotide at step (c) and/or (d). In some embodiments, the method comprises identifying the at least one incorporated nucleotide at step (c) and/or (d). In some embodiments, the sequence of the nucleic acid template molecule can be determined by detecting and identifying the nucleotide that binds the sequencing polymerase, thereby determining the sequence of the nucleic acid template molecule. In some embodiments, the sequence of the nucleic acid template molecule can be determined by detecting and identifying the nucleotide that incorporates into the 3′ end of the primer, thereby determining the sequence of the template molecule.

In some embodiments, the plurality of sequencing polymerases that are bound to the nucleic acid duplexes comprise a plurality of complexed polymerases, having at least a first and second complexed polymerase, wherein (a) the first complexed polymerases comprises a first sequencing polymerase bound to a first nucleic acid duplex comprising a first nucleic acid template sequence which is hybridized to a first nucleic acid primer, (b) the second complexed polymerases comprises a second sequencing polymerase bound to a second nucleic acid duplex comprising a second nucleic acid template sequence which is hybridized to a second nucleic acid primer, (c) the first and second nucleic acid template sequences comprise the same or different sequences, (d) the first and second nucleic acid template are clonally-amplified, (e) the first and second primers comprise extendible 3′ ends or non-extendible 3′ ends, and (f) the plurality of complexed polymerases are immobilized to a support. In some embodiments, the density of the plurality of complexed polymerases is about 10²-10¹⁵ complexed polymerases per mm² that are immobilized to the support.

### Two-Stage Methods for Nucleic Acid Sequencing

The present disclosure provides a two-stage method for sequencing any of the nucleic acid template molecules described herein. The first stage generally comprises binding multivalent molecules to complexed polymerases to form multivalent-complexed polymerases, and detecting the multivalent-complexed polymerases. In some embodiments, the nucleic acid template molecules are immobilized (“immobilized nucleic acid template molecules”).

In some embodiments, the first stage comprises step (a): contacting a plurality of first sequencing polymerases to (i) a plurality of nucleic acid template molecules and (ii) a plurality of nucleic acid sequencing primers, wherein the contacting is conducted under conditions suitable to bind the plurality of first sequencing polymerases to the plurality of nucleic acid template molecules and the plurality of nucleic acid primers thereby forming a plurality of first complexed polymerases each comprising a first sequencing polymerase bound to a nucleic acid duplex, wherein the nucleic acid duplex comprises a nucleic acid template molecule hybridized to a nucleic acid primer. In some embodiments, the first sequencing polymerase comprises a recombinant mutant sequencing polymerase. In some embodiments, the sequencing primer comprises a 3′ extendible end.

In some embodiments, the sequencing primer comprises a 3′ extendible end. In some embodiments, the sequencing primer comprises a 3′ non-extendible end. In some embodiments, the plurality of nucleic acid template molecules comprise amplified template molecules (e.g., clonally amplified template molecules). In some embodiments, the plurality of nucleic acid template molecules comprise one copy of a target sequence of interest. In some embodiments, the plurality of nucleic acid molecules comprise two or more tandem copies of a target sequence of interest (e.g., concatemer template molecules). In some embodiments, the nucleic acid template molecules in the plurality of nucleic acid template molecules comprise the same target sequence of interest or different target sequences of interest. In some embodiments, the plurality of nucleic acid template molecules and/or the plurality of nucleic acid primers are in solution or are immobilized to a support. In some embodiments, when the plurality of nucleic acid template molecules and/or the plurality of nucleic acid primers are immobilized to a support, the binding with the first sequencing polymerase generates a plurality of immobilized first complexed polymerases. In some embodiments, the plurality of nucleic acid template molecules and/or nucleic acid primers are immobilized to 10²-10¹⁵ different sites on a support. In some embodiments, the binding of the plurality of nucleic acid template molecules and nucleic acid primers with the plurality of first sequencing polymerases generates a plurality of first complexed polymerases immobilized to 10²-10¹⁵ different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some embodiments, the methods for sequencing comprise step (b): contacting the plurality of first complexed polymerases with a plurality of multivalent molecules to form a plurality of multivalent-complexed polymerases (e.g., binding complexes). In some embodiments, individual multivalent molecules in the plurality of multivalent molecules comprise a core attached to multiple nucleotide arms and individual nucleotide arms are attached to a nucleotide (e.g., nucleotide unit) (e.g., FIGS. 19-23). In some embodiments, the contacting of step (b) is conducted under a condition suitable for binding complementary nucleotide units of the multivalent molecules to at least two of the plurality of first complexed polymerases thereby forming a plurality of multivalent-complexed polymerases. In some embodiments, the condition is suitable for inhibiting polymerase-catalyzed incorporation of the complementary nucleotide units into the primers of the plurality of multivalent-complexed polymerases. In some embodiments, the plurality of multivalent molecules comprise at least one multivalent molecule having multiple nucleotide arms (e.g., FIGS. 19-23) each attached with a nucleotide analog (e.g., nucleotide analog unit), where the nucleotide analog includes a chain terminating moiety at the sugar 2′ and/or 3′ position. In some embodiments, the plurality of multivalent molecules comprises at least one multivalent molecule comprising multiple nucleotide arms each attached with a nucleotide unit that lacks a chain terminating moiety. In some embodiments, at least one of the multivalent molecules in the plurality of multivalent molecules is labeled with a detectable reporter moiety. In some embodiments, the detectable reporter moiety comprises a fluorophore. In some embodiments, the contacting of step (b) is conducted in the presence of at least one non-catalytic cation comprising strontium, barium and/or calcium.

In some embodiments, the methods for sequencing comprise step (c): detecting the plurality of multivalent-complexed polymerases. In some embodiments, the detecting comprises detecting the multivalent molecules that are bound to the complexed polymerases, where the complementary nucleotide units of the multivalent molecules are bound to the primers but incorporation of the complementary nucleotide units is inhibited. In some embodiments, the multivalent molecules are labeled with a detectable reporter moiety to permit detection. In some embodiments, the labeled multivalent molecules comprise a fluorophore attached to the core, linker and/or nucleotide unit of the multivalent molecules.

In some embodiments, the methods for sequencing comprise step (d): identifying the nucleobase of the complementary nucleotide units that are bound to the plurality of first complexed polymerases, thereby determining the sequence of the template molecule. In some embodiments, the multivalent molecules are labeled with a detectable reporter moiety that corresponds to the particular nucleotide units attached to the nucleotide arms to permit identification of the complementary nucleotide units (e.g., nucleotide base adenine, guanine, cytosine, thymine or uracil) that are bound to the plurality of first complexed polymerases.

In some embodiments, the second stage of the two-stage sequencing method generally comprises nucleotide incorporation. In some embodiments, the methods for sequencing comprises step (e): dissociating the plurality of multivalent-complexed polymerases and removing the plurality of first sequencing polymerases and their bound multivalent molecules, and retaining the plurality of nucleic acid duplexes.

In some embodiments, the methods for sequencing comprises step (f): contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a nucleic acid duplex. In some embodiments, the second sequencing polymerase comprises a recombinant mutant sequencing polymerase.

In some embodiments, the plurality of first sequencing polymerases of step (a) comprise an amino acid sequence that is 100% identical to the amino acid sequence as the plurality of the second sequencing polymerases of step (f). In some embodiments, the plurality of first sequencing polymerases of step (a) comprise an amino acid sequence that differs from the amino acid sequence of the plurality of the second sequencing polymerases of step (f).

In some embodiments, the methods for sequencing comprise step (g): contacting the plurality of second complexed polymerases with a plurality of nucleotides, wherein the contacting is conducted under conditions suitable for binding complementary nucleotides from the plurality of nucleotides to at least two of the second complexed polymerases thereby forming a plurality of nucleotide-complexed polymerases. In some embodiments, the contacting of step (g) is conducted under conditions that are suitable for promoting polymerase-catalyzed incorporation of the bound complementary nucleotides into the primers of the nucleotide-complexed polymerases, thereby forming a plurality of nucleotide-complexed polymerases. In some embodiments, the incorporating the nucleotide into the 3′ end of the primer in step (g) comprises a primer extension reaction. In some embodiments, the contacting of step (g) is conducted in the presence of at least one catalytic cation comprising magnesium and/or manganese. In some embodiments, the plurality of nucleotides comprise native nucleotides (e.g., non-analog nucleotides) or nucleotide analogs. In some embodiments, the plurality of nucleotides comprise a 2′ and/or 3′ chain terminating moiety which is removable or is not removable. In some embodiments, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base or is not cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base. In some embodiments, the plurality of nucleotides are non-labeled nucleotides.

In some embodiments, the methods for sequencing comprise step (h): detecting the complementary nucleotides which are incorporated into the primers of the nucleotide-complexed polymerases. In some embodiments, the plurality of nucleotides are labeled with a detectable reporter moiety to permit detection. In some embodiments, the detecting of step (h) is omitted. In some embodiments, when a plurality of non-labeled nucleotide are employed for step (g), then the detecting of step (h) is omitted.

In some embodiments, the methods for sequencing comprise step (i): identifying the bases of the complementary nucleotides which are incorporated into the primers of the nucleotide-complexed polymerases. In some embodiments, the identification of the incorporated complementary nucleotides in step (i) can be used to confirm the identity of the complementary nucleotides of the multivalent molecules that are bound to the plurality of first complexed polymerases in step (d). In some embodiments, the identifying of step (i) can be used to determine the sequence of the nucleic acid template molecules. In some embodiments, the identifying of step (i) is omitted. In some embodiments, when a plurality of non-labeled nucleotide are employed for step (g), then the identifying of step (i) is omitted.

In some embodiments, the methods for sequencing comprise step (j): removing the chain terminating moiety from the incorporated nucleotide when step (g) is conducted by contacting the plurality of second complexed polymerases with a plurality of nucleotides that comprise at least one nucleotide having a 2′ and/or 3′ chain terminating moiety.

In some embodiments, the methods for sequencing comprise step (k): repeating steps (a)-(j) at least once. In some embodiments, the sequence of the nucleic acid template molecules can be determined by detecting and identifying the multivalent molecules that bind the sequencing polymerases but do not incorporate into the 3′ end of the primer at steps (c) and (d). In some embodiments, the sequence of the nucleic acid template molecule can be determined (or confirmed) by detecting and identifying the nucleotide that incorporates into the 3′ end of the primer at steps (h) and (i).

In some embodiments, the method comprise sequencing nucleic acid concatemer template molecules.

In some embodiments of the methods for sequencing nucleic acid concatemer template molecules described herein, the binding of the plurality of first complexed polymerases with the plurality of multivalent molecules forms at least one avidity complex. In some embodiments, the method comprises the steps: (a) binding a first nucleic acid primer, a first sequencing polymerase, and a first multivalent molecule to a first portion of a concatemer template molecule thereby forming a first binding complex, wherein a first nucleotide unit of the first multivalent molecule binds to the first sequencing polymerase; and (b) binding a second nucleic acid primer, a second sequencing polymerase, and the first multivalent molecule to a second portion of the same concatemer template molecule, thereby forming a second binding complex, wherein a second nucleotide unit of the first multivalent molecule binds to the second sequencing polymerase, wherein the first and second binding complexes which comprise the same multivalent molecule forms an avidity complex. In some embodiments, the first sequencing polymerase comprises any wild type or mutant polymerase described herein. In some embodiments, the second sequencing polymerase comprises any wild type or mutant polymerase described herein. The concatemer template molecule comprises tandem repeat sequences of a sequence of interest and at least one universal sequencing primer binding site. The first and second nucleic acid primers can bind to a sequencing primer binding site along the concatemer template molecule. Exemplary multivalent molecules are shown in FIGS. 19-23.

In some embodiments of the methods for sequencing nucleic acid concatemer template molecules, the method comprises binding the plurality of first complexed polymerases with the plurality of multivalent molecules to form at least one avidity complex. In some embodiments, the method comprises the steps: (a) contacting the plurality of sequencing polymerases and the plurality of nucleic acid primers with different portions of a nucleic acid concatemer template molecule to form at least first and second complexed polymerases on the same concatemer template molecule; (b) contacting a plurality of multivalent molecules with the at least first and second complexed polymerases on the same nucleic acid concatemer template molecule, under conditions suitable to bind a single multivalent molecule from the plurality to the first and second complexed polymerases, wherein at least a first nucleotide unit of the single multivalent molecule is bound to the first complexed polymerase which comprises a first primer hybridized to a first portion of the nucleic acid concatemer template molecule, thereby forming a first binding complex (e.g., first ternary complex), and wherein at least a second nucleotide unit of the single multivalent molecule is bound to the second complexed polymerase which comprises a second primer hybridized to a second portion of the nucleic acid concatemer template molecule, thereby forming a second binding complex (e.g., second ternary complex), wherein the contacting is conducted under conditions suitable to inhibit polymerase-catalyzed incorporation of the bound first and second nucleotide units in the first and second binding complexes, and wherein the first and second binding complexes which are bound to the same multivalent molecule forms an avidity complex; (c) detecting the first and second binding complexes on the same nucleic acid concatemer template molecule, and (d) identifying the first nucleotide unit in the first binding complex, thereby determining the sequence of the first portion of the nucleic acid concatemer template molecule, and identifying the second nucleotide unit in the second binding complex, thereby determining the sequence of the second portion of the nucleic acid concatemer template molecule. In some embodiments, the plurality of sequencing polymerases comprise any wild type or mutant sequencing polymerase described herein. The nucleic acid concatemer template molecule comprises tandem repeat sequences of a sequence of interest and at least one universal sequencing primer binding site. The plurality of nucleic acid primers can bind to a sequencing primer binding site along the nucleic acid concatemer template molecule. Exemplary multivalent molecules are shown in FIGS. 19-23.

### Sequencing-by-Binding

The present disclosure provides methods for sequencing any of the immobilized nucleic acid template molecules described herein, wherein the sequencing methods comprise a sequencing-by-binding (SBB) procedure which employs non-labeled chain-terminating nucleotides. In some embodiments, the sequencing-by-binding (SBB) method comprises the steps of: (a) sequentially contacting a primed template nucleic acid with at least two separate mixtures under ternary complex stabilizing conditions, wherein the at least two separate mixtures each comprise a polymerase and a nucleotide, whereby the sequentially contacting results in the primed template nucleic acid being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed template nucleic acid molecule, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed template nucleic acid after step (b), thereby producing an extended primer; and (e) repeating steps (a) through (d) at least once on the primed template nucleic acid that comprises the extended primer. Exemplary sequencing-by-binding methods are described in U.S. Pat. Nos. 10,246,744 and 10,731,141 (where the contents of both patents are hereby incorporated by reference in their entireties).

### Sequencing Polymerases

The present disclosure provides methods for sequencing nucleic acid template molecules, where any of the sequencing methods described herein employ at least one type of sequencing polymerase and a plurality of nucleotides, or employ at least one type of sequencing polymerase and a plurality of nucleotides and a plurality of multivalent molecules. In some embodiments, the sequencing polymerase(s) is/are capable of incorporating a complementary nucleotide opposite a nucleotide in a template molecule. In some embodiments, the sequencing polymerase(s) is/are capable of binding a complementary nucleotide unit of a multivalent molecule opposite a nucleotide in a template molecule. In some embodiments, the plurality of sequencing polymerases comprise recombinant mutant polymerases.

Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT®, DEEP VENT®, THERMINATOR™, Pfu, KOD, Pfx, Tgo and RB69 polymerases. Exemplary polymerases are described, for example in U.S. Pat. No. 11,859,241, the contents of which are incorporated by reference in their entirety herein.

### Nucleotides

The present disclosure provides methods for sequencing nucleic acid molecules, where any of the sequencing methods described herein employ at least one nucleotide. The nucleotides comprise a base, sugar and at least one phosphate group. In some embodiments, at least one nucleotide in the plurality comprises an aromatic base, a five carbon sugar (e.g., ribose or deoxyribose), and one or more phosphate groups (e.g., 1-10 phosphate groups). The plurality of nucleotides can comprise at least one type of nucleotide selected from a group consisting of dATP, dGTP, dCTP, dTTP and dUTP. The plurality of nucleotides can comprise at a mixture of any combination of two or more types of nucleotides selected from a group consisting of dATP, dGTP, dCTP, dTTP and/or dUTP. In some embodiments, at least one nucleotide in the plurality is not a nucleotide analog. In some embodiments, at least one nucleotide in the plurality comprises a nucleotide analog.

In some embodiments, at least one nucleotide in the plurality of nucleotides comprise a chain of one, two or three phosphorus atoms where the chain is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain comprise substituted side groups such as O, S or BH₃. In some embodiments, the chain comprises phosphate groups substituted with analogs such as phosphoramidate, phosphorothioate, phosphordithioate, and O-methylphosphoroamidite groups.

In some embodiments, at least one nucleotide in the plurality of nucleotides comprises a terminator nucleotide analog having a chain terminating moiety (e.g., blocking moiety) at the sugar 2′ position. In some embodiments, at least one nucleotide in the plurality of nucleotides comprises a terminator nucleotide analog having a chain terminating moiety (e.g., blocking moiety) at the sugar 3′ position. In some embodiments, at least one nucleotide in the plurality of nucleotides comprises a terminator nucleotide analog having a chain terminating moiety (e.g., blocking moiety) at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety (e.g., blocking moiety) can be removed to convert the terminator nucleotide into a nucleotide that permits incorporation of a subsequent nucleotide in a polymerase-catalyzed reaction. In some embodiments, the chain terminating moiety is attached to the 3′ sugar position where the sugar comprises a ribose or deoxyribose sugar moiety. In some embodiments, the chain terminating moiety is removable or cleavable from the 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable or removable from the nucleotide, for example by reacting the chain terminating moiety with a chemical agent, by a pH change, by light or by heat. In some embodiments, the chain terminating moieties comprising alkyl, alkenyl, alkynyl or allyl are cleavable with tetrakis(triphenylphosphine)palladium(0) (Pd(PPh₃)₄), with piperidine, or with 2,3-Dichloro-5,6-dicyano-1,4-benzo-quinone (DDQ). In some embodiments, the chain terminating moieties comprising aryl or benzyl are cleavable with H2 Pd/C. In some embodiments, the chain terminating moieties comprising amine, amide, keto, isocyanate, phosphate, thio, or disulfide are cleavable with phosphine or with a thiol group such as beta-mercaptoethanol or dithiothritol (DTT). In some embodiments, the chain terminating moieties comprising carbonate are cleavable with potassium carbonate (K₂CO₃) in MeOH, with triethylamine in pyridine, or with Zn in acetic acid (AcOH). In some embodiments, the chain terminating moieties comprising urea or silyl are cleavable with tetrabutylammonium fluoride, with pyridine-HF, with ammonium fluoride, or with triethylamine trihydrofluoride. In some embodiments, the chain terminating moiety may be cleavable or removable with nitrous acid. In some embodiments, a chain terminating moiety may be cleavable or removable using a solution comprising nitrite, such as, for example, a combination of nitrite with an acid such as acetic acid, sulfuric acid, or nitric acid. In some further embodiments, said solution may comprise an organic acid.

In some embodiments, at least one nucleotide in the plurality of nucleotides comprises a terminator nucleotide analog having a chain terminating moiety (e.g., blocking moiety) at the sugar 2′ position. In some embodiments, at least one nucleotide in the plurality of nucleotides comprises a terminator nucleotide analog having a chain terminating moiety (e.g., blocking moiety) at the sugar 3′ position. In some embodiments, at least one nucleotide in the plurality of nucleotides comprises a terminator nucleotide analog having a chain terminating moiety (e.g., blocking moiety) at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties comprising an azide, azido and/or azidomethyl group are cleavable or removable with a cleaving agent such as a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP). In some embodiments, the chain terminating moiety comprising a 3′-O-amino group or a derivative thereof may be cleaved with nitrous acid, through a mechanism utilizing nitrous acid, or using a solution comprising nitrous acid. In some embodiments, the chain terminating moiety comprising a 3′-O-aminomethyl group or a derivative thereof may be cleaved with nitrous acid, through a mechanism utilizing nitrous acid, or using a solution comprising nitrous acid. In some embodiments, the chain terminating moiety comprising a 3′-O-methylamino group, or a derivative thereof may be cleaved with nitrous acid, through a mechanism utilizing nitrous acid, or using a solution comprising nitrous acid. In some embodiments, the chain terminating moiety comprising a 3′-O-amino group or a derivative thereof may be cleaved using a solution comprising nitrite. In some embodiments, the chain terminating moiety comprising a 3′-O-aminomethyl group or a derivative thereof may be cleaved using a solution comprising nitrite. In some embodiments, the chain terminating moiety comprising 3′-O-methylamino group or a derivative thereof may be cleaved using a solution comprising nitrite. In some embodiments, nitrite may be combined with an acid such as acetic acid, sulfuric acid, or nitric acid. In further embodiments, nitrite may be combined with an organic acid such as, for example, formic acid, acetic acid, propionic acid, butyric acid, isobutyric acid, or the like.

In some embodiments, the nucleotide comprises a chain terminating moiety which is selected from a group consisting of 3′-deoxy nucleotides, 2′,3′-dideoxynucleotides, or a nucleotide that comprises one of the following 3′ modifications: 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the plurality of nucleotides comprises a plurality of nucleotides labeled with detectable reporter moiety. The detectable reporter moiety may comprise a fluorophore. In some embodiments, the fluorophore is attached to the nucleotide base. In some embodiments, the fluorophore is attached to the nucleotide base with a linker which is cleavable or removable from the base. In some embodiments, at least one of the nucleotides in the plurality of nucleotides is not labeled with a detectable reporter moiety. In some embodiments, the plurality of nucleotides comprises nucleotides labeled with a detectable reporter moiety, and nucleotides that are not labeled with a detectable reporter moiety. In some embodiments, a particular detectable reporter moiety (e.g., a fluorophore) that is attached to the nucleotide can correspond to the nucleotide base (e.g., dATP, dGTP, dCTP, dTTP or dUTP) to permit detection and identification of the nucleotide base.

In some embodiments, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the cleavable linker on the base is cleavable or removable from the base by reacting the cleavable moiety with a chemical agent, by a pH change, by light or by heat. In some embodiments, the cleavable moieties comprising alkyl, alkenyl, alkynyl or allyl are cleavable with tetrakis(triphenylphosphine)palladium(0) (Pd(PPh₃)₄), with piperidine, or with 2,3-Dichloro-5,6-dicyano-1,4-benzo-quinone (DDQ). In some embodiments, the cleavable moieties comprising aryl or benzyl are cleavable with H2 Pd/C. In some embodiments, the cleavable moieties comprising amine, amide, keto, isocyanate, phosphate, thio, or disulfide are cleavable with phosphine or with a thiol group such as beta-mercaptoethanol or dithiothritol (DTT). In some embodiments, the cleavable moieties comprising carbonate are cleavable with potassium carbonate (K₂CO₃) in MeOH, with triethylamine in pyridine, or with Zn in acetic acid (AcOH). In some embodiments, the cleavable moieties comprising urea or silyl are cleavable with tetrabutylammonium fluoride, with pyridine-HF, with ammonium fluoride, or with triethylamine trihydrofluoride.

In some embodiments, the cleavable linker on the nucleotide base comprises a cleavable moiety comprising an azide, azido or azidomethyl group. In some embodiments, the cleavable moieties comprising azide, azido or azidomethyl group are cleavable or removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the chain terminating moiety (e.g., the chain terminating moiety located at the sugar 2′ and/or sugar 3′ position) and the cleavable linker on the nucleotide base comprise the same or different cleavable moieties. In some embodiments, the chain terminating moiety (e.g., the chain terminating moiety located at the sugar 2′ and/or sugar 3′ position) and the detectable reporter moiety linked to the base are chemically cleavable or removable with the same chemical agent. In some embodiments, the chain terminating moiety (e.g., the chain terminating moiety located at the sugar 2′ and/or sugar 3′ position) and the detectable reporter moiety linked to the base are chemically cleavable or removable with different chemical agents.

### Multivalent Molecules

The present disclosure provides methods for sequencing nucleic acid template molecules, where any of the sequencing methods described herein employ at least one multivalent molecule. In some embodiments, the multivalent molecule comprises a plurality of nucleotide arms attached to a core. The multivalent molecule may have any configuration, including a starburst, helter skelter, or bottle brush configuration (see e.g., FIG. 19). In some embodiments, the multivalent molecule comprises: (1) a core; and (2) a plurality of nucleotide arms, each of which comprises (i) a core attachment moiety, (ii) a spacer comprising a PEG moiety, (iii) a linker, and (iv) a nucleotide unit, wherein the core is attached to each nucleotide arm in the plurality of nucleotide arms, wherein the spacer is attached to the linker, and wherein the linker is attached to the nucleotide unit. In some embodiments, the nucleotide unit comprises a base, sugar and at least one phosphate group, and the linker is attached to the nucleotide unit through the base. In some embodiments, the linker comprises an aliphatic chain, wherein the aliphatic chain comprises 2-6 subunits. In some embodiments, the linker comprises an ethylene glycol chain, wherein the ethylene glycol chain comprises 2-6 subunits. In some embodiments, the linker further comprises an aromatic moiety. An illustrative nucleotide arm is shown in FIG. 23. Illustrative multivalent molecules are shown in FIGS. 19-22. An illustrative spacer is shown in FIG. 24 (top) and illustrative linkers are shown in FIG. 24 (bottom panel) and FIG. 25.

Illustrative nucleotides attached to a linker are shown in FIGS. 26A-26C. An illustrative biotinylated nucleotide arm is shown in FIG. 27.

In some embodiments, a multivalent molecule comprises a core attached to multiple nucleotide arms, wherein all of the multiple nucleotide arms comprise the same type of nucleotide unit which is selected from a group consisting of dATP, dGTP, dCTP, dTTP and dUTP.

In some embodiments, a multivalent molecule comprises a core attached to multiple nucleotide arms, wherein individual nucleotide arms comprise nucleotide units. As used herein the term “nucleotide unit” refers to a moiety that includes an aromatic base, preferably a base comprising cytidine, thymidine, adenosine, guanosine, inosine, uridine, or methylated, nitrosylated, acetylated, or otherwise modified variants thereof; a five carbon sugar (e.g., ribose or deoxyribose), and optionally, one or more phosphate, sulfate, or ester groups (e.g., 1-10 phosphate groups). In some embodiments, individual multivalent molecules in the plurality of multivalent molecules comprise the same type of multivalent molecule, which comprises the same type of nucleotide unit selected from a group consisting of dATP, dGTP, dCTP, dTTP and dUTP. In some embodiments, the plurality of multivalent molecules comprises a mixture of two or more types of multivalent molecules, wherein individual multivalent molecules in the mixture comprise nucleotide units selected from a group consisting of dATP, dGTP, dCTP, dTTP and/or dUTP.

In some embodiments, the nucleotide unit comprises a chain of one, two or three phosphorus atoms, wherein the chain is typically attached to the 5′ carbon of the sugar moiety via an ester or phosphoramide linkage. In some embodiments, at least one nucleotide unit present in at least one of the multivalent molecules is a nucleotide analog having a phosphorus chain in which the phosphorus atoms are linked together with intervening O, S, NH, methylene or ethylene. In some embodiments, the phosphorus atoms in the phosphorus chain comprise substituted side groups such as O, S or BH₃. In some embodiments, the phosphorus chain comprise phosphate groups substituted with analogs such as phosphoramidate, phosphorothioate, phosphordithioate, and/or O-methylphosphoroamidite groups.

In some embodiments, a multivalent molecule comprises a core attached to multiple nucleotide arms, wherein individual nucleotide arms comprise a nucleotide unit, which is a nucleotide analog having a chain terminating moiety (e.g., blocking moiety) at the sugar 2′ position. In some embodiments, a multivalent molecule comprises a core attached to multiple nucleotide arms, wherein individual nucleotide arms comprises a nucleotide unit, which is a nucleotide analog having a chain terminating moiety (e.g., blocking moiety) at the sugar 3′ position. In some embodiments, a multivalent molecule comprises a core attached to multiple nucleotide arms, wherein individual nucleotide arms comprises a nucleotide unit, which is a nucleotide analog having a chain terminating moiety (e.g., blocking moiety) at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar position, wherein the sugar comprises a ribose or deoxyribose sugar moiety. In some embodiments, the chain terminating moiety is removable or cleavable from the 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable or removable from the nucleotide unit, for example by reacting the chain terminating moiety with a chemical agent, by a pH change, by light or by heat. In some embodiments, the chain terminating moieties comprising alkyl, alkenyl, alkynyl or allyl are cleavable with tetrakis(triphenylphosphine)palladium(0) (Pd(PPh₃)₄), with piperidine, or with 2,3-Dichloro-5,6-dicyano-1,4-benzo-quinone (DDQ). In some embodiments, the chain terminating moieties comprising aryl or benzyl are cleavable with H2 Pd/C. In some embodiments, the chain terminating moieties comprising amine, amide, keto, isocyanate, phosphate, thio, or disulfide are cleavable with phosphine or with a thiol group such as beta-mercaptoethanol or dithiothritol (DTT). In some embodiments, the chain terminating moieties comprising carbonate are cleavable with potassium carbonate (K₂CO₃) in MeOH, with triethylamine in pyridine, or with Zn in acetic acid (AcOH). In some embodiments, the chain terminating moieties comprising urea or silyl are cleavable with tetrabutylammonium fluoride, with pyridine-HF, with ammonium fluoride, or with triethylamine trihydrofluoride.

In some embodiments, the nucleotide unit comprises a chain terminating moiety (e.g., blocking moiety) at the sugar 2′ position. In some embodiments, the nucleotide unit comprises a chain terminating moiety (e.g., blocking moiety) at the sugar 3′ position. In some embodiments, the nucleotide unit comprises a chain terminating moiety (e.g., blocking moiety) at the sugar 2′ and 3′ positions. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties comprising azide, azido or azidomethyl groups are cleavable or removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the nucleotide unit comprises a chain terminating moiety which is selected from a group consisting of 3′-deoxy nucleotides, 2′,3′-dideoxynucleotides, or a nucleotide that comprises one of the following 3′ modifications: 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein individual nucleotide arms comprise a spacer, a linker and a nucleotide unit, and wherein the core, linker and/or nucleotide unit is labeled with detectable reporter moiety. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, at least one nucleotide arm of a multivalent molecule comprises a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, the core of a multivalent molecule comprises an avidin-like or streptavidin-like moiety and the core attachment moiety comprises biotin. In some embodiments, the core comprises an streptavidin-type or avidin-type moiety, for example, an avidin protein, or a derivative, analog and other non-native form of avidin that can bind to at least one biotin moiety. Other forms of avidin moieties include native and recombinant avidin and streptavidin, as well as derivatized molecules, e.g. non-glycosylated avidin and truncated streptavidins. For example, an avidin moiety may be a de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially available products ExtrAvidin®, CaptAvidin™, NeutrAvidin and Neutralite Avidin.

In some embodiments of the methods for sequencing nucleic acid molecules described herein, the methods comprise forming a binding complex, where the binding complex comprises (i) a polymerase, a nucleic acid template molecule duplexed with a primer, and a nucleotide, or (ii) a polymerase, a nucleic acid template molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.8, 0.9 or 1 second. In some embodiments, the binding complex has a persistence time of greater than about 0.1-0.25 seconds, about 0.25-0.5 seconds, about 0.5-0.75 seconds, about 0.75-1 second, about 1-2 seconds, about 2-3 seconds, about 3-4 second, or about 4-5 seconds. In some embodiments, the method is carried out at a temperature of at or above 15 °C, at or above 20 °C, at or above 25 °C, at or above 35 °C, at or above 37 °C, at or above 42 °C, at or above 55 °C, at or above 60 °C, or at or above 72 °C, or at or above 80 °C. In some embodiments, the method is carried out at a temperature of about 15 °C to about 20 °C, about 20 °C to about 25 °C, about 25 °C to about 35 °C, about 35 °C to about 37 °C, about 37 °C to about 42 °C, about 42 °C to about 55 °C, about 55 °C to about 60 °C, about 60 °C to about 72 °C, or about 72 °C to about 80 °C. The binding complex (e.g., ternary complex) may remain stable until subjected to a condition that causes dissociation of interactions between any of the polymerase, nucleic acid template molecule, primer and/or the nucleotide unit or the nucleotide. For example, a dissociating condition can be brought about by contacting the binding complex with a detergent, EDTA and/or water. In some embodiments, the binding complex is deposited on, attached to, or hybridized to, a surface and the contrast to noise ratio in the detecting step is greater than 20. In some embodiments, the contacting is performed under conditions that stabilize the binding complex when the nucleotide or nucleotide unit is complementary to a next base of the nucleic acid template molecule, and destabilize the binding complex when the nucleotide or nucleotide unit is not complementary to the next base of the nucleic acid template molecule.

### Sample Indexes for Improved Base Calling

Generally, it is desirable to prepare nucleic acid libraries (i.e., libraries of template molecules) that will be distributed onto a support (e.g., coated flowcell), where the library molecules are converted into template molecules that are immobilized at a high density to the support for massively parallel sequencing. For template molecules that are immobilized at high densities at random locations on the support, a major challenge is resolving high density fluorescent images for accurate base calling during sequencing runs. This challenge is exacerbated with the nucleotide diversity of the library is low.

The nucleotide diversity of a population of immobilized template molecules refers to the relative proportion of nucleotides A, G, C and T that are present in each sequencing cycle. An optimal high diversity library will generally comprise sequence-of-interest (insert) regions having approximately equal proportions of all four nucleotides represented in each cycle of a sequencing run. A low diversity library will generally comprise sequence-of-interest (insert) regions having a high proportion of certain nucleotides and low proportion of other nucleotides. To overcome the problem of low diversity libraries, a small amount of a high diversity library prepared from PhiX bacteriophage is typically mixed with the library-of-interest (e.g., a PhiX spike-in library) and sequenced together on the same flowcell. While the PhiX library spike-in library provides nucleotide diversity, it also occupies space on the flowcell, thereby replacing the target libraries carrying the sequence-of-interest and reduces the amount of sequencing data obtainable from the target libraries (i.e., reduces sequencing throughput). Another method to overcome the problem of low diversity libraries is to prepare target library molecules having at least one sample index sequence that is designed to be color-balanced through selection of nucleotides that make up for the nucleotides that are under-represented in the low diversity library. However it may be desirable to design a large number of sample index sets, for example a set of single index sample sequences or paired index sample sequences for 16-plex, 24-plex, 96-plex or larger levels. It is challenging to design sample index sequences, as a single or paired sample indexes, for large sample index sets where all of the sample index sequences different and yet are color-balanced.

An alternative method to overcome the challenges of sequencing low diversity library molecules (e.g., at high density on the support) is to prepare libraries having at least one sample index sequence comprising a short random sequence having 2, 3, 4, 5, 6, 7, 8, 9, 10 or more nucleotides (e.g., NNN) linked directly to a universal sample index sequence, where the short random sequence provides nucleotide diversity and color balance. For example, the right and/or left index sequences present in the template molecules described herein can comprise a short random sequence (e.g., NNN) and a universal sample index sequence. The short random sequence (e.g., NNN) can be located 5′ of the universal sample index sequence. In a population of sample-indexed library molecules, the short random sequence of the sample index provides high nucleotide diversity which comprises approximately equal proportions of all four nucleotides (e.g., A, G, C, T and/or U) that will be represented in each cycle of a sequencing run. The high nucleotide diversity of the short random sequence also provide color balance during each cycle of the sequencing run. The advantage of designing sample indexes to comprise a short random sequence (e.g., NNN) is that, in a low-plexy population of library molecules (e.g., 2-plex or 4-plex), the universal sample index sequences that identify the two or four different samples need not exhibit nucleotide diversity. Additionally, the nucleotide diversity of the short random sequence (e.g., NNN) can obviate the need to include a PhiX spike-in library, or permits use of a reduced amount of PhiX spike-in library to be distributed onto the flowcell and sequenced.

The target library molecule can comprise a single sample index sequence which includes a short random sequence and a universal sample index sequence. In some embodiments, the sequencing data from only the single sample index sequence can be used for polony mapping and template registration because the short random sequence (e.g., NNN) provides sufficient nucleotide diversity and color balance. The sequencing data from the universal sample index sequence can be used to distinguish sequences of interest obtained from different sample sources in a multiplex assay.

The target library molecule can further comprise a second sample index sequence (e.g., dual sample index) comprising a second universal sample index sequence. In some embodiments, the sequencing data from the first and second sample index sequences (e.g., left and right sample indexes) can be used for polony mapping and/or template registration because the short random sequences provides sufficient nucleotide diversity and color balance. The sequencing data from the first universal sample index sequence and the second universal sample index sequence can be used as dual sample indexes to distinguish sequences of interest obtained from different sample sources in a multiplex assay. In some embodiments, the second sample index sequence may or may not comprise a second short random sequence (e.g., NNN).

The order of sequencing the sequence-of-interest region and the sample index region(s) can be varied to improve the challenges of sequencing low diversity library molecules. For example, the sample index region can be sequenced first before sequencing the sequence-of-interest region, and the sample index sequence can be associated with the sequence-of-interest region. As a further example, sample index region can be sequenced first, including sequencing the short random sequence (e.g., NNN), and optionally sequencing at least a portion of the universal sample index), and then sequencing the sequence-of-interest region. In a population of sample indexed library molecules, the short random sequence (e.g., NNN) provides nucleotide diversity which may not be provided the sequence-of-interest regions of the library molecules. The sequence of the sample index provides improved nucleotide diversity and color balance for polony mapping and template registration.

Additionally, when sequencing the sample index region first, the length of the sequenced sample index region is generally relatively short (e.g., less than 30 nucleotides in length), so that de-hybridization of the product of the sequenced sample index region is more complete. Gentler de-hybridization conditions can be used to remove most or all of the product of the sequenced sample index region which reduces the level of residual signals from any sequencing products remaining hybridized to the template molecules. By contrast, the sequence-of-interest region is typically much longer than the sample index region (e.g., more than 100 nucleotides in length). When the sequence-of-interest region is sequenced before the sample index region, the product of the sequenced sequence-of-interest region will be subjected to harsher de-hybridization conditions to remove any products remaining hybridized to the template molecules which may damage the template molecules.

The read-capping technology described herein can be used to incorporate a read-capping nucleotide analog at the terminal 3′ end of any sequencing read product when sequencing a first region of a template molecule is completed, thereby generating a first capped read sequencing read product which is not removed from the template molecule prior to sequencing a second region of the same template molecule. The read-capping technology obviates the need for de-hybridizing sequencing read products and permits sequencing any region of the template molecule in any order. For example, the longer insert region of a template molecule can be sequenced and subjected to read-capping prior to sequencing the shorter index region. Alternatively, at least one of the shorter sample index regions can be sequenced, and optionally subjected to read-capping, prior to sequencing the longer insert region, where sequencing information from the sample index region is used for polony mapping and template registration as described herein. In some embodiments, a short random sequence (e.g., NNN) can be linked to a universal sample index sequence, where the short random sequence provides a high diversity nucleotide sequence and color balance which is used for polony mapping and template registration.

The present disclosure provides a population of nucleic acid template molecules each comprising at least one sample index sequence that can be used to distinguish sequences-of-interest obtained from different sample sources in a multiplex assay, where the at least one sample index sequence comprises a short random sequence (e.g., NNN) linked to a universal sample index sequence. In some embodiments, the left sample index comprises a short random sequence (e.g., NNN) linked to a universal left sample index sequence and/or the right sample index comprises a short random sequence (e.g., NNN) linked to a right universal sample index sequence. The at least one sample index sequence can include sequence diversity for improved base calling. The at least one sample index sequence can be used to improve base calling accuracy.

In some embodiments, the short random sequence (e.g., NNN) is positioned upstream of the universal sample index sequence so that during a sequencing run the random sequence portion is sequenced before the universal sample index sequence. In some embodiments, the short random sequence is positioned downstream of the universal sample index sequence so that during a sequencing run the random portion is sequenced after the universal sample index sequence.

In some embodiments, in the random sequence each base “N” at a given position is independently selected from A, G, C, T or U. In some embodiments, the random sequence lacks consecutive repeat sequences having 2 or 3 of the same nucleobase, for example, the sequence does not comprise AA, TT, CC, GG, UU, AAA, TTT, CCC, GGG or UUU. In some embodiments, in a population of template molecules, the universal sample index sequences comprise a short random sequence having a high diversity sequence which comprises approximately equal proportions of all four nucleotides (e.g., A, G, C, T and/or U) that will be represented in each cycle of a sequencing run.

In some embodiments, the short random sequence (e.g., NNN) comprises 3-20 nucleotides, or 3-10 nucleotides, or 3-8 nucleotides, or 3-6 nucleotides, or 3-5 nucleotides, or 3-4 nucleotides.

In some embodiments, the short random sequence (e.g., NNN) comprises or consists of AGC, AGT, GAC, GAT, CAT, CAG, TAG, TAC. The skilled artisan will recognize that many more random sequences can be prepared (e.g., 64 possible combinations if the random sequence is 3 nucleotides long) where each base “N” at a given position in the random sequence is independently selected from A, G, C, T or U.

In some embodiments, the universal sample index sequence comprises 5-20 nucleotides, or 7-18 nucleotides, or 9-16 nucleotides.

In some embodiments, individual right sample index sequences (e.g., first sample index sequences, see e.g. FIGS. 1 and 2) in a population of right sample index sequences comprise a universal sample index sequence and a short random sequence (e.g., NNN). In some embodiments, the short random sequences in the population of right sample index sequences have an overall base composition of about 25% or about 20-30% of all four nucleotide bases (e.g., A, G, C and T/U) to provide nucleotide diversity at each sequencing cycle during sequencing the short random sequence (e.g., NNN).

In some embodiments, in the population of right sample index sequences the proportion of adenine (A) in the short random sequence is about 20-30% or about 15-35% or about 10-40%. In some embodiments, in the population of right sample index sequences the proportion of guanine (G) at any given position in the short random sequence is about 20-30% or about 15-35% or about 10-40%. In some embodiments, in the population of right sample index sequences the proportion of cytosine (C) at any given position in the short random sequence is about 20-30% or about 15-35% or about 10-40%. In some embodiments, in the population of right sample index sequences the proportion of thymine (T) or uracil (U) at any given position in the short random sequence is about 20-30% or about 15-35% or about 10-40%.

In some embodiments, in the population of right sample index sequences the proportion of adenine (A) and thymine (T), or the proportion of adenine (A) and uracil (U) in the short random sequence is about 10-65%. In some embodiments, in the population of right sample index sequences the proportion of guanine (G) and cytosine (C) at any given position in the short random sequence is about 10-65%.

In some embodiments, in the population of right sample index sequences the sequence diversity of the short random sequences ensures that no sequencing cycle is presented with fewer than four different nucleotide bases during sequencing at least the short random sequence (e.g., NNN).

Illustrative sample index sequence that comprise a short random sequence linked directly to a universal sample index sequence include but are not limited to: NNNGTAGGAGCC; NNNCCGCTGCTA; NNNAACAACAAG; NNNGGTGGTCTA; NNNTTGGCCAAC; NNNCAGGAGTGC; and NNNATCACACTA, wherein N can be any nucleotide. The skilled artisan will recognize that the universal sample index can be any length and may comprise any sequence that can be used to distinguish sequences of interest obtained from different sample sources in a multiplex assay. In a population of a given sample index, for example NNNGTAGGAGCC, the population contains a mixture of individual sample index molecules each carrying the same universal sample index sequence (e.g., GTAGGAGCC) and a different short random sequence (e.g., NNN) where up to 64 different short random sequences NNN may be present in the population of the given sample index.

In some embodiments, at least one template molecule comprises a right sample index comprising a short random sequence (e.g., NNN) directly linked to a right universal sample index sequence. In some embodiments, the at least one template molecule comprises a left sample index comprising a left universal sample index sequence that differs from the right universal sample index sequence.

In some embodiments, the random sequence (e.g., NNN) provides a balanced ratio of the nucleobases adenine, cytosine, guanine, thymine and/or uracil. In some embodiments, in a population of sample-indexed template molecules, the random sequence (e.g., NNN) together with at least a portion of the universal sample index sequence provides a balanced ratio of the nucleobases adenine, cytosine, guanine, thymine and/or uracil represented in each cycle of a sequencing run.

In some embodiments, a sequencing reaction comprises the use of polymerases and nucleotides (e.g., nucleotide analogs) that are labeled with a different fluorophores that correspond to the nucleobase identities. In some embodiments, sequencing the random sequence (e.g., NNN) using labeled nucleotides provides a balanced ratio of fluorescent colors that correspond to the nucleobases adenine, cytosine, guanine, thymine and/or uracil in each cycle of a sequencing run. In some embodiments, sequencing the random sequence (e.g., NNN) and at least a portion of the universal sample index sequence using labeled nucleotides provides a balanced ratio of fluorescent colors that correspond to the nucleobase identities adenine, cytosine, guanine, thymine and/or uracil. The labeled nucleotides emit fluorescent signals during the sequencing reactions. In some embodiments, the sequencing reaction is conducted on a sequencing apparatus having a detector that captures fluorescent images from sequencing reactions on the immobilized template molecules. The sequencing apparatus can be configured to relay the fluorescent imaging data captured by the detector to a computer system that is programmed to determine the location (e.g., mapping) of the immobilized template molecules on the flowcell. Immobilized template molecule locations can be based on the fluorescent signals, or any other methods known in the art. The computer system can generate a map of the locations of the immobilized template molecules based on the fluorescent imaging data of only the random sequence (e.g., NNN), or based on the random sequence (e.g., NNN) and at least a portion the universal sample index sequence. Thus the few numbers of sequencing cycles used to sequence the random sequence (e.g., NNN) and optionally a portion of the universal sample index sequence can be used to generate a map of the location of the immobilized template molecules. The computer system can be configured to extract the fluorescent color and intensity of only the random sequence (e.g., NNN), or the random sequence (e.g., NNN) and at least a portion of the universal sample index sequence. The computer system can be further configured to use the location of a given immobilized template molecule and the fluorescent color and intensity associated with the given template molecule (which were established while sequencing the random sequence) for base calling while sequencing the insert region (e.g., sequence-of-interest). The computer system can be configured to detect phasing and pre-phasing while sequencing the random sequence (e.g., NNN) and the universal sample index sequence, and the insert region. In some embodiments, the balanced ratio of fluorescent colors provided by the random sequence (e.g., NNN) at each sequencing cycle can improve the quality of the data which is processed from the fluorescent images captured by the detector, and can in turn improve the capability by the computer system to determine the location of the immobilized template molecules on the flowcell, and the color and intensity, all of which can improve base calling accuracy and quality scores of the sequenced insert region.

In some embodiments, a sequencing reaction comprises the use of polymerases and multivalent molecules, wherein individual multivalent molecules comprise a core attached to multiple nucleotide arms and individual nucleotide arms are attached to a nucleotide unit, that is labeled with a different fluorophore that corresponds to the nucleobase (e.g., adenine, guanine, cytosine, thymine or uracil) of the nucleotide unit. In some embodiments, the core of individual multivalent molecules is attached to a fluorophore which corresponds to the nucleotide unit (e.g., adenine, guanine, cytosine, thymine or uracil) that are attached to the nucleotide arms in a multivalent molecule. In such embodiments, the nucleotide units attached to an individual multivalent molecule all have the same identity (e.g., all adenine, or all guanine and the like). In some embodiments, at least one of the nucleotide arms of the multivalent molecule comprises a linker and/or nucleotide base that is attached to a fluorophore, wherein the fluorophore which is attached to a given linker or nucleotide base that corresponds to the nucleotide base (e.g., adenine, guanine, cytosine, thymine or uracil) of the nucleotide arm. In some embodiments, sequencing the random sequence (e.g., NNN) using labeled multivalent molecules provides a balanced ratio of fluorescent colors that correspond to the nucleobases adenine, cytosine, guanine, thymine and/or uracil in each cycle of a sequencing run. In some embodiments, sequencing the random sequence (e.g., NNN) and at least a portion of the universal sample index sequence using labeled multivalent molecules provides a balanced ratio of fluorescent colors that correspond to the nucleobases adenine, cytosine, guanine, thymine and/or uracil. The labeled multivalent molecules may emit fluorescent signals during the sequencing reactions. In some embodiments, the sequencing reaction is conducted on a sequencing apparatus having a detector that captures fluorescent images from sequencing reactions on the immobilized template molecules. The sequencing apparatus can be configured to relay the fluorescent imaging data captured by the detector to a computer system that is programmed to determine the location (e.g., mapping) of the immobilized template molecules (polonies) on the flowcell. The computer system can generate a map of the locations of the immobilized template molecules based on the fluorescent imaging data of only the random sequence (e.g., NNN), or based on the random sequence (e.g., NNN) and at least a portion of the universal sample index sequence. Thus the few numbers of sequencing cycles used to sequence the random sequence (e.g., NNN) and optionally a portion of the universal sample index sequence can be used to generate a map of the location of the immobilized template molecules. The computer system can be configured to extract the fluorescent color and intensity of only the random sequence (e.g., NNN) or the random sequence (e.g., NNN) and the universal sample index sequence. The computer system can be configured to use the location of a given immobilized template molecule and the fluorescent color and intensity associated with the given template molecule (which were established while sequencing the random sequence) for base calling while sequencing the insert region. The computer system can be configured to detect phasing and pre-phasing while sequencing the random sequence (e.g., NNN) and the universal sample index sequence, and the insert region. In some embodiments, the balanced ratio of fluorescent colors provided by the random sequence (e.g., NNN) at each sequencing cycle can improve the quality of the data which is processed from the fluorescent images captured by the detector, and can in turn improve the capability by the computer system to determine the location of the immobilized template molecules on the flowcell, and the color and intensity, all of which can improve base calling accuracy and quality scores of the sequenced insert region.

### A First Embodiment: Order of Sequencing Sample Index Sequences

In some embodiments, the order of sequencing is as follows: (1) sequencing the right sample index, wherein the right index comprises a first random sequence (e.g., NNN) and a right universal sample index sequence; (2) sequencing the left sample index; and (3) sequencing the insert region. In some embodiments, the left sample index comprises a left universal sample index sequence. In some embodiments, the left sample index comprises a second random sequence (e.g., NNN) and a left universal sample index sequence. In some embodiments, sequencing the right sample index region, including the first random sequence (e.g., NNN) and right universal sample index sequence, may provide enough nucleotide diversity so that sequencing the left sample index can be omitted.

In some embodiments, individual nucleic acid template molecules comprise: (i) a universal binding sequence for a second surface primer, (ii) a left sample index sequence having a universal left sample index sequence, (iii) a universal binding sequence for a forward sequencing primer, (iv) a sequence of interest, (v) a universal binding sequence for a reverse sequencing primer, (vi) a right sample index sequence having a short random sequence (e.g., NNN) linked directly to a right universal sample index sequence, and (vii) a universal binding sequence for a first surface primer (e.g., see FIG. 1). In some embodiments, the methods comprises step (a): contacting the nucleic acid template molecules with a first plurality of soluble sequencing primers that hybridize to the universal binding sequence for a reverse sequencing primer, and sequencing the right sample index sequence comprising sequencing the short random sequence (e.g., NNN) and the right universal sample index sequence, thereby generating a first plurality of sample index extension products that are hybridized to the nucleic acid template molecules, wherein the first plurality of sample index extension products are complementary to the right sample index sequence having a short random sequence (e.g., NNN) linked directly to a right universal sample index sequence.

In some embodiments, the methods for sequencing comprise step (b): retaining the first plurality of sample index extension products and retaining the nucleic acid template molecules.

In some embodiments, the methods for sequencing comprise step (c): contacting the nucleic acid template molecules retained at step (b) with a second plurality of soluble sequencing primers that hybridize to the universal binding sequence for the second surface primer and sequencing the left sample index sequence, thereby generating a second plurality of sample index extension products that are hybridized to the nucleic acid template molecules, wherein the second plurality of sample index extension products are complementary to the left sample index sequence having a left universal sample index sequence.

In some embodiments, the methods for sequencing comprise step (d): retaining the second plurality of sample index extension products and retaining the nucleic acid template molecules.

In some embodiments, the methods for sequencing comprise step (e): contacting the nucleic acid template molecules retained at step (d) with a third plurality of soluble sequencing primers that hybridize to the universal binding sequence for the forward sequencing primer and sequencing the insert region, thereby generating a plurality of insert extension products that are hybridized to the nucleic acid template molecules, wherein the plurality of insert extension products are complementary to the sequence of interest.

In some embodiments, the methods for sequencing further comprise step (f1): assigning the sequence of the insert region to the right sample index sequence having a short random sequence (e.g., NNN) linked directly to a right universal sample index sequence, thereby identifying the insert region as being obtained from a first source.

In some embodiments, the methods for sequencing comprise step (f2): assigning the sequence of the insert region to the right sample index sequence having a short random sequence (e.g., NNN) linked directly to a right universal sample index sequence, and to the left sample index sequence, thereby identifying the insert region as being obtained from a first source.

In some embodiments, the steps (a), (c) and (e) comprise conducting any of the sequencing methods described herein that employ sequencing polymerases and detectably labeled nucleotide analogs. In some embodiments, the sequencing of steps (a), (c) and (e) comprise conducting any of the two-stage sequencing methods described herein that employ sequencing polymerases, detectably labeled multivalent molecules, and nucleotide analogs. In some embodiments, the sequencing of steps (a), (c) and (e) comprise conducting any of the sequencing-by-binding methods described herein.

In some embodiments, the nucleic acid template molecules are immobilized. In some embodiments, the density of the plurality of nucleic acid template molecules immobilized to the support is about 10²-10¹⁵ per mm². In some embodiments, the plurality of nucleic acid template molecules are immobilized at random locations on the support. In some embodiments, the plurality of nucleic acid template molecules are immobilized on the support in a predetermined pattern.

### A Second Embodiment: Order of Sequencing Sample Index Sequences

In some embodiments, the order of sequencing is as follows: (1) sequencing the right sample index, wherein the right sample index comprises a random sequence (e.g., NNN) and a universal sample index sequence; (2) sequencing the insert region; and (3) sequencing the left sample index. In some embodiments, the left sample index comprises a left universal sample index sequence. In some embodiments, the left sample index comprises a second random sequence (e.g., NNN) and a left universal sample index sequence. In some embodiments, sequencing the right sample index region, including the first random sequence (e.g., NNN) and right universal sample index sequence, may provide enough nucleotide diversity so that sequencing the left sample index can be omitted.

In some embodiments, individual nucleic acid template molecules comprise: (i) a universal binding sequence for a second surface primer, (ii) a left sample index sequence having a left universal sample index sequence (iii) a universal binding sequence for a forward sequencing primer, (iv) a sequence of interest, (v) a universal binding sequence for a reverse sequencing primer, (vi) a right sample index sequence having a short random sequence (e.g., NNN) linked directly to a right universal sample index sequence, and (vii) a universal binding sequence for a first surface primer (e.g., see FIG. 1). In some embodiments, the method comprises step (a): contacting the nucleic acid template molecules with a first plurality of soluble sequencing primers that hybridize to the universal binding sequence for a reverse sequencing primer, and sequencing the right sample index sequence including sequencing the short random sequence (e.g., NNN) and the right universal sample index sequence, thereby generating a first plurality of sample index extension products that are hybridized to the nucleic acid template molecules, wherein the first plurality of sample index extension products are complementary to the right sample index sequence having a short random sequence (e.g., NNN) linked directly to a right universal sample index sequence.

In some embodiments, the methods comprise step (b): retaining the first plurality of sample index extension products and retaining the template molecules.

In some embodiments, the methods comprise step (c): contacting the template molecules retained at step (b) with a second plurality of soluble sequencing primers that hybridize to the universal binding sequence for a forward sequencing primer and sequencing the insert region thereby generating a plurality of insert extension products that are hybridized to the nucleic acid template molecules, wherein the plurality of insert extension products are complementary to the sequence of interest.

In some embodiments, the methods comprise step (d): retaining the plurality of insert extension products and retaining the nucleic acid template molecules.

In some embodiments, the methods comprise step (e): contacting the nucleic acid template molecules retained at step (d) with a third plurality of soluble sequencing primers that hybridize to the universal binding sequence for a second surface primer and sequencing the left sample index sequence thereby generating a second plurality of sample index extension products that are hybridized to the nucleic acid template molecules, wherein the second plurality of sample index extension products are complementary to the left sample index sequence having a left universal sample index sequence.

In some embodiments, the methods comprise step (f1): assigning the sequence of the insert region to the right sample index sequence having a short random sequence (e.g., NNN) linked directly to a right universal sample index sequence, thereby identifying the insert region as being obtained from a first source.

In some embodiments, the methods comprise step (f2): assigning the sequence of the insert region to the right sample index sequence having a short random sequence (e.g., NNN) linked directly to a right universal sample index sequence, and to the left sample index sequence, thereby identifying the insert region as being obtained from a first source.

In some embodiments, steps (a), (c) and (e) comprise conducting any of the sequencing methods described herein that employ sequencing polymerases and detectably labeled nucleotide analogs. In some embodiments, steps (a), (c) and (e) comprise conducting any of the two-stage sequencing methods described herein that employ sequencing polymerases, detectably labeled multivalent molecules, and nucleotide analogs. In some embodiments, steps (a), (c) and (e) comprise conducting any of the sequencing-by-binding methods described herein.

In some embodiments, the nucleic acid template molecules are immobilized. In some embodiments, the density of the plurality of nucleic acid template molecules immobilized to the support is about 10²-10¹⁵ per mm². In some embodiments, the plurality of nucleic acid template molecules are immobilized at random locations on the support. In some embodiments, the plurality of nucleic acid template molecules are immobilized on the support in a predetermined pattern.

### A Third Embodiment: Order of Sequencing Sample Index Sequences

In some embodiments, the order of sequencing is as follows: (1) sequencing the insert region; (2) sequencing the right sample index, wherein the right sample index comprises a random sequence (e.g., NNN) and a universal sample index sequence; and (3) sequencing the left sample index. In some embodiments, the left sample index comprises a left universal sample index sequence. In some embodiments, the left sample index comprises a second random sequence (e.g., NNN) and a left universal sample index sequence. In some embodiments, sequencing the right sample index region, including the first random sequence (e.g., NNN) and right universal sample index sequence, may provide enough nucleotide diversity so that sequencing the left sample index can be omitted.

In some embodiments, individual template molecules comprise: (i) a universal binding sequence for a second surface primer, (ii) a left sample index sequence having a left universal sample index sequence (iii) a universal binding sequence for a forward sequencing primer, (iv) a sequence of interest, (v) a universal binding sequence for a reverse sequencing primer, (vi) a right sample index sequence having a short random sequence (e.g., NNN) linked directly to a right universal sample index sequence, and (vii) a universal binding sequence for a first surface primer (e.g., see FIG. 1). In some embodiments, the method comprises step (a): contacting the nucleic acid template molecules with a first plurality of soluble sequencing primers that hybridize to the universal binding sequence for a forward sequencing primer, and sequencing the insert region, thereby generating a plurality of insert extension products that are hybridized to the nucleic acid template molecules, wherein the plurality of insert extension products are complementary to the sequence of interest.

In some embodiments, the methods comprise step (b): retaining the plurality of insert extension products and retaining the nucleic acid template molecules.

In some embodiments, the methods comprise step (c): contacting the nucleic acid template molecules retained at step (b) with a second plurality of soluble sequencing primers that hybridize to the universal binding sequence for a reverse sequencing primer and sequencing the right sample index sequence including sequencing the short random sequence (e.g., NNN) and the right universal sample index sequence thereby generating a first plurality of sample index extension products that are hybridized to the nucleic acid template molecules, wherein the first plurality of sample index extension products are complementary to the right sample index sequence having a short random sequence (e.g., NNN) linked directly to a right universal sample index sequence.

In some embodiments, the methods comprise step (d): retaining the first plurality of sample index extension products and retaining the nucleic acid template molecules.

In some embodiments, the methods comprise step (e): contacting the nucleic acid template molecules retained at step (d) with a third plurality of soluble sequencing primers that hybridize to the universal binding sequence for a second surface primer and sequencing the left sample index sequence, thereby generating a second plurality of sample index extension products that are hybridized to the template molecules, wherein the second plurality of sample index extension products are complementary to the left sample index sequence having a left universal sample index sequence.

In some embodiments, the methods comprise step (f1): assigning the sequence of the insert region to the right sample index sequence having a short random sequence (e.g., NNN) linked directly to a right universal sample index sequence, thereby identifying the insert region as being obtained from a first source.

In some embodiments, the methods comprise step (f2): assigning the sequence of the insert region to the right sample index sequence having a short random sequence (e.g., NNN) linked directly to a right universal sample index sequence, and to the left sample index sequence, thereby identifying the insert region as being obtained from a first source.

In some embodiments, steps (a), (c) and (e) comprise conducting any of the sequencing methods described herein that employ sequencing polymerases and detectably labeled nucleotide analogs. In some embodiments, steps (a), (c) and (e) comprise conducting any of the two-stage sequencing methods described herein that employ sequencing polymerases, detectably labeled multivalent molecules, and nucleotide analogs. In some embodiments, (a), (c) and (e) comprise conducting any of the sequencing-by-binding methods described herein.

In some embodiments, the nucleic acid template molecules are immobilized. In some embodiments, the density of the plurality of nucleic acid template molecules immobilized to the support is about 10²-10¹⁵ per mm². In some embodiments, the plurality of nucleic acid template molecules are immobilized at random locations on the support. In some embodiments, the plurality of nucleic acid template molecules are immobilized on the support in a predetermined pattern.

### A Fourth Embodiment: Order of Sequencing

In some embodiments, the order of sequencing is as follows: (1) sequencing the first 3-5 bases of the insert region; (2) sequencing the right sample index where the right sample index comprises an optional random sequence (e.g., NNN) and a universal sample index sequence; and (3) sequencing the left sample index. In some embodiments, the left sample index comprises a left universal sample index sequence. In some embodiments, the left sample index comprises a second random sequence (e.g., NNN) and a left universal sample index sequence. In some embodiments, sequencing the first 3-5 bases of the insert region may provide enough sequence diversity so that the right sample index and the left sample index do not comprise a short random sequence (e.g., NNN). In some embodiments, sequencing the right sample index region, including the first random sequence (e.g., NNN) and right universal sample index sequence, may provide enough nucleotide diversity so that sequencing the left sample index can be omitted.

In some embodiments, individual nucleic acid template molecules comprise: (i) a universal binding sequence for a second surface primer, (ii) a left sample index sequence having a left universal sample index sequence (iii) a universal binding sequence for a forward sequencing primer, (iv) a sequence of interest, (v) a universal binding sequence for a reverse sequencing primer, (vi) a right sample index sequence having a short random sequence (e.g., NNN) linked directly to a right universal sample index sequence, and (vii) a universal binding sequence for a first surface primer (e.g., see FIG. 1). In some embodiments, the method comprises step (a): contacting the nucleic acid template molecules with a first plurality of soluble sequencing primers that hybridize to the universal binding sequence for a forward sequencing primer, and sequencing the first 3-5 bases of the insert region, thereby generating a plurality of insert extension products that are hybridized to the nucleic acid template molecules, wherein the each of the insert extension products is complementary to the sequence of interest.

The sequence of the first 3-5 bases of the insert region may provide sufficient sequence diversity and color balance for polony mapping and template registration.

In some embodiments, the methods comprise step (b): retaining the plurality of insert extension products and retaining the nucleic acid template molecules.

In some embodiments, the methods comprise step (c): contacting the template molecules retained at step (b) with a second plurality of soluble sequencing primers that hybridize to the universal binding sequence for a reverse sequencing primer, and sequencing the right sample index sequence including sequencing the short random sequence (e.g., NNN) if present and the right universal sample index sequence, thereby generating a first plurality of sample index extension products that are hybridized to the nucleic acid template molecules, wherein the first plurality of sample index extension products are complementary to the right sample index sequence.

In some embodiments, the methods comprise step (d): retaining the first plurality of sample index extension products and retaining the nucleic acid template molecules.

In some embodiments, the methods comprise step (e): contacting the nucleic acid template molecules retained at step (d) with a third plurality of soluble sequencing primers that hybridize to the universal binding sequence for a second surface primer and sequencing the left sample index sequence, thereby generating a second plurality of sample index extension products that are hybridized to the nucleic acid template molecules, wherein the second plurality of sample index extension products are complementary to the left sample index sequence having a left universal sample index sequence.

In some embodiments, the methods comprise step (f): retaining the second plurality of sample index extension products and retaining the nucleic acid template molecules.

In some embodiments, the methods comprise step (g): contacting the nucleic acid template molecules retained at step (f) with a fourth plurality of soluble sequencing primers that hybridize to the universal binding sequence for a forward sequencing primer and sequencing the full length of the insert region, thereby generating a plurality of full length insert extension products that are hybridized to the nucleic acid template molecules, wherein the plurality of full length insert extension products are complementary to the sequence of interest.

In some embodiments, the methods comprise step (h1): assigning the full length sequence of the insert region to the right sample index sequence, thereby identifying the insert region as being obtained from a first source.

In some embodiments, the methods comprise step (h2): assigning the full length sequence of the insert region to the right sample index sequence, and to the left sample index sequence, thereby identifying the insert region as being obtained from a first source.

In some embodiments, steps (a), (c), (e) and (g) comprise conducting any of the sequencing methods described herein that employ sequencing polymerases and detectably labeled nucleotide analogs. In some embodiments, steps (a), (c), (e) and (g) comprise conducting any of the two-stage sequencing methods described herein that employ sequencing polymerases, detectably labeled multivalent molecules, and nucleotide analogs. In some embodiments, steps (a), (c), (e) and (g) comprise conducting any of the sequencing-by-binding methods described herein.

In some embodiments, the nucleic acid template molecules are immobilized. In some embodiments, the density of the plurality of nucleic acid template molecules immobilized to the support is about 10²-10¹⁵ per mm². In some embodiments, the plurality of nucleic acid template molecules are immobilized at random locations on the support. In some embodiments, the plurality of template molecules are immobilized on the support in a predetermined pattern.

### A Fifth Embodiment: Order of Sequencing

In some embodiments, the order of sequencing is as follows: (1) sequencing the first 3-5 bases of the insert region of the nucleic acid template molecule (e.g., sequencing in a forward direction) which is immobilized; (2) sequencing the right sample index where the right sample index comprises a first random sequence (e.g., NNN) and a right universal sample index sequence; (3) sequencing the left sample index; (4) conducting a pairwise turn reaction so that the immobilized template molecule is replaced with an immobilized strand that is complementary to the nucleic acid template molecule; and (5) sequencing the full-length of the insert region of the immobilized complementary strand (e.g., sequencing in the reverse direction). In some embodiments, the sequences of the first 3-5 bases of the insert region of a population of template molecules may provide enough sequence diversity for improved base-calling accuracy. In some embodiments, the left sample index comprises a left universal sample index sequence. In some embodiments, the left sample index comprises a second random sequence (e.g., NNN) and a left universal sample index sequence. In some embodiments, sequencing the right sample index region, including the first random sequence (e.g., NNN) and right universal sample index sequence, may provide enough nucleotide diversity so that sequencing the left sample index can be omitted.

In some embodiments, individual nucleic acid template molecules are covalently linked to an immobilized capture primer that lacks uracil bases. In some embodiments, individual template molecules comprise randomly-distributed uracil bases, and individual template molecules comprise: (i) a universal binding sequence for a second surface primer, (ii) a left sample index sequence having a left universal sample index sequence (iii) a universal binding sequence for a forward sequencing primer, (iv) a sequence of interest, (v) a universal binding sequence for a reverse sequencing primer, (vi) a right sample index sequence having a short random sequence (e.g., NNN) linked directly to a right universal sample index sequence, and (vii) a universal binding sequence for a first surface primer (e.g., see FIG. 1). In some embodiments, the method comprises step (a): contacting the nucleic acid template molecules with a first plurality of soluble sequencing primers (e.g., forward sequencing primers) that hybridize to the universal binding sequence for a forward sequencing primer, and sequencing the first 3-5 bases of the insert region, thereby generating a plurality of insert extension products that are hybridized to the nucleic acid template molecules, wherein the plurality of insert extension products are complementary to the sequence of interest.

In some embodiments, the methods comprise step (b): retaining the plurality of insert extension products and retaining the nucleic acid template molecules.

In some embodiments, the methods comprise step (c): contacting the template molecules retained at step (b) with a second plurality of soluble sequencing primers that hybridize to the universal binding sequence for a reverse sequencing primer, and sequencing the right sample index sequence including sequencing the short random sequence (e.g., NNN) and the right universal sample index sequence, thereby generating a first plurality of sample index extension products that are hybridized to the nucleic acid template molecules, wherein the first plurality of sample index extension products are complementary to the right sample index sequence having a short random sequence (e.g., NNN) linked directly to a right universal sample index sequence.

In some embodiments, the methods comprise step (d): retaining the first plurality of sample index extension products and retaining the nucleic acid template molecules.

In some embodiments, the methods comprise step (e): contacting the nucleic acid template molecules retained at step (d) with a third plurality of soluble sequencing primers that hybridize to the universal binding sequence for a second surface primer, and sequencing the left sample index sequence, thereby generating a second plurality of sample index extension products that are hybridized to the nucleic acid template molecules, wherein the second plurality of sample index extension products are complementary to the left sample index sequence having a left universal sample index sequence.

In some embodiments, the methods comprise step (f): replacing the second plurality of sample index extension products that are hybridized to the nucleic acid template molecules by conducting a primer extension reaction using strand-displacing polymerases and a plurality of nucleotides to generate an extension product that is hybridized to the nucleic acid template molecules including the immobilized capture primer.

In some embodiments, the methods comprise step (g): removing the nucleic acid template molecules by generating abasic sites in the nucleic acid template molecules at the uracil sites and generating gaps at the abasic sites, thereby generating gap-containing nucleic acid template molecules while retaining the extension products that were generated in step (f), wherein individual extension products are retained by being hybridized to an immobilized capture primer. In some embodiments, pairwise turn is achieved by conducting steps (g) and (h) (described below).

In some embodiments, comprise step (h): contacting the extension products retained at step (g) with a fourth plurality of soluble sequencing primers (e.g., reverse sequencing primers) that hybridize to universal binding sequence for a reverse sequencing primer, and sequencing the insert region (e.g., sequencing at least a portion or the full length of the insert region).

In some embodiments, the methods comprise step (il): assigning the sequence of the insert region to the right sample index sequence having a short random sequence (e.g., NNN) linked directly to a right universal sample index sequence, thereby identifying the insert region as being obtained from a first source.

In some embodiments, the methods comprise step (i2): assigning the sequence of the insert region to the right sample index sequence having a short random sequence (e.g., NNN) linked directly to a right universal sample index sequence, and to the left sample index sequence, thereby identifying the insert region as being obtained from a first source.

In some embodiments, steps (a), (c), (e) and (h) comprise conducting any of the sequencing methods described herein that employ sequencing polymerases and detectably labeled nucleotide analogs. In some embodiments, steps (a), (c), (e) and (h) comprise conducting any of the two-stage sequencing methods described herein that employ sequencing polymerases, detectably labeled multivalent molecules, and nucleotide analogs. In some embodiments, steps (a), (c), (e) and (h) comprise conducting any of the sequencing-by-binding methods described herein.

In some embodiments, the plurality of nucleic acid template molecules is immobilized. In some embodiments, the density of the plurality of nucleic acid template molecules immobilized to the support is about 10²-10¹⁵ per mm². In some embodiments, the plurality of nucleic acid template molecules are immobilized at random locations on the support. In some embodiments, the plurality of nucleic acid template molecules are immobilized on the support in a predetermined pattern.

### Supports With Low Non-specific Binding Coatings

The present disclosure provides compositions and methods comprising a support having a plurality of surface primers immobilized thereon, for preparing any of the immobilized template molecules described herein. In some embodiments, the support is passivated with a low non-specific binding coating (see e.g., FIG. 18). In some embodiments, the surface coatings described herein exhibit very low non-specific binding to reagents typically used for nucleic acid capture, amplification and sequencing workflows, such as dyes, nucleotides, enzymes, and nucleic acid primers. In some embodiments, the surface coatings exhibit low background fluorescence signals or high contrast-to-noise (CNR) ratios compared to conventional surface coatings.

In general, the supports may comprise a substrate (or support structure), one or more layers of a covalently or non-covalently attached low-binding, chemical modification layers, e.g., silane layers, polymer films, and one or more covalently or non-covalently attached primer sequences that may be used for tethering single-stranded target nucleic acid(s) to the support surface. In some embodiments, the formulation of the surface, e.g., the chemical composition of one or more layers, the coupling chemistry used to cross-link the one or more layers to the support surface and/or to each other, and the total number of layers, may be varied such that non-specific binding of proteins, nucleic acid molecules, and other hybridization and amplification reaction components to the support surface is minimized or reduced relative to a comparable monolayer. Often, the formulation of the surface may be varied such that non-specific hybridization on the support surface is minimized or reduced relative to a comparable monolayer. The formulation of the surface may be varied such that non-specific amplification on the support surface is minimized or reduced relative to a comparable monolayer. The formulation of the surface may be varied such that specific amplification rates and/or yields on the support surface are maximized. Amplification levels suitable for detection are achieved in no more than 2, 3, 4, 5, 6, 7, 8, 9, 10, 15, 20, 25, 30, or more than 30 amplification cycles in some cases disclosed herein.

The substrate or support structure that comprises the one or more chemically-modified layers, e.g., layers of a low non-specific binding polymer, may be independent or integrated into another structure or assembly. For example, in some embodiments, the substrate or support structure may comprise one or more surfaces within an integrated or assembled microfluidic flowcell. The substrate or support structure may comprise one or more surfaces within a microplate format, e.g., the bottom surface of the wells in a microplate. As noted above, in some preferred embodiments, the substrate or support structure comprises the interior surface (such as the lumen surface) of a capillary. In alternate preferred embodiments the substrate or support structure comprises the interior surface (such as the lumen surface) of a capillary etched into a planar chip.

The attachment chemistry used to graft a first chemically-modified layer to a surface will generally be dependent on both the material from which the surface is fabricated and the chemical nature of the layer. In some embodiments, the first layer may be covalently attached to the surface. In some embodiments, the first layer may be non-covalently attached, e.g., adsorbed to the surface through non-covalent interactions such as electrostatic interactions, hydrogen bonding, or van der Waals interactions between the surface and the molecular components of the first layer. In either case, the substrate surface may be treated prior to attachment or deposition of the first layer. Any of a variety of surface preparation techniques known to those of skill in the art may be used to clean or treat the surface. For example, glass or silicon surfaces may be acid-washed using a Piranha solution (a mixture of sulfuric acid (H₂SO₄) and hydrogen peroxide (H₂O₂)), base treatment in KOH and NaOH, and/or cleaned using an oxygen plasma treatment method.

Silane chemistries constitute one non-limiting approach for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding surfaces include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the surface, where the choice of components used may be varied to alter one or more properties of the surface, e.g., the surface density of functional groups and/or tethered oligonucleotide primers, the hydrophilicity/hydrophobicity of the surface, or the three three-dimensional nature (i.e., “thickness”) of the surface. Examples of preferred polymers that may be used to create one or more layers of low non-specific binding material in any of the disclosed surfaces include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the substrate. Alternately, the surface coating may be patterned, such that the chemical modification layers are confined to one or more discrete regions of the substrate. For example, the surface may be patterned using photolithographic techniques to create an ordered array or random pattern of chemically-modified regions on the surface. Alternately or in combination, the substrate surface may be patterned using, e.g., contact printing and/or ink-jet printing techniques. In some embodiments, an ordered array or random pattern of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In order to achieve low nonspecific binding surfaces, hydrophilic polymers may be nonspecifically adsorbed or covalently grafted to the surface. Typically, passivation is performed utilizing poly(ethylene glycol) (PEG, also known as polyethylene oxide (PEO) or polyoxyethylene) or other hydrophilic polymers with different molecular weights and end groups that are linked to a surface using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface. In some embodiments, two or more layers may be covalently coupled to each other or internally cross-linked to improve the stability of the resulting surface. In some embodiments, oligonucleotide primers with different base sequences and base modifications (or other biomolecules, e.g., enzymes or antibodies) may be tethered to the resulting surface layer at various surface densities. In some embodiments, for example, both surface functional group density and oligonucleotide concentration may be varied to target a certain primer density range. Additionally, primer density can be controlled by diluting oligonucleotide with other molecules that carry the same functional group. For example, amine-labeled oligonucleotide can be diluted with amine-labeled polyethylene glycol in a reaction with an NHS-ester coated surface to reduce the final primer density. Primers with different lengths of linker between the hybridization region and the surface attachment functional group can also be applied to control surface density.

Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In order to scale primer surface density and add additional dimensionality to hydrophilic or amphoteric surfaces, surfaces comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the surface significantly.

Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially.

Examples of suitable polymers include, but are not limited to, streptavidin, poly acrylamide, polyester, dextran, poly-lysine, and copolymers of poly-lysine and PEG. In some embodiments, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some embodiments, high primer density materials may be constructed in solution and subsequently layered onto the surface in multiple steps.

As noted, the low non-specific binding coatings of the present disclosure may exhibit reduced non-specific binding of proteins, nucleic acids, and other components of the hybridization and/or amplification formulation used for solid-phase nucleic acid amplification.

The degree of non-specific binding exhibited by a given support surface may be assessed either qualitatively or quantitatively. For example, in some embodiments, exposure of the surface to fluorescent dyes (e.g., cyanine dyes such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations provided that care has been taken to ensure that the fluorescence imaging is performed under a condition where fluorescence signal is linearly related (or related in a predictable manner) to the number of fluorophores on the support surface (e.g., under a condition where signal saturation and/or self-quenching of the fluorophore is not an issue) and suitable calibration standards are used. In some embodiments, other techniques known to those of skill in the art, for example, radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

As noted, in some embodiments, the degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some embodiments, the label may comprise a fluorescent label. In some embodiments, the label may comprise a radioisotope. In some embodiments, the label may comprise any other detectable label known to one of skill in the art. In some embodiments, the degree of non-specific binding exhibited by a given support surface formulation may thus be assessed in terms of the number of non-specifically bound protein molecules (or other molecules) per unit area. In some embodiments, the low-binding supports of the present disclosure may exhibit non-specific protein binding (or non-specific binding of other specified molecules, (e.g., cyanine dyes such as Cy3, or Cy5, etc., fluoresceins, coumarins, rhodamines, etc. or other dyes disclosed herein)) of less than 0.001 molecule per μm2, less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². Those of skill in the art will recognize that a given support surface of the present disclosure may exhibit non-specific binding falling anywhere within this range, for example, of less than 86 molecules per μm². In some embodiments, some modified surfaces disclosed herein exhibit nonspecific protein binding of less than 0.5 molecule/μm² following contact with a 1 μM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm². In independent nonspecific binding assays, 1 μM labeled Cy3 SA (ThermoFisher), 1 μM Cy5 SA dye (ThermoFisher), 10 μM Aminoallyl-dUTP-ATTO-647N (Jena Biosciences), 10 μM Aminoallyl-dUTP-ATTO-Rho 11 (Jena Biosciences), 10 μM Aminoallyl-dUTP-ATTO-Rho 11 (Jena Biosciences), 10 μM 7-Propargylamino-7-deaza-dGTP-Cy5 (Jena Biosciences, and 10 μM 7-Propargylamino-7-deaza-dGTP-Cy3 (Jena Biosciences) were incubated on the low binding substrates at 37 °C for 15 minutes in a 384 well plate format. Each well was rinsed 2-3× with 50 ul deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (Olympus Corp., Center Valley, PA) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, New York), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm².

In some embodiments, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value. In some embodiments, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value.

The low-background surfaces described herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces disclosed herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles. In this method, a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein is no more than 50 degrees, no more than 40 degrees, no more than 30 degrees, no more than 25 degrees, no more than 20 degrees, no more than 18 degrees, no more than 16 degrees, no more than 14 degrees, no more than 12 degrees, no more than 10 degrees, no more than 8 degrees, no more than 6 degrees, no more than 4 degrees, no more than 2 degrees, or no more than 1 degree. In some embodiments, the contact angle is no more than 40 degrees. Those of skill in the art will recognize that a given hydrophilic, low-binding support surface of the present disclosure may exhibit a water contact angle having a value of anywhere within this range.

In some embodiments, the hydrophilic surfaces disclosed herein facilitate reduced wash times for bioassays, often due to reduced nonspecific binding of biomolecules to the low-binding surfaces. In some embodiments, adequate wash steps may be performed in less than 60, 50, 40, 30, 20, 15, 10, or less than 10 seconds per step. For example, in some embodiments adequate wash steps may be performed in less than 30 seconds per step.

In some embodiments, the low-binding surfaces of the present disclosure exhibit significant improvement in stability or durability to prolonged exposure to solvents and elevated temperatures, or to repeated cycles of solvent exposure or changes in temperature. For example, in some embodiments, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces may exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface.

In some embodiments, fluorescence images of the disclosed low background surfaces when used in nucleic acid hybridization or amplification applications to create clusters of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer (e.g., surface capture primers) may be attached or tethered to the support surface. In some embodiments, two types of primer (e.g., surface capture primers and surface pinning primers) may be attached or tethered to the support surface. In some embodiments, the one or more types of adapters or primers may comprise spacer sequences, adapter sequences for hybridization to adapter-ligated target library nucleic acid sequences, forward amplification primers, reverse amplification primers, sequencing primers, and/or molecular barcoding sequences, or any combination thereof. In some embodiments, 1 primer or adapter sequence may be tethered to at least one layer of the surface. In some embodiments, at least 2, 3, 4, 5, 6, 7, 8, 9, 10, or more than 10 different primer or adapter sequences may be tethered to at least one layer of the surface.

In some embodiments, the tethered adapter and/or primer sequences may range in length from about 10 nucleotides to about 100 nucleotides. In some embodiments, the tethered adapter and/or primer sequences may be at least 10, at least 20, at least 30, at least 40, at least 50, at least 60, at least 70, at least 80, at least 90, or at least 100 nucleotides in length. In some embodiments, the tethered adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some embodiments the length of the tethered adapter and/or primer sequences may range from about 20 nucleotides to about 80 nucleotides. in some embodiments the length of the tethered adapter and/or primer sequences may range from about 10 nucleotides to about 20 nucleotides. In some embodiments the length of the tethered adapter and/or primer sequences may range from about 20 nucleotides to about 30 nucleotides. In some embodiments the length of the tethered adapter and/or primer sequences may range from about 30 nucleotides to about 40 nucleotides. In some embodiments the length of the tethered adapter and/or primer sequences may range from about 40 nucleotides to about 50 nucleotides. In some embodiments the length of the tethered adapter and/or primer sequences may range from about 50 nucleotides to about 60 nucleotides. In some embodiments the length of the tethered adapter and/or primer sequences may range from about 60 nucleotides to about 70 nucleotides. In some embodiments the length of the tethered adapter and/or primer sequences may range from about 70 nucleotides to about 80 nucleotides. In some embodiments the length of the tethered adapter and/or primer sequences may range from about 80 nucleotides to about 90 nucleotides. In some embodiments the length of the tethered adapter and/or primer sequences may range from about 90 nucleotides to about 100 nucleotides Those of skill in the art will recognize that the length of the tethered adapter and/or primer sequences may have any value within this range, e.g., about 24 nucleotides.

In some embodiments, the resultant surface density of primers on the low binding support surfaces of the present disclosure may range from about 100 primer molecules per μm² to about 100,000 primer molecules per μm². In some embodiments, the resultant surface density of primers on the low binding support surfaces of the present disclosure may range from about 100,000 primer molecules per μm² to about 10¹⁵ primer molecules per μm². In some embodiments, the surface density of primers may be at least 1,000, at least 10,000, at least 100,000, or at least 10¹⁵ primer molecules per μm². In some embodiments, the surface density of primers may be at most 10,000, at most 100,000, at most 1,000,000, or at most 10¹⁵ primer molecules per μm². Any of the lower and upper values described in this paragraph may be combined to form a range included within the present disclosure, for example, in some embodiments the surface density of primers may range from about 10,000 molecules per μm² to about 10¹⁵ molecules per μm². Those of skill in the art will recognize that the surface density of primer molecules may have any value within this range, e.g., about 455,000 molecules per μm². In some embodiments, the surface density of nucleic acid template molecules initially hybridized to adapter or primer sequences on the support surface may be less than or equal to that indicated for the surface density of tethered primers. In some embodiments, the surface density of nucleic acid template molecules hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000 per μm², while also comprising at least a second region having a substantially different local density.

In some embodiments, the low non-specific binding coating comprising one or more layers of a multi-layered surface coating may comprise a branched polymer. In some embodiments, the low non-specific binding coating comprising one or more layers of a multi-layered surface coating may comprise a linear polymer. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo(ethylene glycol) methyl ether methacrylate) (branched POEGMA), branched polyglutamic acid (branched PGA), branched poly-lysine, branched poly-glucoside, and dextran.

In some embodiments, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branches.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some embodiments, e.g., wherein at least one layer of a multi-layered surface comprises a branched polymer, the number of covalent bonds between a branched polymer molecule of the layer being deposited and molecules of the previous layer may range from about one covalent linkage per molecule to about 32 covalent linkages per molecule. In some embodiments, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some embodiments, the number of layers is at least 1, at least 2, at least 3, at least 4, at least 5, at least 6, at least 7, at least 8, at least 9, or at least 10. In some embodiments, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some embodiments the number of layers may range from about 2 to about 4. In some embodiments the number of layers may range from about 4 to about 6. In some embodiments the number of layers may range from about 6 to about 8. In some embodiments the number of layers may range from about 8 to about 10. In some embodiments, all of the layers may comprise the same material. In some embodiments, each layer may comprise a different material. In some embodiments, the plurality of layers may comprise a plurality of materials. In some embodiments at least one layer may comprise a branched polymer. In some embodiment, all of the layers may comprise a branched polymer.

One or more layers of low non-specific binding material may in some cases be deposited on and/or conjugated to the substrate surface using a polar protic solvent, a polar or polar aprotic solvent, a nonpolar solvent, or any combination thereof. In some embodiments the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino)propanesulfonic acid (MOPS), etc.), or any combination thereof. In some embodiments, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total solvent mixture, with the balance made up of water or an aqueous buffer solution. In some embodiments, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total solvent mixture, with the balance made up of an organic solvent. The pH of the solvent mixture used may be less than 6, about 6, 6.5, 7, 7.5, 8, 8.5, 9, or greater than pH 9.

Fluorescence imaging may be performed using any of a variety of fluorophores, fluorescence imaging techniques, and fluorescence imaging instruments known to those of skill in the art. Examples of suitable fluorescence dyes that may be used (e.g., by conjugation to nucleotides, oligonucleotides, or proteins) include, but are not limited to, fluorescein, rhodamine, coumarin, cyanine, and derivatives thereof, including the cyanine derivatives Cyanine dye-3 (Cy3), Cyanine dye-5 (Cy5), and Cyanine dye-7 (Cy7). Examples of fluorescence imaging techniques that may be used include, but are not limited to, fluorescence microscopy imaging, fluorescence confocal imaging, two-photon fluorescence, and the like. Examples of fluorescence imaging instruments that may be used include, but are not limited to, fluorescence microscopes equipped with an image sensor or camera, confocal fluorescence microscopes, two-photon fluorescence microscopes, or custom instruments that comprise a suitable selection of light sources, lenses, mirrors, prisms, dichroic reflectors, apertures, and image sensors or cameras. A non-limiting example of a fluorescence microscope equipped for acquiring images of the disclosed low-binding support surfaces and clonally-amplified colonies (polonies) of template nucleic acid sequences hybridized thereon is the Olympus IX83 inverted fluorescence microscope equipped with 20×, 0.75 NA, a 532 nm light source, a bandpass and dichroic mirror filter set optimized for 532 nm long-pass excitation and Cy3 fluorescence emission filter, a Semrock 532 nm dichroic reflector, and a camera (Andor sCMOS, Zyla 4.2), where the excitation light intensity is adjusted to avoid signal saturation. Often, the support surface may be immersed in a buffer (e.g., 25 mM ACES, pH 7.4 buffer) while the image is acquired.

In some instances, the performance of nucleic acid hybridization and/or amplification reactions using the disclosed reaction formulations and low non-specific binding supports may be assessed using fluorescence imaging techniques, where the contrast-to-noise ratio (CNR) of the images provides a key metric in assessing amplification specificity and non-specific binding on the support. CNR is commonly defined as: CNR=(Signal−Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times must be minimized), as shown in the example below. The surfaces of the instant disclosure are also provided in WO 2020/102594, which is hereby incorporated by reference in its entirety for examples of surfaces that may be used in the supports disclosed here.

In most ensemble-based sequencing approaches, the background term is typically measured as the signal associated with ‘interstitial’ regions. In addition to “interstitial” background (Bᵢₙₜₑᵣ), “intrastitial” background (Bᵢₙₜᵣₐ) exists within the region occupied by an amplified DNA colony. The combination of these two background signals may dictate the achievable CNR, and thus directly impacts the optical instrument requirements, architecture costs, reagent costs, run-times, cost/genome, and ultimately the accuracy and data quality for cyclic array-based sequencing applications. The Bᵢₙₜₑᵣ background signal may arise from a variety of sources; a few examples include auto-fluorescence from consumable flowcells, non-specific adsorption of detection molecules that yield spurious fluorescence signals that may obscure the signal from the ROI, the presence of non-specific DNA amplification products (e.g., those arising from primer dimers). In typical next generation sequencing (NGS) applications, this background signal in the current field-of-view (FOV) is averaged over time and subtracted. The signal arising from individual DNA colonies (i.e., (S)-Bᵢₙₜₑᵣ in the FOV) yields a discernable feature that can be classified. In some instances, the intrastitial background (Bᵢₙₜᵣₐ) can contribute a confounding fluorescence signal that is not specific to the target of interest, but is present in the same ROI thus making it far more difficult to average and subtract.

The implementation of nucleic acid amplification on the low-binding substrates of the present disclosure may decrease the Binter background signal by reducing non-specific binding, may lead to improvements in specific nucleic acid amplification, and may lead to a decrease in non-specific amplification that can impact the background signal arising from both the interstitial and intrastitial regions. In some instances, the disclosed low-binding support surfaces, optionally used in combination with the disclosed hybridization buffer formulations, may lead to improvements in CNR by a factor of 2, 5, 10, 100, or 1000-fold over those achieved using conventional supports and hybridization, amplification, and/or sequencing protocols. Although described here in the context of using fluorescence imaging as the read-out or detection mode, the same principles apply to the use of the disclosed low non-specific binding supports and nucleic acid hybridization and amplification formulations for other detection modes as well, including both optical and non-optical detection modes.

The disclosed low-binding supports, optionally used in combination with the disclosed hybridization and/or amplification protocols, may yield solid-phase reactions that exhibit: (i) negligible non-specific binding of protein and other reaction components (thus minimizing substrate background), (ii) negligible non-specific nucleic acid amplification product, and (iii) provide tunable nucleic acid amplification reactions.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

In some embodiments, a fluorescence image of the surface exhibits a contrast-to-noise ratio (CNR) of at least 20 when a sample nucleic acid molecule or complementary sequences thereof are labeled with a Cyanine dye-3 (Cy3) fluorophore, and when the fluorescence image is acquired using an inverted fluorescence microscope (e.g., Olympus IX83) with a 20×0.75 NA objective, a 532 nm light source, a bandpass and dichroic mirror filter set optimized for 532 nm excitation and Cy3 fluorescence emission, and a camera (e.g., Andor sCMOS, Zyla 4.2) under non-signal saturating conditions while the surface is immersed in a buffer (e.g., 25 mM ACES, pH 7.4 buffer).

### EXAMPLES

The following examples are meant to be illustrative and can be used to further understand embodiments of the present disclosure and should not be construed as limiting the scope of the present teachings in any way.

### EXAMPLE 1: Generating Capped Sequencing Read Products

Coated flowcells comprising a multi-layered polymer coating and tethered capture primers are used to prepare immobilized concatemer molecules. A library of linear nucleic acid molecules having a sequence-of-interest and universal adaptor sequences are circularized. The circularized library molecules are distributed onto the coated flowcells and hybridized to the capture primers. On-support rolling circle amplification is conducted to generate concatemer molecules immobilized to the coating on the support.

A two-stage sequencing reaction is conducted. The first-stage is conducting by flowing sequencing primers onto the immobilized concatemers in the flowcell to hybridize to their respective sequencing primer binding sites along the concatemer molecules and form template/primer duplexes. Sequencing polymerases and fluorescently labeled multivalent molecules (e.g., a mixture of A, G, C and U) are flowed onto the flowcells such that the polymerases to bind the template/primer duplexes to form complexed polymerases, and the detectably labeled multivalent molecules bind the complexed polymerases to form multivalent-polymerase complexes. The conditions also inhibit incorporation of the nucleotide units of the multivalent molecules by including a non-catalytic divalent cation. The flowcells are washed to remove unbound multivalent molecules. The washed flowcells are imaged to detect the labeled multivalent molecules that remained bound to the complexed polymerases. The first sequencing polymerases and multivalent molecules are removed by washing, while retaining the sequencing primers hybridized to the concatemers (retained duplexes).

The second-stage sequencing reaction is conducted by contacting the retained duplexes with second sequencing polymerases to form complexed polymerases. A mixture of non-labeled nucleotide analogs (e.g., 3′O-methylazido nucleotides) is added to the flowcell in the presence of a buffer that comprises a catalytic cation, and the result is incubated such that the nucleotide analogs bind complementary nucleotides in the complexed polymerases/retained duplexes and promote polymerase-catalyzed incorporation of the nucleotides to generate a nascent extended sequencing primer. The complexed polymerases are washed. The incorporated nucleotide analogs are reacted with a cleaving reagent that removes the 3′ O-methylazido group and generates an extendible 3′OH group. The second sequencing polymerases are removed by washing, while retaining the nascent extended sequencing primers hybridized to the concatemers. Recurring sequencing reactions are conducted by performing multiple cycles of first-stage and second-stage sequencing reactions to generate extended forward sequencing primer strands.

A capping reaction is conducted by contacting the extended forward sequencing primer strands with a plurality of the second sequencing polymerase and a mixture of read-capping nucleotide analogs (e.g., A, G, C or U) comprising a base-linked PEG terminating moiety, and a buffer having a catalytic cation. The read-capping nucleotide analogs comprise either —OH or —H at the 3′ sugar position. The capping reaction is incubated, such that polymerase-catalyzed incorporation of the read-capping nucleotides to generate capped sequencing read products occurs. The capped sequencing read products remain hybridized to their respective template molecules. The capped sequencing read products are washed to remove excess read-capping nucleotide analogs.

To test the blocking/capping ability of the incorporated read-capping nucleotide analogs, the capped sequencing read products are contacted with a plurality of the first sequencing polymerases and one type of fluorescently labeled multivalent molecules that are expected to bind to the complexed polymerases. The conditions are suitable for binding the detectably labeled multivalent molecules to the complexed polymerases but inhibit incorporation of the nucleotide units of the multivalent molecules. The flowcells are washed to remove unbound multivalent molecules. The washed flowcells are imaged to detect any labeled multivalent molecules that remained bound to the complexed polymerases, and the intensity is measured.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

What is claimed:

1. A method for sequencing two or more regions of a nucleic acid template molecule, comprising:
a) providing a plurality of nucleic acid template molecules, wherein individual template molecules comprise (i) a first region and a first universal sequencing primer binding site and (ii) a second region and a second universal sequencing primer binding site;
b) providing a plurality of first nucleic acid sequencing primers and a plurality of second nucleic acid sequencing primers;
c) hybridizing individual first nucleic acid sequencing primers to the first universal sequencing primer binding sites on the template molecules;
d) sequencing the first regions of the plurality of nucleic acid template molecules, thereby generating a plurality of first sequencing read products;
e) conducting a plurality of first capping reactions comprising incorporating a read-capping nucleotide analog into the terminal 3′ ends of individual first sequencing read products, thereby generating a plurality of capped first sequencing read products,
wherein the read-capping nucleotide analog comprises (i) a heterocyclic base, (ii) a sugar, and (iii) a polyphosphate chain,
wherein the heterocyclic base is linked to a terminating moiety that blocks binding of a multivalent molecule to the terminal 3′ ends of the capped first sequencing read products, wherein individual multivalent molecules comprise a core attached to multiple nucleotide arms and individual nucleotide arms are attached to a nucleotide unit; and
wherein the terminating moiety blocks polymerase-catalyzed incorporation of a subsequent nucleotide into the capped first sequencing read products,
f) retaining the plurality of capped first sequencing read products which are hybridized to nucleic acid template molecules, wherein the terminating moiety is not cleaved or removed;
g) hybridizing individual second nucleic acid sequencing primers to the second universal sequencing primer binding sites on the individual template molecules; and
h) sequencing the second regions on the plurality of template molecules, thereby generating a plurality of second sequencing read products.

2. The method of claim 1, further comprising:
i) conducting a plurality of second capping reactions comprising incorporating a read-capping nucleotide analog into the terminal 3′ ends of individual second sequencing read products, thereby generating a plurality of capped second sequencing read products,
wherein the read-capping nucleotide analog comprises (i) a heterocyclic base, (ii) a sugar, and (iii) a polyphosphate chain,
wherein the heterocyclic base is linked to a terminating moiety that blocks binding of a multivalent molecule to the terminal 3′ ends of the capped second sequencing read products, wherein individual multivalent molecules comprise a core attached to multiple nucleotide arms and individual nucleotide arms are attached to a nucleotide unit;
wherein the terminating moiety blocks polymerase-catalyzed incorporation of a subsequent nucleotide into the capped second sequencing read products; and
j) retaining the plurality of capped second sequencing read products which are hybridized to nucleic acid template molecules, wherein the terminating moiety is not cleaved or removed.

3. The method of claim 2, wherein individual sequenced template molecules comprise at least one capped first sequencing read product hybridized thereon, and at least one capped second sequencing read product hybridized thereon.

4. The method of any one of claims 1-3, wherein the read-capping nucleotide analog that is incorporated at the terminal 3′ end of individual capped first sequencing read products is not modified to convert the sugar 3′ position into an extendible 3′OH group.

5. The method of claim 2, wherein the read-capping nucleotide analog that is incorporated at the terminal 3′ end of individual capped second sequencing read products is not modified to convert the sugar 3′ position into an extendible 3′OH group.

6. The method of any one of claims 1-5, wherein the heterocyclic base is linked to the terminating moiety by an alkyne linkage, alkene linkage, or alkane linkage.

7. The method of any one of claims 1-6, wherein the terminating moiety comprises a polymer moiety.

8. The method of claim 7, wherein the polymer moiety is selected from the group consisting of a polyether moiety, a polyethylene glycol moiety, polypropylene glycol moiety, polyvinyl acetate moiety, polylactic acid moiety, polyglycolic acid moiety, a polyamide and a polyester moiety.

9. The method of claim 7, wherein the polymer moiety comprises a poly(glycerol) moiety, a poly(oxazoline) moiety, a poly(hydroxypropyl methacrylate) moiety (PHPMA), a poly(2-hydroxyethyl methacrylate) moiety (PHEMA), a poly(N-(2-hydroxypropyl) methacrylamide) moiety (HPMA), a poly(vinylpyrrolidone) moiety (PVP), a poly(N, N-dimethyl acrylamide) moiety (PDMA), or a poly(N-acryloylmorpholine) moiety (PACM).

10. The method of any one of claims 1-9, wherein the terminating moiety comprises a polyethylene glycol (PEG) moiety.

11. The method of claim 10, wherein the PEG moiety has a molecular weight of 1K-20K.

12. The method of any one of claims 1-9, wherein the terminating moiety comprises a propargylamino moiety, an allylamino moiety, a propylamino moiety, an ethylmercapto moiety, a hydroxymethyl moiety, an arylmercapto moiety, a 1-X-1H-1,2,3-triazol-4-yl moiety, a 5-X-1H-1,2,3-triazol-1-methyl moiety, a hydrazone moiety or an O-alkyl oxime moiety, wherein “X” comprises a polymer.

13. The method of any one of claims 1-5, wherein the heterocyclic base comprises a chain terminating moiety at the 3′ sugar group, and wherein the chain terminating moiety comprises an alkyl group, alkenyl group, alkynyl group, allyl group, aryl group, benzyl group, azide group, azido group, O-azidomethyl group, amine group, amide group, keto group, isocyanate group, phosphate group, thio group, disulfide group, carbonate group, urea group, or silyl group.

14. The method of any one of claims 1-13, wherein individual nucleic acid template molecules in the plurality of nucleic acid template molecules are single-stranded or double-stranded nucleic acid molecules.

15. The method of any one of claims 1-14, wherein the plurality of nucleic acid template molecules are immobilized to a support or immobilized to a coating on a support.

16. The method of claim 15, wherein individual nucleic acid template molecules in the plurality of nucleic acid template molecules are covalently joined to an immobilized surface capture primer, or wherein individual template molecules in the plurality are hybridized to an immobilized surface capture primer, wherein the immobilized surface capture primer is immobilized on the support.

17. The method of any one of claims 1-16, wherein at least one of the nucleic acid template molecules in the plurality of nucleic acid template molecules comprises a uridine, or wherein at least one of the nucleic acid template molecules in plurality of nucleic acid template molecules lacks a uridine.

18. The method of any one of claims 1-17, wherein individual nucleic acid template molecules in the plurality of plurality of nucleic acid template molecules comprise clonally amplified nucleic acid molecules.

19. The method of any one of claims 1-18, wherein individual nucleic acid template molecules in the plurality of nucleic acid template molecules comprise at least one copy of a sequence-of-interest and at least one universal adaptor sequence.

20. The method of any one of claims 1-19, wherein individual nucleic acid template molecules in the plurality of nucleic acid template molecules comprise concatemer template molecules having two or more tandem repeat units, wherein individual repeat units comprise a sequence-of-interest and at least one universal adaptor sequence.

21. The method of any one of claims 15-20, wherein the support comprises glass or plastic.

22. The method of any one of claims 15-21, wherein the support is configured on a flowcell, or an interior of a capillary lumen.

23. The method of any one of claims 15-22, wherein the support comprises at least one hydrophilic polymer coating layer and a plurality of surface capture primers immobilized to the at least one hydrophilic polymer coating layer, and wherein the at least one hydrophilic polymer coating layer has a water contact angle of no more than 45 degrees.

24. The method of claim 23, wherein the at least one hydrophilic polymer coating layer comprises polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, or dextran.

25. The method of claim 23 or 24, wherein the at least one hydrophilic polymer coating layer comprises polymer molecules having a molecular weight of at least 1000 Daltons.

26. The method of any one of claims 23-25, wherein the at least one hydrophilic polymer coating layer comprises branched polymer molecules having 4-8 branches.

27. The method of any one of claims 23-26, wherein the support comprises: a) a first coating layer comprising a first monolayer of hydrophilic polymer molecules tethered to the support; b) a second coating layer comprising a second monolayer of hydrophilic polymer molecules tethered to the first monolayer; and c) a third coating layer comprising a third monolayer of hydrophilic polymer molecules tethered to the second monolayer, and wherein the hydrophilic polymer molecules of the first layer, second layer or third layer comprise branched polymer layers.

28. The method of claim 27, wherein the plurality of surface capture primers are immobilized to the hydrophilic polymer molecules of the second monolayer or third monolayer, and the surface capture primers are distributed at a plurality of depths throughout the second layer or the third layer.

29. The method of any one of claims 22-27, wherein one or more of the at least one hydrophilic polymer coating layers comprise a plurality of surface capture primers at a surface density of least 1000/μm².

30. The method of any one of claims 1-29, comprising contacting the plurality of nucleic acid template molecules with (i) the first plurality of sequencing primers, wherein the individual nucleic acid template molecules are immobilized and the plurality of sequencing primers are soluble, (ii) a plurality of sequencing polymerases, and (iii) a plurality of nucleotide reagents, under conditions suitable for hybridizing soluble sequencing primers to individual nucleic acid template molecules to generate a plurality of nucleic acid duplexes on the individual nucleic acid template molecules, and wherein the conditions are suitable for binding nucleic acid duplexes with sequencing polymerases and nucleotide reagents.

31. The method of claim 30, wherein individual nucleotide reagents in the plurality of nucleotide reagents comprise an aromatic base, a five carbon sugar and 1-10 phosphate groups.

32. The method of claim 30 or 31, wherein the plurality of nucleotide reagents comprises one or more types of nucleotide reagent selected from the group consisting of dATP, dGTP, dCTP, dTTP and dUTP.

33. The method of any one of claims 30-32, wherein the plurality of nucleotide reagents comprises a combination of two or more types of nucleotide reagent selected from the group consisting of dATP, dGTP, dCTP, dTTP and dUTP.

34. The method of any one of claims 30-33, wherein at least one nucleotide reagent in the plurality of nucleotide reagents lacks a detectable reporter moiety.

35. The method of any one of claims 30-34, wherein at least one nucleotide reagent in the plurality of nucleotide reagents is labeled with a detectable reporter moiety.

36. The method of any one of claims 30-35, wherein individual nucleotide reagents in the plurality of nucleotide reagents comprise at least one chain terminating nucleotide comprising (i) an aromatic base, (ii) a sugar having a 3′ chain terminating moiety that inhibits polymerase-catalyzed nucleotide incorporation, and (iii) 1-10 phosphate groups.

37. The method of claim 36, wherein the at least one chain terminating nucleotide comprises a removable chain terminating moiety at the 3′ sugar group, and wherein the removable chain terminating moiety comprises an alkyl group, alkenyl group, alkynyl group, allyl group, aryl group, benzyl group, azide group, azido group, O-azidomethyl group, amine group, amide group, keto group, isocyanate group, phosphate group, thio group, disulfide group, carbonate group, urea group, or silyl group.

38. The method of claim 36, wherein the at the at least one chain terminating nucleotide comprises a removable chain terminating moiety at the 3′ sugar group, and wherein the removable chain terminating moiety comprises a 3′-O-amino group, a 3′-O-aminomethyl group, a 3′-O-methylamino group, or derivatives thereof.

39. The method of any one of claims 36-38, wherein the at least one chain terminating moiety is cleavable or removable with a chemical compound to generate an extendible 3′ OH moiety on the sugar group.

40. The method of any one of claims 1-39, wherein sequencing the first region on the plurality of template molecules comprises:
a) contacting the plurality of nucleic acid template molecules with (i) a first plurality of sequencing polymerases and (ii) the plurality of first nucleic acid sequencing primers, wherein the nucleic acid template molecules are immobilized and the plurality of first nucleic acid sequencing primers are soluble, and wherein the contacting is conducted under conditions suitable to form a plurality of complexed polymerases comprising a sequencing polymerase bound to a nucleic acid duplex, wherein the nucleic acid duplex comprises a portion of an individual nucleic acid template molecule hybridized to an individual first nucleic acid sequencing primer;
b) contacting the plurality of complexed sequencing polymerases with a plurality of nucleotides under conditions suitable for binding at least one nucleotide to a complexed sequencing polymerase, wherein the plurality of nucleotides comprises at least one nucleotide analog labeled with a fluorophore and having a removable chain terminating moiety at the sugar 3′ position;
c) incorporating at least one nucleotide into the 3′ ends of the first nucleic acid sequencing primers, thereby generating a plurality of first sequencing read products by extending the first nucleic acid sequencing primers; and
d) detecting the at least one nucleotide and identifying the nucleobase of the at least one nucleotide.

41. The method of any one of claims 30-39, wherein the plurality of nucleotide reagents comprises at least one multivalent molecule.

42. The method of claim 41, wherein the at least one multivalent molecule comprises: (1) a core; and (2) a plurality of nucleotide arms comprising (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, and wherein the linker is attached to the nucleotide unit.

43. The method of claim 42, wherein the nucleotide unit comprises a base, sugar and 1-10 phosphate groups, and the linker is attached to the nucleotide unit through the base.

44. The method of claim 42 or 43, wherein individual nucleotide arms of the at least one multivalent molecule comprise a linker having an aliphatic chain or an oligo ethylene glycol chain, and wherein the linker chain comprises 2-6 subunits.

45. The method of any one of claims 42-44, wherein the core of the at least one multivalent molecule comprises streptavidin and the core attachment moiety comprises biotin.

46. The method of any one of claims 42-45, wherein the plurality of nucleotide arms comprise the same type of a nucleotide unit, and wherein the type of nucleotide unit is selected from the group consisting of dATP, dGTP, dCTP, dTTP and dUTP.

47. The method of any one of claims 42-46, wherein the plurality of nucleotide reagents comprises a plurality of multivalent molecules, wherein individual multivalent molecules in the plurality of multivalent molecules comprise the same type of nucleotide unit.

48. The method of claim 47, wherein the type of nucleotide unit is selected from the group consisting of dATP, dGTP, dCTP, dTTP and dUTP.

49. The method of any one of claims 42-48, wherein the plurality of nucleotide reagents comprises a plurality of multivalent molecules comprising a mixture of two or more types of multivalent molecules, each type of multivalent molecules comprising one or more nucleotide unit types selected from the group consisting of dATP, dGTP, dCTP, dTTP and dUTP.

50. The method of any one of claims 42-49, wherein the plurality of nucleotide reagents comprises at least one fluorophore-labeled multivalent molecule.

51. The method of any one of claims 42-50, wherein sequencing the first regions comprises conducting a two-step sequencing method.

52. The method of claim 51, wherein a first step comprises:
a) contacting the plurality of nucleic acid template molecules with (i) a first plurality of sequencing polymerases and (ii) the plurality of first sequencing primers, wherein the nucleic acid template molecules are immobilized and the plurality of first sequencing primers are soluble, and wherein the contacting is conducted under conditions suitable to form a plurality of first complexed polymerases, individual complexed polymerases comprising a sequencing polymerase bound to a nucleic acid duplex, wherein the nucleic acid duplex comprises a portion of the nucleic acid template molecule hybridized to a first soluble sequencing primer;
b) contacting the plurality of complexed sequencing polymerases with a plurality of nucleotide reagents comprising at least one multivalent molecule, wherein the at least one multivalent molecule is a detachably labeled multivalent molecule, wherein complementary nucleotide units of the multivalent molecules bind to at least two of the plurality of first complexed polymerases, thereby forming a plurality of multivalent-complexed polymerases, and wherein incorporation of the complementary nucleotide units into the first sequencing primers is inhibited;
c) detecting the plurality of multivalent-complexed polymerases; and
d) identifying the nucleobase of the complementary nucleotide units that are bound to the plurality of first complexed polymerases in the plurality of multivalent-complexed polymerases, thereby determining the sequence of the nucleic acid template.

53. The method of claim 52, wherein the complementary nucleotide units are complementary to a nucleotide of the nucleic acid template molecule that is immediately 5′ of the nucleic acid duplex.

54. The method of any one of claims 42-51, wherein sequencing the first region on the plurality of template molecules comprises forming an avidity complex, wherein the method comprises:
(i) binding a first sequencing primer, a first sequencing polymerase, and a first detectably labeled multivalent molecule to a first region of a concatemer template molecule, thereby forming a first binding complex, wherein a first nucleotide unit of the first detectably labeled multivalent molecule binds to the first sequencing polymerase;
(ii) binding a second sequencing primer, a second sequencing polymerase, and the first detectably labeled multivalent molecule to a second region of the concatemer template molecule, thereby forming a second binding complex, wherein a second nucleotide unit of the first multivalent molecule binds to the second sequencing polymerase, wherein the first and second binding complexes form an avidity complex, wherein the concatemer template molecule comprises two or more tandem repeat units, wherein each tandem repeat unit comprises a sequence of interest and a universal primer binding site that binds a first and a second universal sequencing primer, and wherein the contacting is conducted under conditions suitable to inhibit polymerase-catalyzed incorporation of the first and second nucleotide units into the first and second binding complexes, respectively;
(iii) detecting the first and second binding complexes on the same concatemer template molecule, and
(iv) identifying the first nucleotide unit in the first binding complex thereby determining the sequence of the first region of the concatemer template molecule, and identifying the second nucleotide unit in the second binding complex thereby determining the sequence of the second region of the concatemer template molecule.

55. The method of claim 51 or 52, wherein the second step comprises:
e) dissociating the plurality of multivalent-complexed polymerases and removing the plurality of first sequencing polymerases and multivalent molecules, and retaining the nucleic acid duplexes;
f) contacting the nucleic acid duplexes of step (e) with a plurality of second sequencing polymerases, wherein the contacting is conducted under conditions suitable for binding the plurality of second sequencing polymerases to the nucleic acid duplexes, thereby forming a plurality of second complexed polymerases;
g) contacting the plurality of second complexed polymerases with a plurality of nucleotides comprising at least one nucleotide analog having a removable chain terminating moiety, wherein the contacting is conducted under conditions suitable for binding complementary nucleotides present in the plurality of nucleotides to at least two complexed polymerases of the plurality of second complexed polymerases of step (f), thereby forming a plurality of nucleotide-complexed polymerases, and wherein the conditions are suitable for promoting incorporation of the bound complementary nucleotides into the first sequencing primers of the nucleotide-complexed polymerase, thereby generating the plurality of first sequencing read products.

56. The method of claim 55, wherein the removable chain terminating moiety is at the sugar 3′ position.

57. The method of claim 55 or 56, wherein the complementary nucleotides are complementary to a nucleotide of the nucleic acid template molecule that is immediately 5′ of the nucleic acid duplex.
